(19) United States
(12) Patent Application Publication
(10) Pub. No.: US 2015/0265695 A1
(43) Pub. Date: Sep. 24, 2015

(54) VACCINE COMPOSITIONS FOR PREVENTION AGAINST DENGUE VIRUS INFECTION

(71) Applicant: Sanofi Pasteur, Lyon Cedex 07 (FR)

(72) Inventors: Jiansheng Yao, Toronto (CA); Yves Girerd-Chambaz, Messimy (FR); Isabelle Legastelois, Saint Andeol le Chateau (FR); Nathalie Mantel, Lyon (FR); Véronique Barban, Sainte-Foy-Les-Lyon (FR); Jean Lang, Mions (FR); Bruno Guy, Lyon (FR)

(21) Appl. No.: 14/416,492

(22) PCT Filed: Jul. 24, 2013
(86) PCT No.: PCT/EP2013/065669

§ 371 Date: Jan. 22, 2015

(30) Foreign Application Priority Data
Jul. 24, 2012 (EP) 12305908.1
Jul. 25, 2012 (EP) 12305911.5

(51) Int. Cl. A61K 39/12; C12N 7/00

(52) U.S. Cl. CPC A61K 39/12; C12N 7/00; C12N 2770/24134; A61K 2039/70

(57) ABSTRACT

The present invention relates to vaccine compositions that are useful in a method of protecting a human subject against dengue disease.

### FIELD OF THE INVENTION

The present invention relates to vaccine compositions and uses of such compositions in a method of protecting a human subject against dengue disease.

### BACKGROUND

Dengue is the second most important infectious tropical disease after malaria with approximately one-half of the world's population living in areas where there is a risk of epidemic transmission. There are estimated to be 50-100 million cases of dengue disease every year resulting in 500,000 patients being hospitalized for dengue hemorrhagic fever (DHF) and resulting in approximately 25,000 deaths.

Dengue disease infections are endemic in more than 100 tropical countries and dengue hemorrhagic fever (DHF) has been documented in 60 of these countries (Gubler, 2002, TRENDS in Microbiology, 10: 100-103).

Dengue disease is caused by four antigenically distinct, but closely related dengue virus serotypes of the flavivirus genus (Gubler et al., 1988, in: Epidemiology of arthropod-borne viral disease. Monath T P M, editor, Boca Raton (Fla.): CRC Press: 223-60; Kautner et al., 1997, J. of Pediatrics, 131: 516-524; Rigau-Perez et al., 1998, Lancet, 352: 971-977; Vaughn et al., 1997, J. Infect. Dis., 176: 322-30).

Dengue disease is usually transmitted by injection of the dengue virus during the blood meal of an Aedes aegypti mosquito infected by the virus. After an incubation period of 4-10 days, the illness begins abruptly and is followed by three phases: febrile (2 to 7 days), critical (24-48 hours—during which severe complications may occur) and recovery (48-72 hours). During the critical phase, life threatening complications such as hemorrhages, shock and acute organ impairment may occur. A proper management of these unpredictable outcomes can reduce the case fatality rate. Cure of dengue fever is complete after 7 to 10 days, but prolonged asthenia is normal. Reduced leukocyte and platelet numbers are frequently observed.

Dengue haemorrhagic fever (DHF) is a potentially deadly complication of dengue virus infection. DHF is characterized by a high fever and symptoms of dengue disease, but with extreme lethargy and drowsiness. Increased vascular permeability and abnormal homeostasis can lead to a decrease in blood volume, hypotension, and in severe cases, hypovolemic shock and internal bleeding. Two factors appear to play a major role in the occurrence of DHF—rapid viral replication with a high level of viremia (the severity of the disease being associated with the level of viremia; Vaughn et al., 2000, J. Inf. Dis., 181: 2-9) and a major inflammatory response with the release of high levels of inflammatory mediators (Rothman and Ennis, 1999, Virology, 257: 1-6; Alan L. Rothman. 2011, Nature Reviews Immunology, 11: 532-543)). The mortality rate for DHF can reach 10% without treatment, but is <1% in most centres with access to treatment.

Dengue shock syndrome (DSS) is a common progression of DHF and is frequently fatal. DSS results from generalized vasculitis leading to plasma leakage into the extravascular space. DSS is characterized by rapid and poor volume pulse, hypotension, cold extremities, and restlessness.

In Asia, DHF and DSS are observed primarily in children, with approximately 90% of those with DHF being less than 15 years of age (Malavige et al., 2004, Postgrad Med. J., 80: 588-601; Meulen et al., 2000, Trop. Med. Int. Health, 5:325-9). In contrast, outbreaks in the Caribbean and Central America have predominantly affected adults (Malavige et al., 2004, Postgrad Med. J., 80: 588-601).

The four serotypes of dengue virus possess approximately 60-80% sequence homology. Infection with one dengue serotype provides durable homologous immunity but limited heterologous immunity (Sabin, 1952, Am. J. Trop. Med. Hyg., 1: 30-50). Accordingly, an individual that has been infected with one serotype of dengue may subsequently become infected with a different serotype. In the past, it has been considered that a second infection arising from a different dengue virus serotype is theoretically a risk factor for the development of DHF, since the majority of patients that exhibit DHF have been previously exposed to at least one of the other four serotypes of dengue viruses.

To date, there is no specific treatment for dengue disease. Treatment for dengue disease is symptomatic, with bed rest, control of the fever and pain through antipyretics and analgesics, and adequate drinking. The treatment of DHF requires balancing of liquid losses, replacement of coagulation factors and the infusion of heparin.

Since dengue prevention measures, such as mosquito control and personal protection from bites, are limited in efficacy, difficult to enforce and expensive, a safe and efficacious dengue vaccine would be the best mode of prevention. However, there is no licensed vaccine of this type that is currently available.

It is therefore desirable to develop a vaccine composition that demonstrates efficacy when used in a method of protecting a human subject against dengue disease.

### SUMMARY OF THE INVENTION

The present invention relates to a dengue virus serotype 2 vaccine composition comprising:
(i) a dengue antigen selected from the group consisting of:
   (a) a live attenuated dengue virus;
   (b) an inactivated dengue virus;
   (c) a live attenuated or inactivated chimeric dengue virus;
   (d) a dengue virus-like particle (VLP); and
   (e) a combination of two or more of (a) to (d); or
(ii) a nucleic acid construct or viral vector which is able to express in a human cell a dengue antigen which is a dengue VLP;

wherein said dengue antigen comprises a polypeptide having at least 90% identity to SEQ ID NO: 12.

The present invention further relates to a vaccine composition comprising a dengue antigen of serotype 2 selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence encoding a protein comprising a polypeptide or polypeptides as defined in the claims.

A vaccine composition comprising a dengue antigen of serotype 2 selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence having at least 90% sequence identity to a sequence selected from the group consisting of the RNA equivalent of SEQ ID NO: 1, the RNA equivalent of SEQ ID NO: 4, the RNA equivalent of SEQ ID NO: 5, the RNA equivalent of SEQ ID NO: 6, the RNA equivalent of SEQ ID NO: 7 and SEQ ID NO: 25.

The present invention further relates to pharmaceutical formulation comprising a vaccine composition of the present invention and a pharmaceutically acceptable carrier, diluent or excipient.

The present invention further relates to a vaccine composition of the present invention for use in therapy.

The present invention further relates to a vaccine composition of the present invention for use in a method of protecting a human subject against dengue disease caused by a dengue virus of serotype 2.

The present invention further relates to a vaccine composition of the present invention for use in a method of generating neutralising antibodies against a dengue virus of serotype 2.

The present invention further relates to vaccine composition of the present invention which comprises a dengue antigen of serotype 1, a dengue antigen of serotype 2, a dengue antigen of serotype 3 and a dengue antigen of serotype 4 for use in a method of generating neutralising antibodies against the four serotypes of dengue.

The present invention further relates to the use of a vaccine composition of the present invention for the manufacture of a medicament for protecting a human subject against dengue disease caused by a dengue virus of serotype 2.

The present invention further relates to a method of protecting a human subject against dengue disease caused by a dengue virus of serotype 2, wherein said method comprises administering to said subject an effective amount of a composition according to the present invention.

The present invention further relates to a kit comprising a composition according to the present invention and instructions for the use of said composition in a method of protecting a human subject against dengue disease caused by a dengue virus of serotype 2.

The present invention relates to a vaccine composition for use in a method of protecting a human subject against dengue disease, wherein said composition comprises:
(i) a dengue antigen selected from the group consisting of:
   (a) a live attenuated dengue virus;
   (b) an inactivated dengue virus;
   (c) a live attenuated or inactivated chimeric dengue virus;
   (d) a dengue virus-like particle (VLP); and
   (e) a combination of two or more of (a) to (d); or
(iii) a nucleic acid construct or viral vector which is able to express in a human cell a dengue antigen which is a dengue VLP.

The present invention further relates to the use of a vaccine composition of the present invention for the manufacture of a medicament for protecting a human subject against dengue disease.

The present invention further relates to a method of protecting a human subject against dengue disease, wherein said method comprises administering to said human subject an effective amount of a composition according to the present invention.

Additionally, the present invention relates to a kit comprising a composition according to the present invention and instructions for the use of said composition in a method of protecting a human subject against dengue disease.

### DESCRIPTION OF THE FIGURE

FIG. 1 illustrates the construction of the YF-VAX cDNA by RT-PCR and cloning

### DEFINITIONS

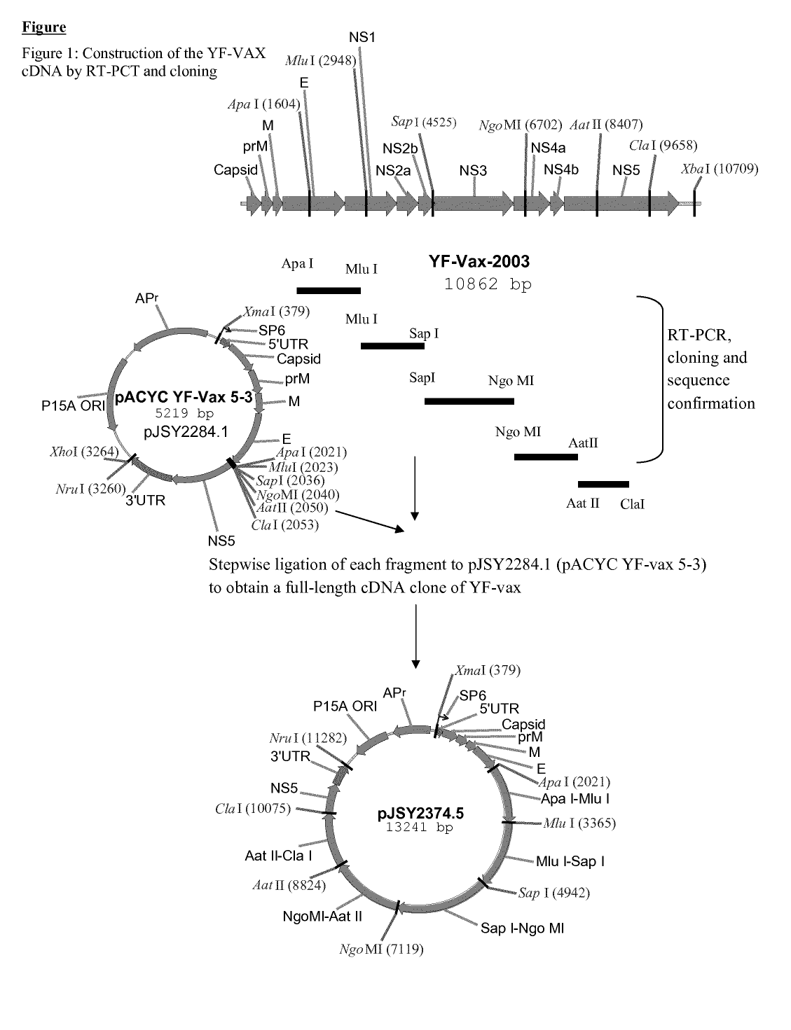

The term “Dengue disease”, as used herein, refers to the clinical symptoms exhibited by an individual following infection by any one of the four Dengue virus serotypes. Since 1970, clinical dengue has been classified according to World Health Organization guidelines as (i) dengue fever or (ii) dengue hemorrhagic fever (World Health Organization. Dengue hemorrhagic fever: Diagnosis, treatment, prevention and control 2^{nd} Ed. Geneva: WHO, 1997; ISBN 92 4 154500 3). In 2009, the WHO issued new guidelines that classify clinical dengue as (i) dengue with or without warning signs or (ii) severe dengue. Both classifications are shown in FIGS. 1 & 2 of Srikiatkachorn et al., Clin. Infect. Dis. (2011) 53(6): 563. According to the earlier classification, dengue fever is characterized by at least two symptoms selected from headache, arthralgia, retro-orbital pain, rash, myalgia, hemorrhagic manifestations, and leucopenia, together with supportive serology or occurrence at the same location and time as other confirmed dengue cases. Progression to Dengue hemorrhagic fever is confirmed when fever, hemorrhagic manifestations, thrombocytopenia and evidence of plasma leakage are all observed. According to the more recent classification, diagnosis of dengue requires the presence of fever and at least two clinical symptoms selected from nausea, vomiting, rash, aches and pains, a positive tourniquet test, or any warning signs selected from abdominal pain and tenderness, persistent vomiting, clinical fluid accumulation, mucosal bleed, lethargy or restlessness, liver enlargement greater than 2 cm or an increase in hematocrit concurrent with a rapid decrease in platelet count. Severe dengue is diagnosed when any of the following events are observed: severe plasma leakage leading to shock or respiratory distress, severe bleeding as evaluated by clinicians or severe organ involvement

The term “Dengue hemorrhagic fever or DHF”, as used herein, refers to virologically-confirmed dengue disease wherein fever, hemorrhagic manifestations, thrombocytopenia and evidence of plasma leakage are all observed. DHF, as used herein, may be further defined on the basis of its severity. For instance, DHF may be defined as being of Grade I, Grade II, Grade III or Grade IV (World Health Organization. Dengue hemorrhagic fever: Diagnosis, treatment, prevention and control 2^{nd} Ed. Geneva: WHO, 1997; ISBN 92 4 154500 3). Grade I is defined as fever accompanied by non-specific constitutional symptoms; the only haemorrhagic manifestation is a positive tourniquet test and/or easy bruising. Grade II is defined as spontaneous bleeding in addition to the manifestations of Grade I patients, usually in the form of skin or other haemorrhages. Grade III is defined as circulatory failure manifested by a rapid, weak pulse and narrowing of pulse pressure or hypotension, with the presence of cold clammy skin and restlessness. Grade IV is defined as profound shock with undetectable blood pressure or pulse. As would be understood by a person of skill in the art, in the practice of the present invention, e.g. a method of protecting against DHF, said DHF need not be virologically-confirmed.

The term “virologically-confirmed dengue”, as used herein, refers to an acute febrile episode which is confirmed to be induced by a dengue virus, e.g. by reverse transcriptase polymerase chain reaction (RT-PCR) or by a dengue non-structural 1 (NS1) protein enzyme-linked immunosorbent assay (ELISA). In the RT-PCR method, serum samples are tested according to the method of Callahan et al, J. Clin. Microbiol. (2001) 39: 4119. Briefly, RNA is extracted from the serum to discard potential Taq polymerase inhibitors or interfering factors, using a commercial kit. Then an RT-PCR reaction is carried out with serotype specific primers from the dengue NS5 gene sequence. Results are expressed as a concentration of log₁₀GEQ (genome equivalent)/mL, by comparison with standards containing known concentrations of viral genomic serotype-specific nucleic acid sequences integrated into plasmids. In the ELISA method, 50 μL of patient serum, a positive control, a negative control, or a cut-off control are diluted 1:2 in sample diluent and combined with 100 μL of diluted horseradish peroxidase (HRP)-labeled anti-NS1 monoclonal Ab (MAb). The diluted serum and conjugate are added to capture anti-NS1 MAb-coated microwells, and plates are incubated for 90 minutes at 37° C. Capture MAb/NS1/HRP-labeled-MAb complexes are formed when NS1 is present in the serum. Complexes are detected via a colorimetric reaction in positive wells which is induced by adding 160 μL of 3,3′,5,5′ tetramethylbenzidine (TMB) substrate and incubating for 30 minutes at room temperature in the dark. The reaction is stopped with the addition of 100 μL of stop solution (1N H₂SO4) and the plate is read. A sample ratio is determined for each sample by dividing the average optical density (OD) of the test sample by the average OD of the cut-off control (tested in quadruplicate). Sample ratios of <0.5, 0.5-<1.0, and ≧1 are indicative of negative, equivocal, and positive results, respectively.

The term “severe virologically-confirmed dengue”, as used herein, refers to dengue haemorrhagic fever (DHF) as defined by the 1997 WHO classification and further characterized by the following additional list of symptoms: haemorrhage requiring blood transfusion, objective evidence of capillary permeability, signs of circulatory failure or visceral manifestations.

The term “dengue shock syndrome”, as used herein, refers to the most severe complications of DHF as defined above. According to the 1997 WHO classification, DSS corresponds to DHF of Grades III and IV.

The term “dengue fever viruses”, “dengue viruses” and “DEN” are used interchangeably. They refer to positive single-strand RNA viruses belonging to the Flavivirus genus of the family of flaviviridae. There are four different serotypes of dengue virus (serotypes 1, 2 3 and 4), which possess approximately 60-80% sequence homology. The organization of the genome comprises the following elements: a 5′ non-coding region (NCR), a region encoding structural proteins (capsid (C), pre-membrane (prM) and envelope (E)) and a region encoding non-structural proteins (NS1-NS2A-NS2B-NS3-NS4A-NS4B-NS5) and a 3′ NCR. The dengue viral genome encodes an uninterrupted coding region which is translated into a single polyprotein which undergoes post-translational processing. The sub-sequences included in the prM-E sequences may be numbered in various ways: (i) the total prM-E protein sequence is numbered from position 1 to position 661, with the preM protein sequence designated as position 1 to position 90/91, the M protein sequence designated as position 91/92 to position 166 and the E protein sequence designated as position 167 to position 661; (ii) the prM and M protein sequences are numbered together, i.e. from position 1 to position 166 of the total sequence and E is numbered separately from position 1 to position 495; (iii) the prM, M and E sequences are numbered separately, i.e. prM is numbered from position 1 to 90/91, M is numbered from 1 to 75/76 and E from position 1 to position 495. In the present disclosure the E protein is always numbered from position 1 to position 495. For example, a residue designated herein as E-154 refers to position 154 of the E protein.

In the context of the present invention, “vaccinal dengue virus” refers to a virus which is capable of inducing neutralizing antibodies against the dengue virus serotype from which the vaccinal dengue virus is derived, by the administration of such vaccinal dengue virus to an immunocompetent subject. Examples of vaccinal dengue viruses which may be used in a method of the present invention include inactivated dengue viruses, live attenuated dengue viruses and live attenuated or inactivated chimeric dengue viruses. Serotypes of vaccinal dengue viruses for use in the present invention include serotypes 1, 2, 3, and 4. Preferably a vaccinal dengue virus for use in the present invention is a live attenuated chimeric dengue virus.

The expression “inactivated virus”, as used herein, refers to a virus that is incapable of replication to any significant degree in cells permissive for replication of the corresponding wild type virus. Viruses may be inactivated by a number of means well known to those skilled in the art. Examples of methods for inactivating a virus include chemical treatments, or radiation treatments (including heat or electromagnetic radiation typically in the forms of X-ray or ultraviolet radiation).

The term “inactivated dengue virus”, as used herein refers to an inactivated wild-type virus containing all the dengue structural proteins (env, premembrane/membrane and capsid proteins) and inactivated viral RNA. An inactivated dengue virus may also refer to an inactivated chimeric dengue virus. Inactivated dengue viruses are for instance described in U.S. Pat. No. 6,254,873.

The term “live attenuated virus or LAV”, as used herein, refers to a virus which is not able to induce a disease state characterised by the same sets of symptoms associated with the corresponding wild-type virus. Examples of live attenuated viruses are well known in the art. A live attenuated virus may be prepared from a wild-type virus, for example, by recombinant DNA technology, site directed mutagenesis, genetic manipulation, serial passages on replication-competent cells, chemical mutagenesis treatment or electromagnetic radiation.

The term “live attenuated dengue virus”, as used herein, refers to a live dengue virus derived from a virulent wild-type dengue virus by genetic modification resulting in attenuation of virulence and an inability to induce a disease state characterised by the same sets of symptoms associated with the corresponding wild type dengue virus. Examples of live attenuated dengue viruses useful in the practice of the present invention include VDV-1, VDV-2, and the strains described for example in applications WO 02/66621, WO 00/57904, WO 00/57908, WO 00/57909, WO 00/57910, WO 02/0950075 and WO 02/102828. Live attenuated dengue viruses of serotype 1 which may be used in the method of the invention include VDV-1. Live attenuated dengue viruses of serotype 2 which may be used in the method of the invention include VDV-2, and LAV-2.

“VDV” and “Vero dengue vaccine” are used interchangeably herein and designate a live attenuated dengue virus capable of replication in Vero cells and capable of inducing a specific humoral response, including the induction of neutralizing antibodies, in a human.

The DEN-1 16007/PDK13 strain, also called “LAV1”, is derived from wild-type DEN-1 (dengue virus serotype 1) 16007 strain which has undergone 11 passages through primary dog kidney (PDK) cells (DEN-1 16007/PDK11). LAV1 has been described in patent application EP1 159968 in the name of Mahidol University and has been filed with the National Microorganisms Cultures Collection (CNCM) under number I-2480. “VDV-1” is a virus derived from LAV1 by subsequent adaptation to Vero cells; in this regard, the RNA from LAV1 has been extracted and purified before being transfected into Vero cells. The VDV-1 strain has subsequently been obtained by plate purification and amplification in Vero cells. The VDV-1 strain has 14 additional mutations in comparison with the DEN-1 16007/PDK13 strain (13 passes through PDK cells). A process for preparing and characterizing the VDV-1 strain has been described in international patent application filed under number WO06/134433 in the names of Sanofi-Pasteur and the Center for Disease Control and Prevention.

The DEN-2 16681/PDK53 strain, also known as “LAV2”, has been obtained from wild-type strain DEN-2 (dengue virus serotype 2) 16681 which has undergone 50 passes through PDK cells (DEN-2 16681/PDK50). LAV2 has been described in in patent application EP1159968 in the name of Mahidol University and has been filed with the National Microorganisms Cultures Collection (CNCM) under number 1-2481. “VDV-2” is a strain derived from LAV2 by subsequent adaptation to Vero cells; in this regard, the RNA from LAV2 has been extracted and purified before being transfected in Vero cells. The VDV-2 strain has subsequently been obtained by plate purification and amplification in Vero cells. The VDV-2 strain has 10 additional mutations in comparison with the DEN-2 16681/PDK53 strain (53 passes through PDK cells), including 4 silent mutations. A process for preparing and characterizing the VDV-2 strain has been described in the international patent application filed under number WO06/134443 in the names of Sanofi-Pasteur and the Center for Disease Control and Prevention. The complete nucleic acid sequence of the VDV-2 strain is as shown in SEQ ID NO: 24. The sequence of the E protein of the VDV-2 strain is as shown in SEQ ID NO: 26 and the sequence of the M protein of the VDV-2 strain is as shown in the SEQ ID NO: 27.

The VDV 1 and 2 strains are prepared by amplification in Vero cells. The viruses produced are harvested and clarified from cell debris by filtration. The DNA is digested by treatment with enzymes. Impurities are eliminated by ultrafiltration. Infectious titers may be increased by a concentration method. After adding a stabilizer, the strains are stored in lyophilized or frozen form before use and then reconstituted when needed.

In the context of the invention, “dengue chimera or chimeric dengue virus” means a recipient flavivirus in which the genetic backbone has been modified by exchanging the sequences encoding the prM and E proteins of the recipient flavivirus by the corresponding sequences of a dengue virus. Typically, the recipient flavivirus may be attenuated. The recipient flavivirus may be a yellow fever (YF) virus such as the attenuated YF 17D, YF 17DD and YF 17D204 (YF-VAX®) viruses; in that case, such chimeras are referred to as YF/dengue chimeras. The recipient flavivirus may also be a dengue virus and in that case, it is referred to as dengue/dengue chimera, the dengue virus serotype characteristic of the prM and E proteins being identical or different from the recipient dengue virus serotype characteristic of the genetic backbone. When the serotypes are identical, the recipient dengue virus and the dengue virus from which the prM and E protein encoding sequences originate, are two different virus strains of the same serotype. For use in the present invention, chimeric dengue viruses are typically YF/dengue chimeras. Chimeric dengue viruses are preferably inactivated or live attenuated chimeric dengue viruses. Advantageously, the recipient flavivirus of a live attenuated chimeric dengue virus of the present invention is YF 17D or YF 17D204 (YF-VAX®). According to one embodiment dengue chimera is an inactivated virus. According to an alternative embodiment the dengue chimera is a live attenuated virus. Dengue Chimera that can be used in a vaccine composition of the present invention include Chimerivax™ Dengue Serotype 1 (also known as CYD-1), Chimerivax™ Dengue Serotype 2 (also known as CYD-2), Chimerivax™ Dengue Serotype 3 (also known as CYD-3) and Chimerivax™ Dengue Serotype 4 (also known as CYD-4).

Examples of chimeric dengue viruses useful in the practice of the present invention include the dengue/YF chimeric viruses described in patent application WO 98/37911 and dengue/dengue fever chimeras such as those described in patent applications WO 96/40933 and WO 01/60847.

In one embodiment, the chimeric YF/dengue virus comprises the genomic backbone of the attenuated yellow fever virus strain YF17D (Theiler M. and Smith H. H., 1937, J. Exp. Med., 65. 767-786), e.g. viruses YF17D/DEN-1, YF17D/DEN-2, YF17D/DEN-3 and YF17D/DEN-4. Examples of YF17D strains which may be used include YF17D204 (YF-VAX®, Sanofi-Pasteur, Swiftwater, Pa., USA; Stamaril®, Sanofi-Pasteur, Marcy l'Etoile, France; ARILVAX™, Chiron, Speke, Liverpool, UK; FLAVIMUN®, Berna Biotech, Bern, Switzerland; YF17D-204 France (X15067, X15062); YF17D-204,234 US (Rice et al., 1985, Science, 229: 726-733), or the related strains YF17DD (Genbank access number U17066), YF17D-213 (Genbank access number U17067) and the strains YF17DD described by Galler et al. (1998, Vaccines, 16(9/10): 1024-1028). In another embodiment, the chimeric YF/dengue virus comprises the genomic backbone of the attenuated yellow fever virus strain YF17D204 (YF-VAX®).

One example of a chimeric dengue virus particularly suitable for use in the practice of the present invention is a “Chimerivax dengue virus”. As used herein, a “Chimerivax dengue virus”, is a live attenuated chimeric YF/dengue virus which comprises the genomic backbone of a YF17D or YF17D204 (YF-VAX®) virus in which the nucleic acid sequences encoding the pre-membrane (prM) and envelope (E) proteins have been replaced by nucleic acid sequences encoding the corresponding structural proteins of a dengue virus. A preferred chimeric dengue virus for use in the present invention is a live attenuated chimeric YF/dengue virus which comprises the genomic backbone of a YF17D virus in which the nucleic acid sequences encoding the pre-membrane (prM) and envelope (E) proteins have been replaced by nucleic acid sequences encoding the corresponding structural proteins of a dengue virus. A preferred chimeric dengue virus for use in the present invention is a live attenuated chimeric YF/dengue virus which comprises the genomic backbone of a YF17D204 (YF-VAX®) virus in which the nucleic acid sequences encoding the pre-membrane (prM) and envelope (E) proteins have been replaced by nucleic acid sequences encoding the corresponding structural proteins of a dengue virus. Construction of such Chimerivax viruses may be achieved in accordance with, or in substantial accordance with, the teaching of Chambers, et al. (1999, J. Virology 73(4):3095-3101). The particular Chimerivax (CYD) dengue viruses described in the examples have been generated by using prM and E sequences from strains DEN 1 PU0359 (TYP1 140), DEN2 PU0218, DEN3 PaH881/88 and DEN 4 1228 (TVP 980) and the genomic backbone of YF17D virus. Those particular Chimerivax strains are referred to herein (see the present examples) as “CYD-1”, “CYD-2”, “CYD-3” and “CYD-4” respectively. The preparation of these particular CYD-1, CYD-2, CYD-3 and CYD-4 strains has been described in detail in international patent applications WO 98/37911, WO 03/101397, WO 07/021672, WO 08/007021, WO 08/047023 and WO 08/065315, to which reference may be made for a precise description of the processes for their preparation. Alternatively, other dengue fever virus strains may be used as a source of nucleic acids to facilitate construction of chimeric viruses useful in the practice of the present invention, for example in the construction of other Chimerivax dengue serotype 1 (CYD-1), Chimerivax dengue serotype 2 (CYD-2), Chimerivax dengue serotype 3 (CYD-3) and Chimerivax dengue serotype 4 (CYD-4) strains. Advantageously, a vaccine composition of the present invention, e.g. a chimeric dengue virus, of serotype 2 may comprise prM-E sequences having at least 90%, at least 95%, at least 98% or at least 99% identity to the prM-E sequences from the serotype 2 strains LAV-2, BID-V585, PR/DB023 or MD1280 as described in the examples or may comprise prM-E sequences having at least 90%, at least 95%, at least 98% or at least 99% identity to the prM-E sequence shown in SEQ ID NO: 2. Advantageously, a vaccine composition, e.g. a chimeric dengue virus, of serotype 2 for use in the method of the present invention may comprise prM-E sequences from the serotype 2 strains LAV-2, BID-V585, PR/DB023 or MD1280 or the prM-E sequence from SEQ ID NO: 2 as described in the examples. When the recipient genomic backbone of such chimeric dengue viruses is derived from YF-VAX®, such strains are referred to herein as CYD-LAV, CYD-BID, CYD-PR and CYD-MD. A vaccine composition of the present invention comprising chimeric dengue virus of serotype 2 generated using the prM-E sequences of the serotype 2 strains LAV-2 (SEQ ID NO: 8), BID-V585 (SEQ ID NO: 9), PR/DB023 (SEQ ID NO: 10), MD1280 (SEQ ID NO: 11) or SEQ ID NO: 2, or generated using prM-E sequences having at least 90%, at least 95%, at least 98% or at least 99% identity to the prM-E sequences from the serotype 2 strains LAV-2, BID-V585, PR/DB023, MD1280 or the prM-E sequence from SEQ ID NO: 2 may advantageously be used in combination with CYD-1, CYD-3 and CYD-4 in a vaccine composition according to the present invention. Examples of chimeric dengue virus of serotype 2 generated using the prM-E sequences of the serotype 2 strains LAV-2 (SEQ ID NO: 8), PR/DB023 (SEQ ID NO: 10) and MD1280 (SEQ ID NO: 11) include CYD-LAV, CYD-PR and CYD-MD respectively.

An alternative embodiment of chimeric dengue virus usable in the method of protection of the invention is a recipient flavivirus in which the genetic backbone has been modified by exchanging (i) the sequence encoding the E protein of the recipient flavivirus by the corresponding sequence of a dengue virus and (ii) the sequence encoding the prM protein of the recipient flavivirus by the corresponding sequence of a non-dengue flavivirus, e.g. a JEV virus. Typically, the said chimeric virus may be a live attenuated virus or an inactivated virus. Examples of such chimeric dengue viruses are described in WO2011/138586.

A vaccinal dengue virus of serotype 1 for use in a vaccine composition of the present invention may, for example, be the strain VDV1, CYD-1 or a YF17D/DEN-1 chimeric virus comprising the prM and E amino acid sequences of the DEN-1 16007/PDK13 strain. A vaccinal dengue virus of serotype 2 for use in the method of the present invention may, for example, be the strain VDV2, CYD-2, a YF17D/DEN-2 chimeric virus comprising the prM and E amino acid sequences of the DEN-2 16681/PDK53 strain, a chimeric virus comprising the prM and E amino acid sequences of the DEN-2 strains LAV-2, BID-V585, PR/DB023 or MD1280 or a chimeric virus comprising prM-E sequences having at least 90%, at least 95%, at least 98% or at least 99% identity to the prM-E sequences from the serotype 2 strains LAV-2, BID-V585, PR/DB023 or MD1280 or at least 90%, at least 95%, at least 98% or at least 99% identity to the prM-E sequence in SEQ ID NO: 2. A vaccinal dengue virus of serotype 3 for use in the method of present invention may, for example, be CYD-3 or an alternative YF17D/DEN-3 chimeric virus. An example of a vaccinal dengue virus of serotype 4 is CYD-4 or an alternative YF17D/DEN-4 chimeric virus.

A composition of the present invention comprises at least one dengue antigen. Typically a composition of the present invention comprises a dengue antigen, e.g. a vaccinal dengue virus, of each of serotypes 1, 2, 3 and 4. Dengue antigens, e.g. vaccinal dengue viruses, of the present invention of each serotype may be as described herein. For instance, a composition of the present invention may advantageously comprise any one of the following combinations of dengue antigens: i) a dengue antigen comprising the prM and E sequences of CYD-1, a dengue antigen comprising the prM and E sequences of CYD-LAV, a chimeric dengue virus comprising the prM and E amino acid sequences of CYD-3 and a dengue antigen comprising the prM and E sequences of CYD-4; ii) a dengue antigen comprising the prM and E sequences of CYD-1, a dengue antigen comprising the prM and E sequences of CYD-BID, a dengue antigen comprising the prM and E sequences of CYD-3 and a dengue antigen comprising the prM and E sequences of CYD-4; (iii) a dengue antigen comprising the prM and E sequences of CYD-1, a dengue antigen comprising the prM and E sequences of CYD-PR, a dengue antigen comprising the prM and E sequences of CYD-3 and a dengue antigen comprising the prM and E sequences of CYD-4; (iv) a dengue antigen comprising the prM and E sequences of CYD-1, a dengue antigen comprising the prM and E sequences of CYD-MD, a dengue antigen comprising the prM and E sequences of CYD-3 and a dengue antigen comprising the prM and E sequences of CYD-4;. For instance, a composition of the present invention may also advantageously comprise any one of the following combinations of dengue antigens: i) CYD-1, CYD-LAV, CYD-3 and CYD-4; ii) CYD-1, CYD-BID, CYD-3 and CYD-4; (iii) CYD-1, CYD-PR, CYD-3 and CYD-4 or (iv) CYD-1, CYD-MD, CYD-3 and CYD-4. A composition of the present invention may also advantageously comprise the following combination of dengue antigens: i) a dengue antigen comprising the prM and E sequences of CYD-1, VDV2, a dengue antigen comprising the prM and E sequences of CYD-3 and a dengue antigen comprising the prM and E sequences of CYD-4. For instance, a composition of the present invention may advantageously comprise CYD-1, VDV-2, CYD-3 and CYD-4. A composition of the present invention, as described herein, may advantageously comprise a dengue antigen of serotype 2 which comprises the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. A composition of the present invention, as described herein, may advantageously comprise a dengue antigen of serotype 2 which comprises a sequence having at least 90% identity to the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. For example, said sequence may be at least 91%, at least 92%, at least 93%, at least 94%, at least 95%, at least 96%, at least 97%, at least 98% or at least 99% identical to the prm-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2.

The term “virus-like particles or VLPs”, as used herein, refers to virus particles that do not contain replicative genetic material but present at their surface a dengue E protein in a repetitive ordered array similar to the virion structure. Typically, dengue VLPs also contain dengue prM and/or M, and E proteins. VLPs may be produced in vitro (Zhang et al, J. Virol. (2011) 30 (8):333). VLPs may also be produced in vivo. To that end, nucleic acid constructs (e.g. DNA or RNA constructs) encoding prM and E dengue proteins may be introduced into a cell of a subject, e.g. a human subject, via methods known in the art, e.g. via use of a viral vector. Any viral vector may be used provided it is able to contain and express both prM and E dengue virus sequences. Non-limiting examples of viral vectors that may be used in the method of the present invention include the poxviruses (e.g. the attenuated pox Ankara virus) and the measles virus. For use in the present invention, a particular category of viral vector expressing VLPs in vivo includes replication-deficient pseudoinfectious (PIV) viruses, e.g. according to the Replivax™ technology. (Rumyantsev A A, et al. Vaccine. 2011 Jul. 18; 29(32):5184-94).

The term “replication-defective pseudo-infectious virus”, as used herein, refers to a virion particle that is replication-defective in vivo, owing to the absence in their genome of an essential sequence of the replicative cycle, for example the sequence encoding a capsid protein. However, the virion particles can propagate in a culture of helper cells that provide for the essential sequence(s) in trans. Replication-deficient pseudoinfectious viruses for use in the present invention include any virus according to the above definition which is capable of expressing the prM and E proteins of a dengue virus of any serotype. Examples include replication defective flavivirus/dengue chimeras such as replication defective West Nile virus/dengue, Japanese Encephalitis virus/dengue and YF/dengue chimeras.

The ability of a vaccine composition of the present invention to provoke an immune response in a subject (i.e. induce the production of neutralizing antibodies) can be assessed, for example, by measuring the neutralizing antibody titre raised against the dengue virus serotype(s) comprised within the composition. The neutralizing antibody titre may be measured by the Plaque Reduction Neutralization Test (PRNT₅₀) test. Briefly, neutralizing antibody titre is measured in sera collected from vaccinated subjects at least 28 days following administration of a vaccine composition of the present invention. Serial, two-fold dilutions of sera (previously heat-inactivated) are mixed with a constant challenge-dose of each dengue virus of serotype 1, 2, 3 or 4 as appropriate (expressed as PFU/mL). The mixtures are inoculated into wells of a microplate with confluent Vero cell monolayers. After adsorption, cell monolayers are incubated for a few days. The presence of dengue virus infected cells is indicated by the formation of infected foci and a reduction in virus infectivity due to the presence of neutralising antibodies in the serum samples can thus be detected. The reported value (end point neutralization titre) represents the highest dilution of serum at which ≧50% of dengue challenge virus (in foci counts) is neutralized when compared to the mean viral focus count in the negative control wells (which represents the 100% virus load). The end point neutralization titres are presented as continuous values. The lower limit of quantification (LLOQ) of the assay is 10 (1/dil). It is commonly considered that seroconversion occurs when the titer is superior or equal to 10 (1/dil). As PRNT tests may slightly vary from a laboratory to another the LLOQ may also slightly vary. Accordingly, in a general manner, it is considered that seroconversion occurs when the titre is superior or equal to the LLOQ of the test. Neutralising antibody titres were considered in the following references, but the authors did not establish a correlate of protection (Guirakhoo et al, J. Virol. (2004) 78 (9): 4761; Libraty et al, PLoS Medicine (2009) 6 (10); Gunther et al, Vaccine (2011) 29: 3895) and Endy et al, J. Infect. Dis. (2004), 189(6): 990-1000).

The term “CCID₅₀” refers to the quantity of virus (e.g. vaccinal virus) infecting 50% of the cell culture. The CCID₅₀ assay is a limit dilution assay with statistical titer calculation (Morrison D et al J Infect Dis. 2010; 201(3):370-7)).

The term “human subject” is intended to mean males and females of various ages. Preferably a human subject according to the present invention is less than 18 years of age or less than 12 years of age. For example, a human subject according to the present invention may be 0-17 years of age, 0-11 years of age, 4-17 years of age, 4-11 years of age, 4-6 years of age, 6-8 years of age, 8-10 years of age, 2-8 years of age, 2-11 years of age, 2-14 years of age, 9-16 years of age, 12-17 years of age or 18-45 years of age. More preferably, a human subject according to the present invention is 4-11 years of age, 2-14 years of age or 9-16 years of age. A human subject according to the present invention may be at least 9 months old or less than 9 months old. For instance a human subject according to the present invention may be 9 months to 16 years of age, 9 months to 14 years of age, 9 months to 11 years of age or 9 months to 8 years of age. A human subject according to the present invention may be at least 9 months old, with no history of severe allergy to any component of the vaccine composition as defined herein, no congenital or acquired immune deficiency, no symptomatic HIV infection and said subject should not be pregnant or breast feeding.

As used herein, the expression “flavivirus-naïve subject” refers to a subject who has not been infected by a flavivirus nor previously immunized with a flavivirus vaccine, i.e. a serum sample taken from said subject will produce a negative result in a flavivirus ELISA or PRNT assay.

As used herein, the expression “dengue-naïve subject” refers to a subject who has not been infected by a dengue virus nor previously immunized with a dengue vaccine, i.e. a serum sample taken from said subject will produce a negative result in a dengue ELISA or PRNT assay.

As used herein, the expression “flavivirus-immune subject” refers to a subject who has been infected or immunized by a flavivirus before administration of the vaccine composition of the invention, i.e. a serum sample taken from said subject will produce a positive result in a flavivirus ELISA or PRNT assay.

As used herein, the expression “dengue-immune subject” refers to a subject who has been infected by a dengue virus or immunized by a dengue vaccine before administration of the vaccine composition of the present invention, i.e. a serum sample taken from said subject will produce a positive result in a dengue ELISA or PRNT assay.

In accordance with the present invention, a “method of protecting”, as used herein, results in a reduction in the severity or in the likelihood of developing dengue disease in a human subject exposed to a dengue virus. Advantageously, said reduction is statistically significant. For example, a method of protecting, according to the present invention, may result in a reduction in at least one symptom of dengue disease as defined herein or a reduction in a combination of any two or more of those symptoms. The protection may result in any one or more of the following:
(i) a statistically significant reduction in the incidence or likelihood of, e.g. the prevention of, symptomatic virologically-confirmed dengue disease caused by dengue virus of any serotype;
(ii) a statistically significant reduction in the incidence or likelihood of, e.g. the prevention of, symptomatic virologically-confirmed dengue disease caused by dengue virus of any one of serotypes 1, 3 or 4;
(iii) a statistically significant reduction in the incidence or likelihood of, e.g. the prevention of, symptomatic dengue disease caused by dengue virus of any serotype;
(iv) a statistically significant reduction in the incidence or likelihood of, e.g. the prevention of, symptomatic dengue disease caused by dengue virus of any one of serotypes 1, 3 or 4;
(v) a statistically significant reduction in the incidence or likelihood of, e.g. the prevention of, severe virologically-confirmed dengue caused by dengue virus of any serotype;
(vi) a statistically significant reduction in the incidence or likelihood of, e.g. the prevention of, severe dengue disease caused by dengue virus of any serotype;
(vii) a statistically significant reduction in the incidence or likelihood of, e.g. the prevention of, dengue hemorrhagic fever cases of Grades I to IV caused by dengue virus of any serotype;
(viii) a statistically significant reduction in the incidence or likelihood of, e.g. the prevention of, DHF cases of Grade I caused by dengue virus of any serotype;
(ix) a statistically significant reduction in the incidence or likelihood of, e.g. the prevention of, DHF cases of Grade II caused by dengue virus of any serotype;
(x) a statistically significant reduction in the incidence or likelihood of, e.g. the prevention of, DHF cases of Grade III caused by dengue virus of any serotype;
(xi) a statistically significant reduction in the incidence or likelihood of, e.g. the prevention of, DHF cases of Grade IV caused by dengue virus of any serotype;
(xii) a statistically significant reduction in the incidence or likelihood of, e.g. the prevention of, fever or a reduction in the mean duration and/or intensity of fever;
(xiii) a statistically significant reduction in the incidence or likelihood of, e.g. the prevention of, plasma leakage as defined by a change in haematocrit or a reduction in the mean value for plasma leakage as defined by a change in haematocrit;
(xiv) a statistically significant reduction in the incidence or likelihood of, e.g. the prevention of, thrombocytopenia or a reduction in the mean value for thrombocytopenia;
(xv) a statistically significant reduction in the incidence or likelihood of, e.g. the prevention of, increases in the level of liver enzymes including alanine aminotransferase (ALT) and aspartate aminotransferase (AST);
(xvi) a statistically significant reduction in the incidence or likelihood of, e.g. the prevention of, hospitalization due to virologically-confirmed dengue disease caused by dengue virus of any serotype;
(xvii) a statistically significant reduction in the incidence or likelihood of, e.g. the prevention of, hospitalization due to dengue disease caused by dengue virus of any serotype;
(xviii) a statistically significant reduction in the length of hospital stay due to virologically-confirmed dengue disease.
(xix) a statistically significant reduction in the length of hospital stay due to dengue disease.

The duration and intensity of fever are monitored and recorded according to standard hospital procedures. In a human subject, a fever (i.e. a febrile episode) is defined as the observance of two temperature readings of at least 37.5° C. measured twice over an interval of at least 4 hours. Measurement of haematocrit, thrombocytopenia and hepatic enzyme levels are standard tests well-known to the person of skill in the art, for example as described in the pharmacopea.

Protection against dengue disease, for example as defined in points (i) to (xix) above, may be demonstrated in respect of dengue disease caused by a particular dengue virus serotype. For example, protection against dengue disease, as defined herein, may be demonstrated in respect of dengue disease caused by a dengue virus of serotype 1, a dengue virus of serotype 2, a dengue virus of serotype 3 or a dengue virus of serotype 4. Advantageously, protection against dengue disease, as defined herein, may be demonstrated in respect of dengue disease caused by, for example, dengue virus of serotype 1 or serotype 3, dengue virus of serotype 1 or serotype 4, dengue virus of serotype 3 or serotype 4, dengue virus of serotype 1 or serotype 2, dengue virus of serotype 2 or serotype 3, dengue virus of serotype 2 or serotype 4, dengue virus of serotype 1, 2 or 3, dengue virus of serotype 1, 3 or 4, dengue virus of serotype 2, 3 or 4 or dengue virus of serotype 1, 2, 3 or 4.

Protection against dengue disease, as defined herein, may advantageously be demonstrated in particular sub-groups of human subjects. For instance, protection against dengue disease may advantageously be demonstrated in a human subject who is less than 18 years of age or less than 12 years of age. For example, a human subject according to the present invention may be 0-17 years of age, 0-11 years of age, 4-17 years of age, 4-11 years of age, 4-6 years of age, 6-8 years of age, 8-10 years of age, 2-8 years of age, 2-11 years of age, 2-14 years of age, 9-16 years of age, 12-17 years of age or 18-45 years of age. More preferably, a human subject according to the present invention is 4-11 years of age, 2-14 years of age or 9-16 years of age. A human subject according to the present invention may be at least 9 months old or less than 9 months old. For instance a human subject according to the present invention may be 9 months to 16 years of age, 9 months to 14 years of age, 9 months to 11 years of age or 9 months to 8 years of age. A human subject according to the present invention may be at least 9 months old, with no history of severe allergy to any component of the vaccine composition as defined herein, no congenital or acquired immune deficiency, no symptomatic HIV infection and said subject should not be pregnant or breast feeding.

Protection against dengue disease, as defined herein, may advantageously be demonstrated in particular countries, areas or regions of the world. For instance, protection against dengue disease may advantageously be demonstrated in a dengue endemic area. For instance, a dengue endemic area according to the present invention in which protection may be demonstrated may comprise those American countries or parts thereof which fall within the tropics and sub-tropics. A dengue endemic area in which protection may be demonstrated according to the present invention may thus comprise any one or more of the following: Brazil, Venezuela, Colombia, Ecuador, Peru, Bolivia, Paraguay, Panama, Costa Rica, Nicaragua, Honduras, El Salvador, Guatemala, Belize, Mexico, the USA and the islands of the Caribbean. In a particular embodiment, a dengue endemic area of the present invention in which protection may be demonstrated may consist of the following: Brazil, Colombia, Honduras, Mexico and Puerto Rico. A dengue endemic area in which protection may be demonstrated according to the present invention may also include south Asian and Oceania countries within the tropics and sub-tropics. A dengue endemic area according to the present invention in which protection may be demonstrated may thus consist of any one or more of the following: India, Myanmar (Burma), Thailand, Laos, Vietnam, Cambodia, Indonesia, Malaysia, Singapore, the Philippines, Taiwan, Papua New Guinea and Australia. In a dengue endemic area in which protection may be demonstrated according to the present invention, a particular serotype, strain or genotype of wild type dengue virus may be the dominant circulating strain. For example, a dengue virus of serotype 2 may be characterised as having an Asian I or an Asian/American genotype. Asian/American genotype strains are characterised by at least one of, at least two of, at least three of, at least four of, at least five of or all six of the following residues Arg, Asn, Asp, Thr, Gly and His at positions prM-16, E-83, E-203, E-226, E-228 and E-346 respectively (wherein prM-16 designates position 16 of the prM protein and E-83 etc. designates position 83 of the E protein). Asian I genotype strains are characterised by at least one of, at least two of, at least three of, at least four of, at least five of or all six of the following residues Ile, Lys, Asn, Arg, Glu and Tyr at positions prM-16, E-83, E-203, E-226, E-228 and E-346 respectively (see Table 1 of Hang et al., PLoS NTD, 4(7): e757). A preferred dengue endemic area in which protection may be demonstrated according to the present invention is one in which a dengue virus having an Asian/American genotype is the dominant circulating strain, i.e. at least 50%, at least 60%, at least 70%, at least 80%, at least 90%, at least 95% or 100% of the cases of dengue disease in said dengue endemic area are caused by dengue virus having an Asian/American genotype. A preferred dengue endemic area in which protection may be demonstrated according to the present invention is one in which a dengue virus of any one or more of serotypes 1, 3 or 4 is/are the dominant circulating serotype(s), i.e. at least 50%, at least 60%, at least 70%, at least 80%, at least 90%, at least 95% or 100% of the cases of dengue disease are caused by dengue virus of serotypes 1, 3 or 4.

The term “RNA equivalent” of a given DNA sequence, as used herein, refers to a sequence wherein deoxythymidines have been replaced by uridines. Since the DNA sequences in question constitute the cDNA sequences of the dengue viruses, the equivalent RNA sequences constitute the positive strand RNA of those dengue viruses.

### Overview of Several Embodiments

The present inventors have, for the first time, demonstrated the efficacy of a vaccine composition in protecting a human subject against dengue disease.

The present invention relates to a dengue virus serotype 2 vaccine composition comprising:
(i) a dengue antigen selected from the group consisting of:
   (a) a live attenuated dengue virus;
   (b) an inactivated dengue virus;
   (c) a live attenuated or inactivated chimeric dengue virus;
   (d) a dengue virus-like particle (VLP); and
   (e) a combination of two or more of (a) to (d); or
(ii) a nucleic acid construct or viral vector which is able to express in a human cell a dengue antigen which is a dengue VLP;
   wherein said dengue antigen comprises a polypeptide having at least 90% identity to SEQ ID NO: 12.

In preferred embodiments, said polypeptide has at least 92%, at least 94%, at least 96%, at least 98%, at least 99%, at least 99.5% identity or 100% identity with SEQ ID NO: 12.

Preferably, said dengue antigen is selected from the group consisting of a live attenuated dengue virus and a live attenuated or inactivated chimeric dengue virus. Preferably, said dengue antigen is selected from the group consisting of a live attenuated dengue virus and a live attenuated chimeric dengue virus. Preferably, said dengue antigen is a live attenuated chimeric dengue virus.

Preferably said dengue antigen according to the present invention comprises a polypeptide having at least 90% identity to SEQ ID NO: 12, for example at least 92%, at least 94%, at least 96%, at least 98%, at least 99%, at least 99.5% identity or 100% identity to SEQ ID NO: 12 over the full length of SEQ ID NO: 12.

Preferably, said dengue antigen does not comprise the prM-E sequence of CYD-2, as defined herein.

Preferably, said vaccine composition does not comprise CYD-2.

Preferably, said dengue antigen comprises a polypeptide which comprises a valine residue at the position within the polypeptide that corresponds to position 251 of SEQ ID NO: 12.

Preferably, said dengue antigen comprises a polypeptide which comprises a methionine residue at the position within the polypeptide that corresponds to position 6 of SEQ ID NO: 12.

Preferably, said dengue antigen comprises a polypeptide which comprises a valine residue at the position within the polypeptide that corresponds to position 129 of SEQ ID NO: 12.

Preferably, said dengue antigen comprises a polypeptide which comprises an isoleucine residue at the position within the polypeptide that corresponds to position 129 of SEQ ID NO: 12.

Preferably, said dengue antigen comprises a polypeptide which comprises an isoleucine residue at the position within the polypeptide that corresponds to position 141 of SEQ ID NO: 12.

Preferably, said dengue antigen comprises a polypeptide which comprises an isoleucine residue at the position within the polypeptide that corresponds to position 164 of SEQ ID NO: 12.

Preferably, said dengue antigen comprises a polypeptide which comprises an aspartate residue at the position within the polypeptide that corresponds to position 203 of SEQ ID NO: 12.

Preferably, said dengue antigen comprises a polypeptide which comprises an asparagine residue at the position within the polypeptide that corresponds to position 203 of SEQ ID NO: 12.

Preferably, said dengue antigen comprises a polypeptide which comprises a threonine residue at the position within the polypeptide that corresponds to position 226 of SEQ ID NO: 12.

Preferably, said dengue antigen comprises a polypeptide which comprises a glycine residue at the position within the polypeptide that corresponds to position 228 of SEQ ID NO: 12.

Preferably, said dengue antigen comprises a polypeptide which comprises an isoleucine residue at the position within the polypeptide that corresponds to position 308 of SEQ ID NO: 12.

Preferably, said dengue antigen comprises a polypeptide which comprises a valine residue at the position within the polypeptide that corresponds to position 308 of SEQ ID NO: 12.

Preferably, said dengue antigen comprises a polypeptide which comprises a threonine residue at the position within the polypeptide that corresponds to position 478 of SEQ ID NO: 12.

Preferably, said dengue antigen comprises a polypeptide which comprises a valine residue at the position within the polypeptide that corresponds to position 484 of SEQ ID NO: 12.

Preferably, said dengue antigen comprises a polypeptide which comprises an isoleucine residue at the position within the polypeptide that corresponds to position 484 of SEQ ID NO: 12.

Preferably, said dengue antigen comprises a polypeptide which comprises a isoleucine residue at the position within the polypeptide that corresponds to position 485 of SEQ ID NO: 12.

Preferably, said dengue antigen comprises a polypeptide which comprises a alanine residue at the position within the polypeptide that corresponds to position 491 of SEQ ID NO: 12.

Preferably, said dengue antigen comprises a polypeptide having at least 90% sequence identity to SEQ ID NO: 3.

In preferred embodiments, said polypeptide has at least 92%, at least 94%, at least 96%, at least 98%, at least 99%, at least 99.5% identity or 100% identity with SEQ ID NO: 3.

Preferably, a composition of the present invention comprises a polypeptide having at least 90%, at least 92%, at least 94%, at least 96%, at least 98%, at least 99%, at least 99.5% identity or 100% identity with SEQ ID NO: 3. Preferably a dengue antigen according to the present invention comprises a polypeptide having at least 90% identity to SEQ ID NO: 12, for example at least 92%, at least 94%, at least 96%, at least 98%, at least 99%, at least 99.5% identity or 100% identity to SEQ ID NO: 12 over the full length of SEQ ID NO: 12. Preferably, said dengue antigen comprises a polypeptide which comprises a glycine residue at the position within the polypeptide that corresponds to position 15 of SEQ ID NO: 3.

Preferably, said dengue antigen comprises a polypeptide which comprises a serine residue at the position within the polypeptide that corresponds to position 15 of SEQ ID NO: 3.

Preferably, said dengue antigen comprises a polypeptide which comprises a leucine residue at the position within the polypeptide that corresponds to position 24 of SEQ ID NO: 3.

Preferably, said dengue antigen comprises a polypeptide which comprises an isoleucine residue at the position within the polypeptide that corresponds to position 39 of SEQ ID NO: 3.

Preferably, said dengue antigen comprises a polypeptide which comprises a methionine residue at the position within the polypeptide that corresponds to position 39 of SEQ ID NO: 3.

Preferably, said dengue antigen comprises a polypeptide which comprises a valine residue at the position within the polypeptide that corresponds to position 120 of SEQ ID NO: 3.

Preferably, said dengue antigen comprises a polypeptide which comprises an alanine residue at the position within the polypeptide that corresponds to position 120 of SEQ ID NO: 3.

Preferably, said dengue antigen comprises a polypeptide which comprises a threonine residue at the position within the polypeptide that corresponds to position 125 of SEQ ID NO: 3.

Preferably, the polypeptides as defined herein (as comprised within the dengue antigens as comprised within the vaccine compositions of the present invention) comprise a threonine residue at the position within the polypeptide that corresponds to position 125 of SEQ ID NO: 3 and a valine residue at the position within the polypeptide that corresponds to position 417 of SEQ ID NO: 3.

Preferably, the polypeptides which are encoded by the nucleotide sequences as defined herein (i.e as comprised within the vaccine compositions of the present invention) comprise a leucine residue at the position within the polypeptide that corresponds to position 24 of SEQ ID NO: 3, a threonine residue at the position within the polypeptide that corresponds to position 125 of SEQ ID NO: 3 and a valine residue at the position within the polypeptide that corresponds to position 417 of SEQ ID NO: 3.

Preferably, a polypeptide (as comprised within a dengue antigen as comprised within a vaccine composition of the invention) comprises (i) the sequence as set forth in SEQ ID NO: 13 or a sequence having at least 1 and no more than 5 amino acid substitutions with respect to the sequence as set forth in SEQ ID NO: 13; (ii) the sequence as set forth in SEQ ID NO: 14 or a sequence having at least 1 and no more than 5 amino acid substitutions with respect to the sequence as set forth in SEQ ID NO: 14; (iii) the sequence as set forth in SEQ ID NO: 15 or a sequence having at least 1 and no more than 5 amino acid substitutions with respect to the sequence as set forth in SEQ ID NO: 15; (iv) the sequence as set forth in SEQ ID NO: 16 or a sequence having at least 1 and no more than 5 amino acid substitutions with respect to the sequence as set forth in SEQ ID NO: 16; (v) the sequence as set forth in SEQ ID NO: 18 or a sequence having at least 1 and no more than 5 amino acid substitutions with respect to the sequence as set forth in SEQ ID NO: 18; or (vi) the sequence as set forth in SEQ ID NO: 26 or a sequence having at least 1 and no more than 5 amino acid substitutions with respect to the sequence as set forth in SEQ ID NO: 26. Preferably when said sequences comprise an amino acid substitution, said sequences have at least 1 and no more than 4 amino acid substitutions, preferably at least 1 and no more than 3 amino acid substitutions, preferably 1 or 2 amino acid substitutions, preferably 1 amino acid substitution. Preferably at most two, preferably one preferably none of the substitutions are high impact amino acid substitutions (i.e. achieving a score of >25 in the impact scoring method disclosed in Example 2); preferably at most three, preferably two, preferably one, preferably none of the substitutions are median impact amino acid substitutions (i.e. achieving a score of >10 to 25 in the impact scoring method disclosed in Example 2); preferably at most five, preferably four, preferably three, preferably two, preferably one, preferably none of the substitutions are low impact amino acid substitutions (i.e. achieving a score of >0 to 10 in the impact scoring method disclosed in Example 2); preferably all said substitutions are no impact amino acid substitutions (i.e. achieving a score of 0 in the impact scoring method disclosed in Example 2). Preferably said substitutions do not occur at the positions within said sequences corresponding to positions 226, 228 and 251 of SEQ ID NO: 12. Preferably a dengue antigen comprising said polypeptide leads to a balanced immune response when used in the context of a tetravalent composition. Preferably when a vaccine composition comprising a dengue antigen comprising said polypeptide further comprises a dengue antigen of serotypes 1, 3 and 4 as defined herein, said vaccine composition produces a balanced immune response when administered to a mammal, preferably a human.

Preferably said dengue antigen comprises a polypeptide comprising a sequence selected from the group consisting of SEQ ID NO: 13; SEQ ID NO: 14, SEQ ID NO: 15 SEQ ID NO: 16; SEQ ID NO: 18 and SEQ ID NO: 26.

Preferably said dengue antigen comprises a polypeptide comprising a sequence selected from the group consisting of SEQ ID NO: 13 and SEQ ID NO: 16.

Preferably a dengue antigen (as comprised within a vaccine composition of the invention) comprises a polypeptide comprising: (i) the sequence as set forth in SEQ ID NO: 19 or a sequence having at least 1 and no more than 5 amino acid substitutions with respect to the sequence as set forth in SEQ ID NO: 19; (ii) the sequence as set forth in SEQ ID NO: 20 or a sequence having at least 1 and no more than 5 amino acid substitutions with respect to the sequence as set forth in SEQ ID NO: 20; (iii) the sequence as set forth in SEQ ID NO: 21 or a sequence having at least 1 and no more than 5 amino acid substitutions with respect to the sequence as set forth in SEQ ID NO: 21; (iv) the sequence as set forth in SEQ ID NO: 22 or a sequence having at least 1 and no more than 5 amino acid substitutions with respect to the sequence as set forth in SEQ ID NO: 22; (v) the sequence as set forth in SEQ ID NO: 23 or a sequence having at least 1 and no more than 5 amino acid substitutions with respect to the sequence as set forth in SEQ ID NO: 23 or (vi) the sequence as set forth in SEQ ID NO: 27 or a sequence having at least 1 and no more than 5 amino acid substitutions with respect to the sequence as set forth in SEQ ID NO: 27. Preferably when said sequences comprise an amino acid substitution, said sequences have at least 1 and no more than 4 amino acid substitutions, preferably at least 1 and no more than 3 amino acid substitutions, preferably 1 or 2 amino acid substitutions, preferably 1 amino acid substitution. Preferably at most two, preferably one preferably none of the substitutions are high impact amino acid substitutions (i.e. achieving a score of >25 in the impact scoring method disclosed in Example 2); preferably at most three, preferably two, preferably one, preferably none of the substitutions are median impact amino acid substitutions (i.e. achieving a score of >10 to 25 in the impact scoring method disclosed in Example 2); preferably at most five, preferably four, preferably three, preferably two, preferably one, preferably none of the substitutions are low impact amino acid substitutions (i.e. achieving a score of >0 to 10 in the impact scoring method disclosed in Example 2); preferably all said substitutions are no impact amino acid substitutions (i.e. achieving a score of 0 in the impact scoring method disclosed in Example 2). Preferably said substitutions do not occur at the position within said sequences corresponding to position 125 of SEQ ID NO: 3. Preferably a dengue antigen comprising said polypeptide leads to a balanced immune response when used in the context of a tetravalent composition. Preferably when a vaccine composition comprising a dengue antigen comprising said polypeptide further comprises a dengue antigen of serotypes 1, 3 and 4 as defined herein, said vaccine composition produces a balanced immune response when administered to a mammal, preferably a human.

Preferably a dengue antigen (as comprised within a vaccine composition of the invention) comprises a polypeptide comprising a sequence selected from the group consisting of SEQ ID NO: 19; SEQ ID NO: 20, SEQ ID NO: 21; SEQ ID NO: 22; SEQ ID NO: 23 and SEQ ID NO: 27.

Preferably a dengue antigen (as comprised within a vaccine composition of the invention) comprises:
i) a polypeptide having the sequence as set forth in SEQ ID NO: 13 or a polypeptide having at least 1 and no more than 5 amino acid substitutions with respect to the sequence as set forth in SEQ ID NO: 13; and
   a polypeptide having the sequence as set forth in SEQ ID NO: 19 or a polypeptide having at least 1 and no more than 5 amino acid substitutions with respect to the sequence as set forth in SEQ ID NO: 19;
ii) a polypeptide having the sequence as set forth in SEQ ID NO: 14 or a polypeptide having at least 1 and no more than 5 amino acid substitutions with respect to the sequence as set forth in SEQ ID NO: 14; and
   a polypeptide having the sequence as set forth in SEQ ID NO: 20 or a polypeptide having at least 1 and no more than 5 amino acid substitutions with respect to the sequence as set forth in SEQ ID NO: 20;
iii) a polypeptide having the sequence as set forth in SEQ ID NO: 15 or a polypeptide having at least 1 and no more than 5 amino acid substitutions with respect to the sequence as set forth in SEQ ID NO: 15; and
   a polypeptide having the sequence as set forth in SEQ ID NO: 21 or a polypeptide having at least 1 and no more than 5 amino acid substitutions with respect to the sequence as set forth in SEQ ID NO: 21;
iv) a polypeptide having the sequence as set forth in SEQ ID NO: 16 or a polypeptide having at least 1 and no more than 5 amino acid substitutions with respect to the sequence as set forth in SEQ ID NO: 16; and
   a polypeptide having the sequence as set forth in SEQ ID NO: 22 or a polypeptide having at least 1 and no more than 5 amino acid substitutions with respect to the sequence as set forth in SEQ ID NO: 22;
v) a polypeptide having the sequence as set forth in SEQ ID NO: 18 or a polypeptide having at least 1 and no more than 5 amino acid substitutions with respect to the sequence as set forth in SEQ ID NO: 18; and
   a polypeptide having the sequence as set forth in SEQ ID NO: 23 or a polypeptide having at least 1 and no more than 5 amino acid substitutions with respect to the sequence as set forth in SEQ ID NO: 23; or
vi) a polypeptide having the sequence as set forth in SEQ ID NO: 26 or a polypeptide having at least 1 and no more than 5 amino acid substitutions with respect to the sequence as set forth in SEQ ID NO: 26; and
   a polypeptide having the sequence as set forth in SEQ ID NO: 27 or a polypeptide having at least 1 and no more than 5 amino acid substitutions with respect to the sequence as set forth in SEQ ID NO: 27.

Preferably when said sequences comprise an amino acid substitution, said sequences have at least 1 and no more than 4 amino acid substitutions, preferably at least 1 and no more than 3 amino acid substitutions, preferably 1 or 2 amino acid substitutions, preferably 1 amino acid substitution. Preferably at most two, preferably one preferably none of the substitutions are high impact amino acid substitutions (i.e. achieving a score of >25 in the impact scoring method disclosed in Example 2); preferably at most three, preferably two, preferably one, preferably none of the substitutions are median impact amino acid substitutions (i.e. achieving a score of >10 to 25 in the impact scoring method disclosed in Example 2); preferably at most five, preferably four, preferably three, preferably two, preferably one, preferably none of the substitutions are low impact amino acid substitutions (i.e. achieving a score of >0 to 10 in the impact scoring method disclosed in Example 2); preferably all said substitutions are no impact amino acid substitutions (i.e. achieving a score of 0 in the impact scoring method disclosed in Example 2). Preferably said substitutions do not occur at the positions within said sequences corresponding to positions 226, 228 and 251 of SEQ ID NO: 12 and the position corresponding to position 125 of SEQ ID NO: 3. Preferably a dengue antigen comprising said polypeptides leads to a balanced immune response when used in the context of a tetravalent composition. Preferably when a vaccine composition comprising a dengue antigen comprising said polypeptides further comprises a dengue antigen of serotypes 1, 3 and 4 as defined herein, said vaccine composition produces a balanced immune response when administered to a mammal, preferably a human.

Preferably a dengue antigen (as comprised within a vaccine composition of the invention) comprises: i) a polypeptide of SEQ ID NO: 13 and a polypeptide of SEQ ID NO: 19; ii) a polypeptide of SEQ ID NO: 14 and a polypeptide of SEQ ID NO: 20; iii) a polypeptide of SEQ ID NO: 15 and a polypeptide of SEQ ID NO: 21; iv) a polypeptide of SEQ ID NO: 16 and a polypeptide of SEQ ID NO: 22; v) a polypeptide of SEQ ID NO: 18 and a polypeptide of SEQ ID NO: 23 or vi) a polypeptide of SEQ ID NO: 26 and a polypeptide of SEQ ID NO: 27.

Preferably said dengue antigen comprises a polypeptide comprising a sequence selected from the group consisting of SEQ ID NO: 8; SEQ ID NO: 9, SEQ ID NO: 10 and SEQ ID NO: 11.

Preferably said dengue antigen comprises a polypeptide comprising a sequence selected from the group consisting of SEQ ID NO: 8 and SEQ ID NO: 11.

Preferably a composition of the present invention comprises a dengue antigen selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence encoding a polypeptide comprising a polypeptide as defined herein.

The present invention is also directed to a vaccine composition comprising a dengue antigen of serotype 2 selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence having at least 90% sequence identity to a sequence selected from the group consisting of the RNA equivalent of SEQ ID NO: 1, the RNA equivalent of SEQ ID NO: 4, the RNA equivalent of SEQ ID NO: 5, the RNA equivalent of SEQ ID NO: 6, the RNA equivalent of SEQ ID NO: 7 and SEQ ID NO: 25. References to at least 90% sequence identity to the RNA equivalent of SEQ ID NO: 1, the RNA equivalent of SEQ ID NO: 4, the RNA equivalent of SEQ ID NO: 5, the RNA equivalent of SEQ ID NO: 6, the RNA equivalent of SEQ ID NO: 7 or SEQ ID NO: 25 may preferably be read herein as at least 92%, at least 93%, at least 94%, at least 95%, at least 96%, at least 97%, at least 98%, at least 99%, at least 99.5% or 100% sequence identity. When nucleotide sequences of this embodiment of the invention encode polypeptides comprising one or more amino acid substitutions with respect to the polypeptides encoded by SEQ ID NO: 1, SEQ ID NO: 4, SEQ ID NO: 5, SEQ ID NO: 6, SEQ ID NO: 7 and SEQ ID NO: 25, preferably at most two, preferably one preferably none of the substitutions are high impact amino acid substitutions (i.e. achieving a score of >25 in the impact scoring method disclosed in Example 2); preferably at most three, preferably two, preferably one, preferably none of the substitutions are median impact amino acid substitutions (i.e. achieving a score of >10 to 25 in the impact scoring method disclosed in Example 2); preferably at most five, preferably four, preferably three, preferably two, preferably one, preferably none of the substitutions are low impact amino acid substitutions (i.e. achieving a score of >0 to 10 in the impact scoring method disclosed in Example 2); preferably all said substitutions are no impact amino acid substitutions (i.e. achieving a score of 0 in the impact scoring method disclosed in Example 2). Preferably said substitutions do not occur at the positions within said polypeptides corresponding to positions 226, 228 and 251 of SEQ ID NO: 12 and the positions within said polypeptides corresponding to positions 24 and 125 of SEQ ID NO: 3. Preferably the vaccine compositions comprising a dengue antigen of serotype 2 of this embodiment of the invention lead to a balanced immune response when used in the context of a tetravalent composition. Preferably a dengue antigen of serotype 2 according to this embodiment of the invention further comprises a dengue antigen of serotype 1, a dengue antigen of serotype 3 and a dengue antigen of serotype 4 as described elsewhere herein. Preferably when a vaccine composition comprising a dengue antigen of serotype 2 according to this embodiment of the invention further comprises a dengue antigen of serotypes 1, 3 and 4 as defined herein, said vaccine composition produces a balanced immune response when administered to a mammal, preferably a human. When a vaccine composition of the present invention comprises a dengue antigen of serotype 2 which comprises a nucleotide sequence having at least 90% identity to the RNA equivalent of SEQ ID NO: 7, said vaccine composition is preferably either: (i) a vaccine composition comprising a dengue antigen comprising a nucleotide sequence having at least 90% identity to the RNA equivalent of SEQ ID NO: 7, wherein said dengue antigen is not CYD-MD or (ii) a vaccine composition comprising a dengue antigen which is CYD-MD. Said vaccine composition is also preferably either: (i) a vaccine composition comprising a dengue antigen comprising a nucleotide sequence having at least 90% identity to the RNA equivalent of SEQ ID NO: 7, wherein said vaccine composition does not comprise prM-E sequences from MD1280 or (ii) a vaccine composition comprising prM-E sequences from MD1280. Said vaccine composition is also preferably either: (i) a vaccine composition comprising a dengue antigen comprising a nucleotide sequence having at least 90% identity to the RNA equivalent of SEQ ID NO: 7, wherein said vaccine composition does not comprise a dengue antigen comprising the M and E sequences of CYD-MD or (ii) a vaccine composition comprising a dengue antigen comprising the M and E sequences of CYD-MD. Said vaccine composition is also preferably either (i) a vaccine composition comprising a dengue antigen comprising a nucleotide sequence having at least 90% identity to the RNA equivalent of SEQ ID NO: 7, wherein said vaccine composition does not comprise a chimeric dengue virus of serotype 2 generated using the prM-E sequences of MD1280 (SEQ ID NO: 11) or (ii) a vaccine composition comprising a chimeric dengue virus of serotype 2 generated using the prM-E sequences of MD1280 (SEQ ID NO: 11). Said vaccine composition is also preferably either (i) a vaccine composition comprising a dengue antigen comprising a nucleotide sequence having at least 90% identity to the RNA equivalent of SEQ ID NO: 7, wherein said vaccine composition does not comprise a dengue antigen comprising the prM and E sequences of CYD-MD or (ii) a vaccine composition comprising a dengue antigen comprising the prM and E sequences of CYD-MD. Said vaccine composition is also preferably either (i) a vaccine composition comprising a dengue antigen comprising a nucleotide sequence having at least 90% identity to the RNA equivalent of SEQ ID NO: 7, wherein said vaccine composition does not comprise a dengue antigen comprising a polypeptide of SEQ ID NO: 16 and a polypeptide of SEQ ID NO: 22 (or a dengue antigen comprising a nucleotide sequence encoding a protein comprising said polypeptides) or (ii) a vaccine composition comprising a dengue antigen comprising a polypeptide of SEQ ID NO: 16 and a polypeptide of SEQ ID NO: 22 (or a dengue antigen comprising a nucleotide sequence encoding a protein comprising said polypeptides). Preferably, a vaccine composition of the present invention which comprises a dengue antigen of serotype 2 which comprises a nucleotide sequence having at least 90% identity to the RNA equivalent of SEQ ID NO: 7 does not comprise: (i) a chimeric virus comprising the prM and E amino acid sequences of MD1280 or (ii) a dengue antigen of serotype 2 which comprises the prM-E sequence of CYD-MD (SEQ ID NO: 11). When a vaccine composition of the present invention comprises a dengue antigen of serotype 2 which comprises a nucleotide sequence having at least 90% identity to the RNA equivalent of SEQ ID NO: 4, said vaccine composition is preferably either: (i) a vaccine composition comprising a dengue antigen comprising a nucleotide sequence having at least 90% identity to the RNA equivalent of SEQ ID NO: 4, wherein said dengue antigen is not CYD-LAV or (ii) a vaccine composition comprising a dengue antigen which is CYD-LAV. Said vaccine composition is also preferably either: (i) a vaccine composition comprising a dengue antigen comprising a nucleotide sequence having at least 90% identity to the RNA equivalent of SEQ ID NO: 4, wherein said vaccine composition does not comprise prM-E sequences from LAV2 or (ii) a vaccine composition comprising prM-E sequences from LAV2. Said vaccine composition is also preferably either: (i) a vaccine composition comprising a dengue antigen comprising a nucleotide sequence having at least 90% identity to the RNA equivalent of SEQ ID NO: 4, wherein said vaccine composition does not comprise a dengue antigen comprising the M and E sequences of CYD-LAV or (ii) a vaccine composition comprising a dengue antigen comprising the M and E sequences of CYD-LAV. Said vaccine composition is also preferably either (i) a vaccine composition comprising a dengue antigen comprising a nucleotide sequence having at least 90% identity to the RNA equivalent of SEQ ID NO: 4, wherein said vaccine composition does not comprise a chimeric dengue virus of serotype 2 generated using the prM-E sequences of LAV2 (SEQ ID NO: 8) or (ii) a vaccine composition comprising a chimeric dengue virus of serotype 2 generated using the prM-E sequences of LAV2 (SEQ ID NO: 8). Said vaccine composition is also preferably either (i) a vaccine composition comprising a dengue antigen comprising a nucleotide sequence having at least 90% identity to the RNA equivalent of SEQ ID NO: 4, wherein said vaccine composition does not comprise a dengue antigen comprising the prM and E sequences of CYD-LAV or (ii) a vaccine composition comprising a dengue antigen comprising the prM and E sequences of CYD-LAV. Said vaccine composition is also preferably either (i) a vaccine composition comprising a dengue antigen comprising a nucleotide sequence having at least 90% identity to the RNA equivalent of SEQ ID NO: 4, wherein said vaccine composition does not comprise a dengue antigen comprising a polypeptide of SEQ ID NO: 13 and a polypeptide of SEQ ID NO: 19 (or a dengue antigen comprising a nucleotide sequence encoding a protein comprising said polypeptides) or (ii) a vaccine composition comprising a dengue antigen comprising a polypeptide of SEQ ID NO: 13 and a polypeptide of SEQ ID NO: 19 (or a dengue antigen comprising a nucleotide sequence encoding a protein comprising said polypeptides). Preferably, a vaccine composition of the present invention which comprises a dengue antigen of serotype 2 which comprises a nucleotide sequence having at least 90% identity to the RNA equivalent of SEQ ID NO: 4 does not comprise: (i) a chimeric virus comprising the prM and E amino acid sequences of LAV2 or (ii) a dengue antigen of serotype 2 which comprises the prM-E sequence of CYD-LAV (SEQ ID NO: 8).

The present invention is also directed to a vaccine composition comprising a dengue antigen of serotype 2 selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence having at least 1 and no more than 20 nucleotide substitutions with respect to a sequence selected from the group consisting of the RNA equivalent of SEQ ID NO: 1, the RNA equivalent of SEQ ID NO: 4, the RNA equivalent of SEQ ID NO: 5, the RNA equivalent of SEQ ID NO: 6, the RNA equivalent of SEQ ID NO: 7 and SEQ ID NO: 25. When nucleotide sequences of this embodiment of the invention encode polypeptides comprising one or more amino acid substitutions with respect to the polypeptides encoded by SEQ ID NO: 1, SEQ ID NO: 4, SEQ ID NO: 5, SEQ ID NO: 6, SEQ ID NO: 7 and SEQ ID NO: 25, preferably at most two, preferably one preferably none of the substitutions are high impact amino acid substitutions (i.e. achieving a score of >25 in the impact scoring method disclosed in Example 2); preferably at most three, preferably two, preferably one, preferably none of the substitutions are median impact amino acid substitutions (i.e. achieving a score of >10 to 25 in the impact scoring method disclosed in Example 2); preferably at most five, preferably four, preferably three, preferably two, preferably one, preferably none of the substitutions are low impact amino acid substitutions (i.e. achieving a score of >0 to 10 in the impact scoring method disclosed in Example 2); preferably all said substitutions are no impact amino acid substitutions (i.e. achieving a score of 0 in the impact scoring method disclosed in Example 2). Preferably said substitutions do not occur at the positions within said polypeptides corresponding to positions 226, 228 and 251 of SEQ ID NO: 12 and the positions within said polypeptides corresponding to positions 24 and 125 of SEQ ID NO: 3. Preferably the vaccine compositions comprising a dengue antigen of serotype 2 of this embodiment of the invention lead to a balanced immune response when used in the context of a tetravalent composition. Preferably a dengue antigen of serotype 2 according to this embodiment of the invention further comprises a dengue antigen of serotype 1, a dengue antigen of serotype 3 and a dengue antigen of serotype 4 as described elsewhere herein. Preferably when a vaccine composition comprising a dengue antigen of serotype 2 according to this embodiment of the invention further comprises a dengue antigen of serotypes 1, 3 and 4 as defined herein, said vaccine composition produces a balanced immune response when administered to a mammal, preferably a human.

When a vaccine composition of the present invention comprises a dengue antigen of serotype 2 as defined herein which comprises a polypeptide having at least 90%, at least 92%, at least 94%, at least 96%, at least 98%, at least 99%, at least 99.5% or 100% identity to SEQ ID NO: 12, wherein said polypeptide comprises an isoleucine residue at the position within the polypeptide that corresponds to position 485 of SEQ ID NO: 12 or an alanine residue at the position within the polypeptide that corresponds to position 491 of SEQ ID NO: 12, said vaccine composition preferably (i) is not CYD-MD; (ii) does not comprise the prM-E sequence from MD1280; (iii) does not comprise a chimeric dengue virus of serotype 2 generated using the prM-E sequences of MD1280 (SEQ ID NO: 11); (iv) does not comprise a dengue antigen comprising the prM and E sequences of CYD-MD; (v) does not comprise a dengue antigen comprising a polypeptide of SEQ ID NO: 16 and a polypeptide of SEQ ID NO: 22 (or a dengue antigen comprising a nucleotide sequence encoding a protein comprising said polypeptides); (vi) does not comprise a chimeric virus comprising the prM and E amino acid sequences of MD1280; (vii) does not comprise a dengue antigen of serotype 2 which comprises the prM-E sequence of CYD-MD (SEQ ID NO: 11) and/or (viii) does not comprise a dengue antigen of serotype 2 which comprises the M and E sequences of CYD-MD. When a vaccine composition of the present invention comprises a dengue antigen of serotype 2 as defined herein which comprises a polypeptide having at least 90%, at least 92%, at least 94%, at least 96%, at least 98%, at least 99%, at least 99.5% or 100% identity to SEQ ID NO: 12, wherein said polypeptide comprises a methionine residue at the position within the polypeptide that corresponds to position 6 of SEQ ID NO: 12 or a threonine residue at the position within the polypeptide that corresponds to position 478 of SEQ ID NO: 12, said vaccine composition preferably (i) is not CYD-LAV; (ii) does not comprise the prM-E sequence from LAV2; (iii) does not comprise a chimeric dengue virus of serotype 2 generated using the prM-E sequences of LAV2 (SEQ ID NO: 8); (iv) does not comprise a dengue antigen comprising the prM and E sequences of CYD-LAV; (v) does not comprise a dengue antigen comprising a polypeptide of SEQ ID NO: 13 and a polypeptide of SEQ ID NO: 19 (or a dengue antigen comprising a nucleotide sequence encoding a protein comprising said polypeptides); (vi) does not comprise a chimeric virus comprising the prM and E amino acid sequences of LAV2; (vii) does not comprise a dengue antigen of serotype 2 which comprises the prM-E sequence of CYD-LAV (SEQ ID NO: 8) and/or (viii) does not comprise a dengue antigen of serotype 2 which comprises the M and E sequences of CYD-LAV.

Preferably said dengue antigen comprises a polypeptide which comprises no more than 1, no more than 2, no more than 3, no more than 4, no more than 5, nor more than 6, no more than 7, no more than 8, no more than 9, no more than 10, no more than 11 or no more than 12 minor amino acid residues, wherein a minor amino acid residue at a given position of a prM-E or E sequence is defined as an amino acid that appears in less than 15% of dengue virus prM-E or E sequences of serotype 2 at that position.

Preferably the dengue disease according to the present invention is virologically-confirmed dengue disease.

Preferably a human subject according to the present invention is less than 18 years of age or less than 12 years of age. For example, a human subject according to the present invention may be 0-17 years of age, 0-11 years of age, 4-17 years of age, 4-11 years of age, 4-6 years of age, 6-8 years of age, 8-10 years of age, 2-8 years of age, 2-11 years of age, 2-14 years of age, 9-16 years of age, 12-17 years of age or 18-45 years of age. More preferably, a human subject according to the present invention is 4-11 years of age, 2-14 years of age or 9-16 years of age. A human subject according to the present invention may be at least 9 months old or less than 9 months old. For instance a human subject according to the present invention may be 9 months to 16 years of age, 9 months to 14 years of age, 9 months to 11 years of age or 9 months to 8 years of age. A human subject according to the present invention may be at least 9 months old, with no history of severe allergy to any component of the vaccine composition as defined herein, no congenital or acquired immune deficiency, no symptomatic HIV infection and said subject should not be pregnant or breast feeding.

A human subject to which a vaccine composition of the present invention is to be administered is preferably a person at risk of infection, for instance a person travelling in an area where dengue fever is present, i.e. a dengue endemic area, or a resident of such an area. Preferably a human subject of the present invention resides in a dengue endemic area. Dengue endemic areas according to the present invention include most of the tropics and sub-tropics, for instance any country identified as an endemic country by the WHO. For instance, a dengue endemic area according to the present invention may comprise those American countries or parts thereof which fall within the tropics and sub-tropics. A dengue endemic area according to the present invention may thus comprise any one or more of the following: Brazil, Venezuela, Colombia, Ecuador, Peru, Bolivia, Paraguay, Panama, Costa Rica, Nicaragua, Honduras, El Salvador, Guatemala, Belize, Mexico, the USA and the islands of the Caribbean. In a particular embodiment, a dengue endemic area of the present invention may consist of the following: Brazil, Colombia, Honduras, Mexico and Puerto Rico. A dengue endemic area according to the present invention may also include south Asian and Oceania countries within the tropics and sub-tropics. A dengue endemic area according to the present invention may thus consist of any one or more of the following: India, Myanmar (Burma), Thailand, Laos, Vietnam, Cambodia, Indonesia, Malaysia, Singapore, the Philippines, Taiwan, Papua New Guinea and Australia. In a dengue endemic area according to the present invention, a particular serotype, strain or genotype of wild type dengue virus may be the dominant circulating strain. For example, a dengue virus of serotype 2 may be characterised as having an Asian I or an Asian/American genotype. Asian/American genotype strains are characterised by at least one of, at least two of, at least three of, at least four of, at least five of or all six of the following residues Arg, Asn, Asp, Thr, Gly and His at positions prM-16, E-83, E-203, E-226, E-228 and E-346 respectively (wherein prM-16 designates position 16 of the prM protein and E-83 etc. designates position 83 of the E protein). Asian I genotype strains are characterised by at least one of, at least two of, at least three of, at least four of, at least five of or all six of the following residues Ile, Lys, Asn, Arg, Glu and Tyr at positions prM-16, E-83, E-203, E-226, E-228 and E-346 respectively (see Table 1 of Hang et al., PLoS NTD, 4(7): e757). A preferred dengue endemic area according to the present invention is one in which a dengue virus having an Asian/American genotype is the dominant circulating strain, i.e. at least 50%, at least 60%, at least 70%, at least 80%, at least 90%, at least 95% or 100% of the cases of dengue disease in said dengue endemic area are caused by dengue virus having an Asian/American genotype. A preferred dengue endemic area according to the present invention is one in which a dengue virus of any one or more of serotypes 1, 3 or 4 is/are the dominant circulating serotype(s), i.e. at least 50%, at least 60%, at least 70%, at least 80%, at least 90%, at least 95% or 100% of the cases of dengue disease are caused by dengue virus of serotypes 1, 3 or 4.

A vaccine composition of the present invention may be administered to a flavivirus immune subject, for example a dengue-immune subject, or a vaccine composition of the present invention may be administered to a flavivirus-naïve subject. Advantageously, a vaccine composition of the present invention is administered to a flavivirus-immune subject, for example a dengue-immune subject.

Preferably, a composition according to the present invention, e.g. a composition for use in a method according to the present invention, reduces the likelihood or severity of DHF. A reduction in the likelihood of DHF (i.e. a reduction in the probability of contracting DHF) may be measured by comparing the number of cases of DHF in a group of subjects who have received a vaccine composition according to the present invention and the number of cases of DHF in a control group of subjects who have not received a vaccine composition according to the present invention. A reduction in the severity of DHF may be determined by calculating the number of subjects displaying DHF of each of Grades I, II, III or IV in a group of subjects who have received a vaccine composition according to the present invention and comparing those numbers to the equivalent numbers from a control group of subjects who have not received a vaccine composition according to the present invention. For instance, a composition for use in a method according to the present invention preferably reduces the number of cases of Grade I DHF, the number of cases of Grade II DHF, the number of cases of Grade III DHF and/or the number of cases of Grade IV DHF in those subjects receiving the vaccine, when compared to the equivalent number of cases Grade I DHF, Grade II DHF, Grade III DHF and Grade IV DHF occurring in a control group of subjects who have not received a vaccine composition according to the present invention.

Preferably, a composition according to the present invention, e.g. a composition for use in a method according to the present invention, reduces the incidence or likelihood of symptomatic virologically-confirmed dengue disease. Advantageously, a composition according to the present invention, e.g. a composition for use in a method according to the present invention, reduces the incidence or likelihood of symptomatic virologically-confirmed dengue disease caused by dengue virus of serotypes 1, 3 or 4. Advantageously, a composition according to the present invention, e.g. a composition for use in a method according to the present invention, reduces the incidence or likelihood of symptomatic virologically-confirmed dengue disease caused by dengue virus of serotypes 1, 2, 3 or 4. Preferably, a composition according to the present invention, e.g. a composition for use in a method according to the present invention, reduces the rate of hospitalization due to virologically-confirmed dengue disease, i.e. reduces the incidence of hospitalized virologically-confirmed dengue disease. For instance, a composition according to the present invention, e.g. a composition for use in a method according to the present invention, reduces the rate of hospitalization due to virologically-confirmed dengue disease caused by dengue virus of serotypes 1, 3 or 4, i.e. reduces the incidence of hospitalized virologically-confirmed dengue disease caused by dengue virus of serotypes 1, 3 or 4.

Preferably, a composition according to the present invention, e.g. a composition for use in a method according to the present invention, reduces the incidence or likelihood of dengue disease. Advantageously, a composition according to the present invention, e.g. a composition for use in a method according to the present invention, reduces the incidence or likelihood of dengue disease caused by dengue virus of serotypes 1, 3 or 4. Advantageously, a composition according to the present invention, e.g. a composition for use in a method according to the present invention, reduces the incidence or likelihood of dengue disease caused by dengue virus of serotypes 1, 2, 3 or 4. Preferably, a composition according to the present invention, e.g. a composition for use in a method according to the present invention, reduces the rate of hospitalization due to dengue disease, i.e. reduces the incidence of hospitalized dengue disease. For instance, a composition according to the present invention, e.g. a composition for use in a method according to the present invention, reduces the rate of hospitalization due to dengue disease caused by dengue virus of serotypes 1, 3 or 4, i.e. reduces the incidence of hospitalized dengue disease caused by dengue virus of serotypes 1, 3 or 4.

A vaccine composition according to the present invention may be administered in multiple doses. Doses of a vaccine composition according to the present invention may be administered in an initial vaccination regimen followed by booster vaccinations. For example, a vaccine composition according to the present invention may be administered in one, two or three doses or more than three doses, e.g. four doses. Preferably, the first dose and the third dose are to be administered approximately twelve months apart. For example, an initial vaccination regimen according to the present invention is administered in three doses, wherein the first and third doses of said vaccination regimen are to be administered approximately twelve months apart. Advantageously, a vaccine composition according to the present invention is to be administered in a first dose, a second dose and a third dose. In such an embodiment, said first dose and said third dose may be administered approximately twelve months apart. For instance, a vaccine composition of the present invention may be administered in a first dose, a second dose and a third dose, wherein said second dose is to be administered about six months after said first dose and wherein said third dose is to be administered about twelve months after said first dose. Alternatively, the three doses may be administered at zero months, at about three to four months (e.g. at about three-and-a-half months) and at about twelve months (i.e. a regimen wherein the second dose of the composition is administered at about three-and-a-half months after the first dose, and wherein the third dose of the composition is administered at about twelve months after the first dose).

A vaccine composition according to the present invention may be administered in two doses. Preferably, the first dose and the second dose are to be administered approximately about six to twelve months after the first dose months apart. Preferably, the second dose is to be administered at eight months after the first dose. Preferably the second dose is administered at about eight-and-a-half to nine months after the first dose.

A vaccine composition according to the present invention may be administered in a single dose.

Dengue disease, as defined herein, may be caused by any one of two serotypes of a dengue virus. For example, dengue disease is preferably caused by a dengue virus of serotype 1 or serotype 3, a dengue virus of serotype 1 or serotype 4, a dengue virus of serotype 3 or serotype 4, a dengue virus of serotype 1 or serotype 2, a dengue virus of serotype 2 or serotype 3, a dengue virus of serotype 2 or serotype 4. Dengue disease, as defined herein, is preferably caused by any one of three serotypes of a dengue virus. For example, dengue disease is preferably caused by a dengue virus of serotype 1, 2 or 3, a dengue virus of serotype 1, 3 or 4, a dengue virus of serotype 1, 2 or 4, a dengue virus of serotype 2, 3 or 4. In another embodiment, dengue disease is caused by a dengue virus of serotype 1, a dengue virus of serotype 2, a dengue virus of serotype 3 or a dengue virus of serotype 4.

A vaccine composition according to the present invention, e.g. for use in a method according to the present invention preferably comprises a dengue antigen of serotype 1, a dengue antigen of serotype 2, a dengue antigen of serotype 3 and a dengue antigen of serotype 4. Such a composition may be described herein as a tetravalent composition. For instance, a composition of the present invention, e.g. for use in a method of protecting according to the present invention, may advantageously comprise any one of the following combinations of dengue antigens of serotypes 1, 2, 3 and 4: i) a dengue antigen comprising the prM and E sequences of CYD-1, a dengue antigen comprising the prM and E sequences of CYD-LAV, a chimeric dengue virus comprising the prM and E sequences of CYD-3 and a dengue antigen comprising the prM and E sequences of CYD-4; ii) a dengue antigen comprising the prM and E sequences of CYD-1, a dengue antigen comprising the prM and E sequences of CYD-BID, a dengue antigen comprising the prM and E sequences of CYD-3 and a dengue antigen comprising the prM and E sequences of CYD-4; (iii) a dengue antigen comprising the prM and E sequences of CYD-1, a dengue antigen comprising the prM and E sequences of CYD-PR, a dengue antigen comprising the prM and E sequences of CYD-3 and a dengue antigen comprising the prM and E sequences of CYD-4; (iv) a dengue antigen comprising the prM and E sequences of CYD-1, a dengue antigen comprising the prM and E sequences of CYD-MD, a dengue antigen comprising the prM and E sequences of CYD-3 and a dengue antigen comprising the prM and E sequences of CYD-4;. For instance, a composition of the present invention may also advantageously comprise any one of the following combinations of dengue antigens: i) CYD-1, CYD-LAV, CYD-3 and CYD-4; ii) CYD-1, CYD-BID, CYD-3 and CYD-4; (iii) CYD-1, CYD-PR, CYD-3 and CYD-4 or (iv) CYD-1, CYD-MD, CYD-3 and CYD-4. A composition of the present invention may also advantageously comprise the following combination of dengue antigens: i) a dengue antigen comprising the prM and E sequences of CYD-1, VDV2, a dengue antigen comprising the prM and E sequences of CYD-3 and a dengue antigen comprising the prM and E sequences of CYD-4. For instance, a composition of the present invention may advantageously comprise CYD-1, VDV-2, CYD-3 and CYD-4. A composition of the present invention, as described herein, may advantageously comprise a dengue antigen of serotype 2 which comprises the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. A composition of the present invention, as described herein, may advantageously comprise a dengue antigen of serotype 2 which comprises a sequence having at least 90% identity to the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. For example, said sequence may be at least 91%, at least 92%, at least 93%, at least 94%, at least 95%, at least 96%, at least 97%, at least 98% or at least 99% identical to the prm-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2.

A vaccine composition according to the present invention, e.g. for use in a method according to the present invention, preferably comprises a dengue antigen of serotype 1, a dengue antigen of serotype 2, a dengue antigen of serotype 3 and a dengue antigen of serotype 4. Such a composition may be described herein as a tetravalent composition. For instance, a composition of the present invention, e.g. for use in a method of protecting according to the present invention, may advantageously comprise any one of the following combinations of dengue antigens of serotypes 1, 2, 3 and 4: i) a dengue antigen comprising the M and E sequences of CYD-1, a dengue antigen comprising the M and E sequences of CYD-LAV, a chimeric dengue virus comprising the M and E sequences of CYD-3 and a dengue antigen comprising the M and E sequences of CYD-4; ii) a dengue antigen comprising the M and E sequences of CYD-1, a dengue antigen comprising the M and E sequences of CYD-BID, a dengue antigen comprising the M and E sequences of CYD-3 and a dengue antigen comprising the M and E sequences of CYD-4; (iii) a dengue antigen comprising the M and E sequences of CYD-1, a dengue antigen comprising the M and E sequences of CYD-PR, a dengue antigen comprising the M and E sequences of CYD-3 and a dengue antigen comprising the M and E sequences of CYD-4; (iv) a dengue antigen comprising the M and E sequences of CYD-1, a dengue antigen comprising the M and E sequences of CYD-MD, a dengue antigen comprising the M and E sequences of CYD-3 and a dengue antigen comprising the M and E sequences of CYD-4;. For instance, a composition of the present invention may also advantageously comprise any one of the following combinations of dengue antigens: i) CYD-1, CYD-LAV, CYD-3 and CYD-4; ii) CYD-1, CYD-BID, CYD-3 and CYD-4; (iii) CYD-1, CYD-PR, CYD-3 and CYD-4 or (iv) CYD-1, CYD-MD, CYD-3 and CYD-4. A composition of the present invention may also advantageously comprise the following combination of dengue antigens: i) a dengue antigen comprising the M and E sequences of CYD-1, VDV2, a dengue antigen comprising the M and E sequences of CYD-3 and a dengue antigen comprising the M and E sequences of CYD-4. For instance, a composition of the present invention may advantageously comprise CYD-1, VDV-2, CYD-3 and CYD-4. A composition of the present invention, as described herein, may advantageously comprise a dengue antigen of serotype 2 which comprises the E sequence of CYD-LAV (SEQ ID NO: 13), CYD-BID (SEQ ID NO: 14), CYD-PR (SEQ ID NO: 15) CYD-MD (SEQ ID NO: 16) or SEQ ID NO: 18. In certain embodiments, a composition of the present invention which comprises a dengue antigen comprising the sequence as set forth in SEQ ID NO: 18 is not a vaccine composition of serotype 2 comprising the prM-E sequence from SEQ ID NO: 2. In certain embodiments, a composition of the present invention is either a composition comprising a dengue antigen comprising the sequence as set forth in SEQ ID NO: 18, wherein said composition is not a vaccine composition of the present invention comprising chimeric dengue virus of serotype 2 generated using the prM-E sequence of SEQ ID NO: 2, or it is a composition comprising chimeric dengue virus of serotype 2 generated using the prM-E sequence of SEQ ID NO: 2. A composition of the present invention, as described herein, may advantageously comprise a dengue antigen of serotype 2 which comprises a sequence having at least 90% identity to the E sequence of CYD-LAV (SEQ ID NO: 13), CYD-BID (SEQ ID NO: 14), CYD-PR (SEQ ID NO: 15) CYD-MD (SEQ ID NO: 16) or SEQ ID NO: 18. For example, said sequence may be at least 91%, at least 92%, at least 93%, at least 94%, at least 95%, at least 96%, at least 97%, at least 98% or at least 99% identical to the E sequence of CYD-LAV (SEQ ID NO: 13), CYD-BID (SEQ ID NO: 14), CYD-PR (SEQ ID NO: 15) CYD-MD (SEQ ID NO: 16) or SEQ ID NO: 18.

A composition of the present invention, as described herein, (e.g. a tetravalent formulation, e.g. for use in a method of the present invention), may advantageously comprise a dengue antigen of serotype 2 which comprises a polypeptide selected from the group consisting of SEQ ID NO: 19, SEQ ID NO: 20, SEQ ID NO: 21, SEQ ID NO: 22 or SEQ ID NO: 23. When a vaccine composition of the present invention comprises a dengue antigen of serotype 2 which comprises a polypeptide having the sequence of SEQ ID NO: 19, said vaccine composition is preferably either: (i) a vaccine composition comprising a dengue antigen comprising a polypeptide having the sequence of SEQ ID NO: 19, wherein said vaccine composition does not comprise CYD-LAV or (ii) a vaccine composition comprising CYD-LAV. Said vaccine composition is also preferably either: (i) a vaccine composition comprising a dengue antigen comprising a polypeptide having the sequence of SEQ ID NO: 19, wherein said vaccine composition does not comprise a dengue antigen comprising the M and E sequences of CYD-LAV or (ii) a vaccine composition comprising a dengue antigen comprising the M and E sequences of CYD-LAV. Said vaccine composition is also preferably either: (i) a vaccine composition comprising a dengue antigen comprising a polypeptide having the sequence of SEQ ID NO: 19, wherein said vaccine composition does not comprise a dengue antigen comprising the prM and E sequences of CYD-LAV or (ii) a vaccine composition comprising a dengue antigen comprising the prM and E sequences of CYD-LAV. Said vaccine composition is also preferably either: (i) a vaccine composition comprising a dengue antigen comprising a polypeptide having the sequence of SEQ ID NO: 19, wherein said vaccine composition does not comprise a chimeric dengue virus of serotype 2 generated using the prM-E sequence of LAV-2 or (ii) a vaccine composition comprising a chimeric dengue virus of serotype 2 generated using the prM-E sequence of LAV-2 (SEQ ID NO: 8). Preferably, a vaccine composition of the present invention which comprises a polypeptide having the sequence of SEQ ID NO: 19 does not comprise: (i) a chimeric virus comprising the prM and E amino acid sequences of LAV-2; (ii) a dengue antigen of serotype 2 which comprises the prM-E sequence of CYD-LAV (SEQ ID NO: 8) or (iii) a dengue antigen comprising the prM-E sequence from LAV-2. When a vaccine composition of the present invention comprises a dengue antigen of serotype 2 which comprises a polypeptide having the sequence of SEQ ID NO: 21, said vaccine composition is preferably either: (i) a vaccine composition comprising a dengue antigen comprising a polypeptide having the sequence of SEQ ID NO: 21, wherein said vaccine composition does not comprise CYD-PR or (ii) a vaccine composition comprising CYD-PR. Said vaccine composition is also preferably either: (i) a vaccine composition comprising a dengue antigen comprising a polypeptide having the sequence of SEQ ID NO: 21, wherein said vaccine composition does not comprise a dengue antigen comprising the prM and E sequences of CYD-PR or (ii) a vaccine composition comprising a dengue antigen comprising the prM and E sequences of CYD-PR. Said vaccine composition is also preferably either: (i) a vaccine composition comprising a dengue antigen comprising a polypeptide having the sequence of SEQ ID NO: 21, wherein said vaccine composition does not comprise a chimeric dengue virus of serotype 2 generated using the prM-E sequence of PR/DB023 (SEQ ID NO: 10) or (ii) a vaccine composition comprising a chimeric dengue virus of serotype 2 generated using the prM-E sequence of PR/DB023 (SEQ ID NO: 10). Preferably, a vaccine composition of the present invention which comprises a polypeptide having the sequence of SEQ ID NO: 21 does not comprise: (i) a chimeric virus comprising the prM and E amino acid sequences of PR/DB023; (ii) a dengue antigen of serotype 2 which comprises the prM-E sequence of CYD-PR (SEQ ID NO: 10) or (iii) a dengue antigen comprising the prM-E sequence from PR/DB023. When a vaccine composition of the present invention comprises a dengue antigen of serotype 2 which comprises a polypeptide having the sequence of SEQ ID NO: 22, said vaccine composition is preferably either: (i) a vaccine composition comprising a dengue antigen comprising a polypeptide having the sequence of SEQ ID NO: 22, wherein said vaccine composition does not comprise CYD-MD or (ii) a vaccine composition comprising CYD-MD. Said vaccine composition is also preferably either: (i) a vaccine composition comprising a dengue antigen comprising a polypeptide having the sequence of SEQ ID NO: 22, wherein said vaccine composition does not comprise a dengue antigen comprising the prM and E sequences of CYD-MD or (ii) a vaccine composition comprising a dengue antigen comprising the prM and E sequences of CYD-MD. Said vaccine composition is also preferably either: (i) a vaccine composition comprising a dengue antigen comprising a polypeptide having the sequence of SEQ ID NO: 22, wherein said vaccine composition does not comprise a dengue antigen comprising the M and E sequences of CYD-MD or (ii) a vaccine composition comprising a dengue antigen comprising the M and E sequences of CYD-MD. Said vaccine composition is also preferably either: (i) a vaccine composition comprising a dengue antigen comprising a polypeptide having the sequence of SEQ ID NO: 22, wherein said vaccine composition does not comprise a chimeric dengue virus of serotype 2 generated using the prM-E sequence of MD1280 (SEQ ID NO: 11) or (ii) a vaccine composition comprising a chimeric dengue virus of serotype 2 generated using the prM-E sequence of MD1280 (SEQ ID NO: 11). Preferably, a vaccine composition of the present invention which comprises a polypeptide having the sequence of SEQ ID NO: 22 does not comprise: (i) a chimeric virus comprising the prM and E amino acid sequences of MD1280; (ii) a dengue antigen of serotype 2 which comprises the prM-E sequence of CYD-MD (SEQ ID NO: 11) or (iii) a dengue antigen comprising the prM-E sequence from MD1280. When a vaccine composition of the present invention comprises a dengue antigen of serotype 2 which comprises a polypeptide having the sequence of SEQ ID NO: 23, said vaccine composition is preferably either: (i) a vaccine composition comprising a dengue antigen comprising a polypeptide having the sequence of SEQ ID NO: 23, wherein said vaccine composition does not comprise a chimeric dengue virus of serotype 2 generated using the prM-E sequence SEQ ID NO: 2 or (ii) a vaccine composition comprising a chimeric dengue virus of serotype 2 generated using the prM-E sequence of SEQ ID NO: 2. Preferably, a vaccine composition of the present invention which comprises a polypeptide having the sequence of SEQ ID NO: 23 does not comprise: (i) a dengue antigen of serotype 2 which comprises SEQ ID NO: 2 or (ii) a dengue antigen comprising the prM-E sequence from SEQ ID NO: 2.

Preferably a dengue antigen of serotype 2 of the present invention further comprises a polypeptide selected from the group consisting of SEQ ID NO: 13, SEQ ID NO: 14, SEQ ID NO: 15, SEQ ID NO: 16 or SEQ ID NO: 18. For instance, said dengue antigen of serotype 2 preferably comprises: i) a polypeptide of SEQ ID NO: 13 and a polypeptide of SEQ ID NO: 19; ii) a polypeptide of SEQ ID NO: 14 and a polypeptide of SEQ ID NO: 20; iii) a polypeptide of SEQ ID NO: 15 and a polypeptide of SEQ ID NO: 21; iv) a polypeptide of SEQ ID NO: 16 and a polypeptide of SEQ ID NO: 22; or v) a polypeptide of SEQ ID NO: 18 and a polypeptide of SEQ ID NO: 23. When a vaccine composition of the present invention comprises a dengue antigen of serotype 2 which comprises a polypeptide having the sequence of SEQ ID NO: 13 and a polypeptide having the sequence of SEQ ID NO: 19, said vaccine composition is preferably either: (i) a vaccine composition comprising a dengue antigen comprising a polypeptide having the sequence of SEQ ID NO: 13 and a polypeptide having the sequence of SEQ ID NO: 19, wherein said vaccine composition does not comprise CYD-LAV or (ii) a vaccine composition comprising CYD-LAV. Said vaccine composition is also preferably either: (i) a vaccine composition comprising a dengue antigen comprising a polypeptide having the sequence of SEQ ID NO: 13 and a polypeptide having the sequence of SEQ ID NO: 19, wherein said vaccine composition does not comprise a dengue antigen comprising the prM and E sequences of CYD-LAV or (ii) a vaccine composition comprising a dengue antigen comprising the prM and E sequences of CYD-LAV. Said vaccine composition is also preferably either: (i) a vaccine composition comprising a dengue antigen comprising a polypeptide having the sequence of SEQ ID NO: 13 and a polypeptide having the sequence of SEQ ID NO: 19, wherein said vaccine composition does not comprise a dengue antigen comprising the M and E sequences of CYD-LAV or (ii) a vaccine composition comprising a dengue antigen comprising the M and E sequences of CYD-LAV. Said vaccine composition is also preferably either: (i) a vaccine composition comprising a dengue antigen comprising a polypeptide having the sequence of SEQ ID NO: 13 and a polypeptide having the sequence of SEQ ID NO: 19, wherein said vaccine composition does not comprise a chimeric dengue virus of serotype 2 generated using the prM-E sequence of LAV-2 or (ii) a vaccine composition comprising a chimeric dengue virus of serotype 2 generated using the prM-E sequence of LAV-2 (SEQ ID NO: 8). Preferably, a vaccine composition of the present invention which comprises a polypeptide having the sequence of SEQ ID NO: 13 and a polypeptide having the sequence of SEQ ID NO: 19 does not comprise: (i) a chimeric virus comprising the prM and E amino acid sequences of LAV-2; (ii) a dengue antigen of serotype 2 which comprises the prM-E sequence of CYD-LAV (SEQ ID NO: 8) or (iii) a dengue antigen comprising the prM-E sequence from LAV-2. When a vaccine composition of the present invention comprises a dengue antigen of serotype 2 which comprises a polypeptide having the sequence of SEQ ID NO: 15 and a polypeptide having the sequence of SEQ ID NO: 21, said vaccine composition is preferably either: (i) a vaccine composition comprising a dengue antigen comprising a polypeptide having the sequence of SEQ ID NO: 15 and a polypeptide having the sequence of SEQ ID NO: 21, wherein said vaccine composition does not comprise CYD-PR or (ii) a vaccine composition comprising CYD-PR. Said vaccine composition is also preferably either: (i) a vaccine composition comprising a dengue antigen a polypeptide having the sequence of SEQ ID NO: 15 and comprising a polypeptide having the sequence of SEQ ID NO: 21, wherein said vaccine composition does not comprise a dengue antigen comprising the prM and E sequences of CYD-PR or (ii) a vaccine composition comprising a dengue antigen comprising the prM and E sequences of CYD-PR. Said vaccine composition is also preferably either: (i) a vaccine composition comprising a dengue antigen comprising a polypeptide having the sequence of SEQ ID NO: 15 and a polypeptide having the sequence of SEQ ID NO: 21, wherein said vaccine composition does not comprise a chimeric dengue virus of serotype 2 generated using the prM-E sequence of PR/DB023 (SEQ ID NO: 10) or (ii) a vaccine composition comprising a chimeric dengue virus of serotype 2 generated using the prM-E sequence of PR/DB023 (SEQ ID NO: 10). Preferably, a vaccine composition of the present invention which comprises a polypeptide having the sequence of SEQ ID NO: 15 and a polypeptide having the sequence of SEQ ID NO: 21 does not comprise: (i) a chimeric virus comprising the prM and E amino acid sequences of PR/DB023; (ii) a dengue antigen of serotype 2 which comprises the prM-E sequence of CYD-PR (SEQ ID NO: 10) or (iii) a dengue antigen comprising the prM-E sequence from PR/DB023. When a vaccine composition of the present invention comprises a dengue antigen of serotype 2 which comprises a polypeptide having the sequence of SEQ ID NO: 16 and a polypeptide having the sequence of SEQ ID NO: 22, said vaccine composition is preferably either: (i) a vaccine composition comprising a dengue antigen comprising a polypeptide having the sequence of SEQ ID NO: 16 and a polypeptide having the sequence of SEQ ID NO: 22, wherein said vaccine composition does not comprise CYD-MD or (ii) a vaccine composition comprising CYD-MD. Said vaccine composition is also preferably either: (i) a vaccine composition comprising a polypeptide having the sequence of SEQ ID NO: 16 and a dengue antigen comprising a polypeptide having the sequence of SEQ ID NO: 22, wherein said vaccine composition does not comprise a dengue antigen comprising the prM and E sequences of CYD-MD or (ii) a vaccine composition comprising a dengue antigen comprising the prM and E sequences of CYD-MD. Said vaccine composition is also preferably either: (i) a vaccine composition comprising a dengue antigen comprising a polypeptide having the sequence of SEQ ID NO: 16 and a polypeptide having the sequence of SEQ ID NO: 22, wherein said vaccine composition does not comprise a dengue antigen comprising the M and E sequences of CYD-MD or (ii) a vaccine composition comprising a dengue antigen comprising the M and E sequences of CYD-MD. Said vaccine composition is also preferably either: (i) a vaccine composition comprising a dengue antigen comprising a polypeptide having the sequence of SEQ ID NO: 16 and a polypeptide having the sequence of SEQ ID NO: 22, wherein said vaccine composition does not comprise a chimeric dengue virus of serotype 2 generated using the prM-E sequence of MD1280 (SEQ ID NO: 11) or (ii) a vaccine composition comprising a chimeric dengue virus of serotype 2 generated using the prM-E sequence of MD1280 (SEQ ID NO: 11). Preferably, a vaccine composition of the present invention which comprises a polypeptide having the sequence of SEQ ID NO: 16 and a polypeptide having the sequence of SEQ ID NO: 22 does not comprise: (i) a chimeric virus comprising the prM and E amino acid sequences of MD1280; (ii) a dengue antigen of serotype 2 which comprises the prM-E sequence of CYD-MD (SEQ ID NO: 11) or (iii) a dengue antigen comprising the prM-E sequence from MD1280. When a vaccine composition of the present invention comprises a dengue antigen of serotype 2 which comprises a polypeptide having the sequence of SEQ ID NO: 18 and a polypeptide having the sequence of SEQ ID NO: 23, said vaccine composition is preferably either: (i) a vaccine composition comprising a dengue antigen comprising a polypeptide having the sequence of SEQ ID NO: 18 and a polypeptide having the sequence of SEQ ID NO: 23, wherein said vaccine composition does not comprise a chimeric dengue virus of serotype 2 generated using the prM-E sequence of SEQ ID NO: 2 or (ii) a vaccine composition comprising a chimeric dengue virus of serotype 2 generated using the prM-E sequence of SEQ ID NO: 2. Preferably, a vaccine composition of the present invention which comprises a polypeptide having the sequence of SEQ ID NO: 18 and a polypeptide having the sequence of SEQ ID NO: 23 does not comprise: (i) a dengue antigen of serotype 2 which comprises SEQ ID NO: 2 or (ii) a dengue antigen comprising the prM-E sequence from SEQ ID NO: 2.

A composition of the present invention, as described herein (e.g. a tetravalent formulation, e.g. for use a method of the present invention), may advantageously comprise a dengue antigen of serotype 2 which comprises a polypeptide having at least 90% identity to SEQ ID NO: 19, SEQ ID NO: 20, SEQ ID NO: 21, SEQ ID NO: 22 or SEQ ID NO: 23. Preferably said dengue antigen of serotype 2 further comprises a polypeptide having at least 90% identity to SEQ ID NO: 13, SEQ ID NO: 14, SEQ ID NO: 15, SEQ ID NO: 16 or SEQ ID NO: 18. For instance, said dengue antigen of serotype 2 preferably comprises: i) a polypeptide having at least 90% sequence identity to SEQ ID NO: 13 and a polypeptide having at least 90% sequence identity to SEQ ID NO: 19; ii) a polypeptide having at least 90% sequence identity to SEQ ID NO: 14 and a polypeptide having at least 90% sequence identity to SEQ ID NO: 20; iii) a polypeptide having at least 90% sequence identity to SEQ ID NO: 15 and a polypeptide having at least 90% sequence identity to SEQ ID NO: 21; iv) a polypeptide having at least 90% sequence identity to SEQ ID NO: 16 and a polypeptide having at least 90% sequence identity to SEQ ID NO: 22; or v) a polypeptide having at least 90% sequence identity to SEQ ID NO: 18 and a polypeptide having at least 90% sequence identity to SEQ ID NO: 23. In preferred embodiments, the references herein to at least 90% identity may be read as at least 91%, at least 92%, at least 93%, at least 94%, at least 95%, at least 96%, at least 97%, at least 98% or at least 99% identity to the given sequence.

The dengue antigens of serotype 2 as described in the preceding paragraphs may advantageously be combined with any of the dengue antigens of serotypes 1, 3 and 4 as described elsewhere herein to form a tetravalent formulation comprising a dengue antigen of serotype 1, a dengue antigen of serotype 2, a dengue antigen of serotype 3 and a dengue antigen of serotype 4. For instance, the dengue antigens of serotypes 1, 3 and 4 may each be independently selected from the group consisting of a live attenuated dengue virus, an inactivated dengue virus, a live attenuated or inactivated chimeric dengue virus or a dengue virus-like particle (VLP). Preferably, said dengue antigens of serotype 1, 3 and 4 are each independently selected from the group consisting of a live attenuated dengue virus and a live attenuated chimeric dengue virus. Preferably, said dengue antigens of serotypes 1, 3 and 4 are live attenuated chimeric dengue viruses of serotypes 1, 3 and 4 respectively. Preferably, said live attenuated chimeric dengue viruses of serotypes 1, 3 and 4 each comprise one or more proteins from a dengue virus and one or more proteins from a different flavivirus. For example, each of said live attenuated chimeric dengue viruses of serotypes 1, 3 and 4 is advantageously a YF/Dengue chimera. Preferably, said dengue antigens of serotypes 1, 3 and 4 are each a live attenuated chimeric dengue virus in which the genetic backbone of a recipient flavivirus has been modified by exchanging the sequences encoding the prM and E proteins of the recipient flavivirus with the corresponding sequences of a dengue virus. Preferably said recipient flavivirus is a yellow fever virus. For example, in an advantageous embodiment, said live attenuated chimeric dengue viruses of serotypes 1, 3 and 4 are respectively a Chimerivax dengue serotype 1 strain (i.e. a CYD-1 strain), a Chimerivax dengue serotype 3 strain (i.e. a CYD-3 strain) and a Chimerivax dengue serotype 4 strain (i.e. a CYD-4 strain).

It is an aim of the present inventors to provide an optimized tetravalent dengue vaccine composition (i.e. vaccine composition comprising a dengue antigen of each of serotypes 1, 2, 3 and 4) which provides an improved neutralising antibody response against dengue virus of serotype 2 when compared with the neutralising antibody response generated by CYD-1, CYD-2, CYD-3 and CYD-4 as defined in Example 1.

Accordingly, in one aspect, the present invention advantageously provides a vaccine composition which comprises a dengue antigen of each of serotypes 1, 2, 3 and 4, wherein said dengue antigens of serotypes 1, 3 and 4 are each a live attenuated chimeric dengue virus and said dengue antigen of serotype 2 is a live attenuated dengue virus which comprises a nucleic acid sequence having at least 90% sequence identity to the sequence as set forth in SEQ ID NO: 24.

Accordingly, in another aspect, the present invention advantageously provides a vaccine composition which comprises a dengue antigen of serotype 1, a dengue antigen of serotype 2, a dengue antigen of serotype 3 and a dengue antigen of serotype 4, wherein:
i) said dengue antigen of serotype 1 is a YF/dengue chimeric dengue virus (i.e. a recipient yellow fever virus in which the genetic backbone of the YF virus has been modified by exchanging the sequences encoding the prM and E proteins of the YF virus by the corresponding sequences of a dengue serotype 1 virus);
ii) said dengue antigen of serotype 2 is a live attenuated dengue virus of serotype 2 which comprises a nucleic acid sequence having at least 90% sequence identity to the sequence as set forth in SEQ ID NO: 24;
iii) said dengue antigen of serotype 3 is a YF/dengue chimeric dengue virus (i.e. a recipient yellow fever virus in which the genetic backbone of the YF virus has been modified by exchanging the sequences encoding the prM and E proteins of the YF virus by the corresponding sequences of a dengue serotype 3 virus) and
iv) said dengue antigen of serotype 4 is a YF/dengue chimeric dengue virus (i.e. a recipient yellow fever virus in which the genetic backbone of the YF virus has been modified by exchanging the sequences encoding the prM and E proteins of the YF virus by the corresponding sequences of a dengue serotype 4 virus).

Preferably, said recipient YF virus (which forms the genetic backbone of the YF/dengue chimeric viruses of serotypes 1, 3 and 4) is an attenuated YF virus. For example, said recipient YF virus may be an attenuated YF virus selected from the group consisting of YF 17D, YF 17DD and YF 17D204. Preferably, the YF/dengue chimeric viruses of serotypes 1, 3 and 4 are respectively a Chimerivax dengue serotype 1 (i.e. a CYD-1), a Chimerivax dengue serotype 3 (i.e. a CYD-3) and a Chimerivax dengue serotype 4 (i.e. a CYD-4).

A reference herein to a nucleic acid sequence having at least 90% sequence identity to the sequence as set forth in SEQ ID NO: 24 may preferably be read as a nucleic acid sequence having at least 92%, at least 94%, at least 96%, at least 98%, at least 99% or 100% sequence identity to the sequence as set forth in SEQ ID NO: 24. Preferably the nucleotides at the positions within said nucleic acid sequences (that have at least 90% sequence identity to the sequence as set forth in SEQ ID NO: 24) which correspond to positions 736, 1619, 4723, 5062, 9191, 10063, 10507, 57, 524, 2055, 2579, 4018, 5547, 6599 and 8571 of SEQ ID NO: 24 are not mutated. Advantageously, a dengue antigen of serotype 2 which is a live attenuated dengue virus for use in a composition of the present invention (for example for use in combination with a dengue antigen of serotypes 1, 3 and 4 as described above and elsewhere herein (e.g. dengue antigens of serotypes 1, 3 and 4 which are live attenuated chimeric dengue viruses, e.g. YF/dengue chimeric dengue viruses)) is a live attenuated dengue virus which comprises a nucleic acid sequence having 100% sequence identity to the sequence as set forth in SEQ ID NO: 24 or a live attenuated dengue virus which comprises at least one and no more than 20 nucleotide substitutions when compared with the sequence as set forth in SEQ ID NO: 24. Preferably said live attenuated dengue virus comprises at least one and no more than 15, 14, 13, 12, 11, 10, 9, 8, 7, 6, 5, 4, 3 or 2 nucleotide substitutions when compared with the sequence as set forth in SEQ ID NO: 24. Preferably the nucleotides at the positions within said nucleic acid sequences which correspond to positions 736, 1619, 4723, 5062, 9191, 10063, 10507, 57, 524, 2055, 2579, 4018, 5547, 6599 and 8571 of SEQ ID NO: 24 are not mutated. Advantageously, a dengue antigen of serotype 2 which is a live attenuated dengue virus for use in a composition of the present invention comprises a nucleic acid sequence that has no more than 20 base mutations, deletions or insertions when compared with the sequence as set forth in SEQ ID NO: 24. In certain cases said live attenuated dengue virus of serotype 2 comprises a nucleic acid sequence that has no more than 15 or even no more than 12, 11, 10, 9, 8, 7, 6, 5, 4, 3, 2 or 1 base mutations, deletions or insertions when compared with the sequence as set forth in SEQ ID NO: 24. Preferably the nucleotides at the positions within said nucleic acid sequence that correspond to positions 736, 1619, 4723, 5062, 9191, 10063, 10507, 57, 524, 2055, 2579, 4018, 5547, 6599 and 8571 of SEQ ID NO: 24 are not mutated.

It is also preferred that a dengue antigen of serotype 2 for use in a vaccine composition of the present invention (e.g. a dengue antigen which is a live attenuated dengue virus or a live attenuated chimeric dengue virus of serotype 2) is capable of inducing neutralizing antibodies in humans and is capable of inducing a balanced immune response when used in the context of a tetravalent dengue vaccine composition. It is also preferred that a dengue antigen of serotype 2 for use in a vaccine composition of the present invention (e.g. a dengue antigen which is a live attenuated dengue virus or a live attenuated chimeric dengue virus of serotype 2) for use in a vaccine composition of the invention results in low or absent viremia in humans. It is also preferred that a dengue antigen of serotype 2 for use in a tetravalent vaccine composition of the present invention (e.g. a dengue antigen which is a live attenuated dengue virus or a live attenuated chimeric dengue virus of serotype 2) provides an improved neutralising antibody response against dengue virus of serotype 2 when compared with the neutralising antibody response generated by CYD-1, CYD-2, CYD-3 and CYD-4 as defined in Example 1.

Advantageously, a composition for use in the present invention comprises a dengue antigen of each of serotypes 1, 2, 3 and 4, wherein: (i) said dengue antigen of serotype 1 is a live attenuated chimeric dengue virus other than CYD-1 or said dengue antigen of serotype 1 is CYD-1; (ii) said dengue antigen of serotype 2 is a live attenuated dengue virus other than VDV-2 or said dengue antigen of serotype 2 is VDV-2; (iii) said dengue antigen of serotype 3 is a live attenuated chimeric dengue virus other than CYD-3 or said dengue antigen of serotype 3 is CYD-3 and (iv) said dengue antigen of serotype 4 is a live attenuated chimeric dengue virus other than CYD-4 or said dengue antigen of serotype 4 is CYD-4. In this context, the VDV-2 strain is the strain derived from the DEN-2 16681/PDK53 strain (LAV2) by subsequent adaptation to Vero cells, wherein said VDV-2 strain has 10 additional mutations in comparison with the DEN-2 16681/PDK53 strain including four silent mutations.

Advantageously, a composition for use in the present invention comprises a dengue antigen of each of serotypes 1, 2, 3 and 4, wherein said dengue antigens of serotypes 1, 3 and 4 are each a live attenuated chimeric dengue virus and said dengue antigen of serotype 2 is a live attenuated dengue virus which comprises a nucleic acid sequence having at least 90% sequence identity to the sequence as set forth in SEQ ID NO: 24 and wherein said dengue antigens of serotypes 1, 2, 3 and 4 are not CYD-1, VDV-2, CYD-3 and CYD-4 respectively or a dengue antigen comprising the M and E sequences of CYD-1, VDV2, a dengue antigen comprising the M and E sequences of CYD-3 and a dengue antigen comprising the M and E sequences of CYD-4 respectively.

Advantageously, a composition for use in the present invention comprises a dengue antigen of each of serotypes 1, 2, 3 and 4, wherein: (i) said dengue antigen of serotype 1 is a live attenuated chimeric dengue virus other than CYD-1 or said dengue antigen of serotype 1 is CYD-1; (ii) said dengue antigen of serotype 2 is a live attenuated dengue virus other than VDV-2 or said dengue antigen of serotype 2 is VDV-2; (iii) said dengue antigen of serotype 3 is a live attenuated chimeric dengue virus other than CYD-3 or said dengue antigen of serotype 3 is CYD-3 and (iv) said dengue antigen of serotype 4 is a live attenuated chimeric dengue virus other than CYD-4 or said dengue antigen of serotype 4 is CYD-4. In this context, the VDV-2 strain is the strain comprising the nucleic acid sequence as set forth in SEQ ID NO: 24.

A preferred vaccine composition according to the present invention, e.g. for use in a method according to the present invention, comprises a dengue antigen of serotype 1, a dengue antigen of serotype 2, a dengue antigen of serotype 3 and a dengue antigen of serotype 4, wherein:
i) said dengue antigen of serotype 1 is a YF/dengue chimeric dengue virus other than a CYD-1 or said dengue antigen of serotype 1 is a CYD-1;
ii) said dengue antigen of serotype 2 is a live attenuated dengue virus of serotype 2 which comprises a nucleic acid sequence having at least 90% sequence identity to the sequence as set forth in SEQ ID NO: 24, wherein said dengue antigen of serotype 2 is not a live attenuated dengue virus of serotype 2 which comprises a nucleic acid sequence having 100% sequence identity to the sequence as set forth in SEQ ID NO: 24 or said dengue antigen of serotype 2 is a live attenuated dengue virus of serotype 2 which comprises a nucleic acid sequence having 100% sequence identity to the sequence as set forth in SEQ ID NO: 24;
iii) said dengue antigen of serotype 3 is a YF/dengue chimeric dengue virus other than a CYD-3 or said dengue antigen of serotype 3 is a CYD-3; and
iv) said dengue antigen of serotype 4 is a YF/dengue chimeric dengue virus other than a CYD-4 or said dengue antigen of serotype 4 is a CYD-4.

Advantageously, a dengue antigen of serotype 2 which is a live attenuated chimeric dengue virus for use in a vaccine composition of the present invention (for example for use in combination with a dengue antigen of serotypes 1, 3 and 4 as described above and elsewhere herein (e.g. dengue antigens of serotypes 1, 3 and 4 which are YF/dengue chimeric dengue viruses)) comprises a nucleic acid sequence having at least 90% identity to the sequence as set forth in SEQ ID NO: 25. Preferably said nucleic acid sequence has at least 92%, at least 94%, at least 96%, at least 98%, at least 99% or 100% sequence identity to the sequence as set forth in SEQ ID NO: 25. Preferably the nucleotides at the positions within said nucleic acid sequence which correspond to positions 524, 736, 1619 and 2055 of SEQ ID NO: 24 are not mutated (i.e. maintain the nucleotide appearing in SEQ ID NO: 24 at those positions).

Advantageously, a dengue antigen of serotype 2 which is a chimeric dengue virus for use in a vaccine composition of the present invention (for example for use in combination with a dengue antigen of serotypes 1, 3 and 4 as described above and elsewhere herein (e.g. dengue antigens of serotypes 1, 3 and 4 which are YF/dengue chimeric dengue viruses)) comprises a prM-E sequence having at least 90%, at least 95%, at least 98%, at least 99% or 100% identity to the prM-E sequence from the LAV-2 strain (i.e. the RNA equivalent of SEQ ID NO: 4). Preferably the nucleotides at the positions within said prM-E sequence which correspond to positions 524, 736, 1619 and 2055 of the RNA equivalent of SEQ ID NO: 24 are not mutated (i.e. maintain the nucleotide appearing in the RNA equivalent of SEQ ID NO: 24 at those positions).

Advantageously, a dengue antigen of serotype 2 which is a chimeric dengue virus for use in a vaccine composition of the present invention (for example for use in combination with a dengue antigen of serotypes 1, 3 and 4 as described above and elsewhere herein (e.g. dengue antigens of serotypes 1, 3 and 4 which are YF/dengue chimeric dengue viruses)) comprises a prM-E sequence having at least 90%, at least 95%, at least 98%, at least 99% or 100% identity to the prM-E sequence from the MD1280 strain (i.e. the RNA equivalent of SEQ ID NO: 7).

A composition of the present invention, as described herein, may advantageously comprise a dengue antigen selected from the group consisting of: (a) a live attenuated dengue virus; (b) an inactivated dengue virus; (c) a live attenuated or inactivated chimeric dengue virus and (d) a combination of two or more of (a) to (c), wherein said dengue antigen comprises a nucleotide sequence selected from the group consisting of SEQ ID NO: 4, SEQ ID NO: 5, SEQ ID NO: 6, SEQ ID NO: 7 and SEQ ID NO: 1.

A composition of the present invention, as described herein, may advantageously comprise a dengue antigen selected from the group consisting of: (a) a live attenuated dengue virus; (b) an inactivated dengue virus; (c) a live attenuated or inactivated chimeric dengue virus and (d) a combination of two or more of (a) to (c), wherein said dengue antigen comprises a nucleotide sequence encoding M and E sequences as described herein.

For instance, a composition of the present invention, e.g. for use in a method of protecting according to the present invention, may advantageously comprise any one of the following combinations of dengue antigens of serotypes 1, 2, 3 and 4: i) CYD-1, CYD-LAV, CYD-3 and CYD-4; ii) CYD-1, CYD-BID, CYD-3 and CYD-4; (iii) CYD-1, CYD-PR, CYD-3 and CYD-4 or (iv) CYD-1, CYD-MD, CYD-3 and CYD-4. A composition of the present invention may also advantageously comprise the following combination of dengue antigens: i) a dengue antigen comprising the prM and E sequences of CYD-1, VDV2, a dengue antigen comprising the prM and E sequences of CYD-3 and a dengue antigen comprising the prM and E sequences of CYD-4. For instance, a composition of the present invention may advantageously comprise CYD-1, VDV-2, CYD-3 and CYD-4. A composition of the present invention, as described herein, may advantageously comprise a dengue antigen of serotype 2 which comprises the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. A composition of the present invention, as described herein, may advantageously comprise a dengue antigen of serotype 2 which comprises a sequence having at least 90% identity to the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. For example, said sequence may be at least 91%, at least 92%, at least 93%, at least 94%, at least 95%, at least 96%, at least 97%, at least 98% or at least 99% identical to the prm-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2.

A vaccine composition for use the present invention, e.g. for use in a method according to the present invention preferably comprises a dengue antigen which is a vaccinal dengue virus. Such vaccinal dengue viruses include, for example, inactivated viruses, live attenuated viruses and live attenuated chimeric dengue viruses. Preferably, the vaccinal dengue viruses are live attenuated chimeric dengue viruses. Preferably, a live attenuated chimeric dengue virus according to the present invention comprises one or more proteins from a dengue virus and one or more proteins from a different flavivirus. Advantageously, said different flavivirus is a yellow fever virus, for example a yellow fever virus of strain YF 17D. Preferably a chimeric dengue virus according to the present invention comprises the prM-E amino acid sequences of a dengue virus, for example a chimeric dengue virus according to the present invention comprises a yellow fever virus genome whose prM-E whose prM-E sequence has been substituted with the prM-E sequence of a dengue virus. Advantageously, a vaccine composition according to the present invention, e.g. for use in a method of the present invention, comprises CYD-1, CYD-2, CYD-3 and CYD-4. A composition of the present invention may advantageously comprise any one of the following combinations of dengue antigens i) a dengue antigen comprising the prM and E sequences of CYD-1, a dengue antigen comprising the prM and E sequences of CYD-LAV, a chimeric dengue virus comprising the prM and E sequences of CYD-3 and a dengue antigen comprising the prM and E sequences of CYD-4; ii) a dengue antigen comprising the prM and E sequences of CYD-1, a dengue antigen comprising the prM and E sequences of CYD-BID, a dengue antigen comprising the prM and E sequences of CYD-3 and a dengue antigen comprising the prM and E sequences of CYD-4; (iii) a dengue antigen comprising the prM and E sequences of CYD-1, a dengue antigen comprising the prM and E sequences of CYD-PR, a dengue antigen comprising the prM and E sequences of CYD-3 and a dengue antigen comprising the prM and E sequences of CYD-4; (iv) a dengue antigen comprising the prM and E sequences of CYD-1, a dengue antigen comprising the prM and E sequences of CYD-MD, a dengue antigen comprising the prM and E sequences of CYD-3 and a dengue antigen comprising the prM and E sequences of CYD-4;. For instance, a composition of the present invention may also advantageously comprise any one of the following combinations of dengue antigens: i) CYD-1, CYD-LAV, CYD-3 and CYD-4; ii) CYD-1, CYD-BID, CYD-3 and CYD-4; (iii) CYD-1, CYD-PR, CYD-3 and CYD-4 or (iv) CYD-1, CYD-MD, CYD-3 and CYD-4. A composition of the present invention may also advantageously comprise the following combination of dengue antigens: i) a dengue antigen comprising the prM and E sequences of CYD-1, VDV2, a dengue antigen comprising the prM and E sequences of CYD-3 and a dengue antigen comprising the prM and E sequences of CYD-4. For instance, a composition of the present invention may advantageously comprise CYD-1, VDV-2, CYD-3 and CYD-4. A composition of the present invention, as described herein, may advantageously comprise a dengue antigen of serotype 2 which comprises the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. Advantageously, a vaccine composition of the present invention, e.g. a chimeric dengue virus, of serotype 2 may comprise prM-E sequences having at least 90%, at least 95%, at least 98% or at least 99% identity to the prM-E sequences from the serotype 2 strains LAV-2, BID-V585, PR/DB023 or MD1280 as described in the examples or may comprise prM-E sequences having at least 90%, at least 95%, at least 98% or at least 99% identity to the prM-E sequence shown in SEQ ID NO: 2. Advantageously, a vaccine composition, e.g. a chimeric dengue virus, of serotype 2 for use in the method of the present invention may comprise prM-E sequences from the serotype 2 strains LAV-2, BID-V585, PR/DB023 or MD1280 or the prM-E sequence from SEQ ID NO: 2 as described in the examples. When the recipient genomic backbone of such chimeric dengue viruses is derived from YF-VAX®, such strains are referred to herein as CYD-LAV, CYD-BID, CYD-PR and CYD-MD. A vaccine composition of the present invention comprising chimeric dengue virus of serotype 2 generated using the prM-E sequences of the serotype 2 strains LAV-2 (SEQ ID NO: 8), BID-V585 (SEQ ID NO: 9), PR/DB023 (SEQ ID NO: 10), MD1280 (SEQ ID NO: 11) or SEQ ID NO: 2, or generated using prM-E sequences having at least 90%, at least 95%, at least 98% or at least 99% identity to the prM-E sequences from the serotype 2 strains LAV-2, BID-V585, PR/DB023, MD1280 or the prM-E sequence from SEQ ID NO: 2 may advantageously be used in combination with CYD-1, CYD-3 and CYD-4 in a vaccine composition according to the present invention. A composition of the present invention, as described herein, may advantageously comprise a dengue antigen of serotype 2 which comprises a sequence having at least 90% identity to the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2. For example, said sequence may be at least 91%, at least 92%, at least 93%, at least 94%, at least 95%, at least 96%, at least 97%, at least 98% or at least 99% identical to the prM-E sequence of CYD-LAV (SEQ ID NO: 8), CYD-BID (SEQ ID NO: 9), CYD-PR (SEQ ID NO: 10) CYD-MD (SEQ ID NO: 11) or SEQ ID NO: 2.

The exact quantity of a vaccinal dengue virus of the present invention to be administered may vary according to the age and the weight of the patient being vaccinated, the frequency of administration as well as the other ingredients (e.g. adjuvants) in the composition. The quantity of a live attenuated dengue virus of each of serotypes 1 to 4 comprised in a vaccine composition of the present invention lies within a range of from about 10³ to about 10⁷ CCID₅₀. Generally, the quantity of a live attenuated dengue virus of each of serotypes 1 to 4 comprised in a vaccine composition of the present invention lies within a range of from about 10³ to about 10⁶ CCID₅₀, for example within a range of from about 5×10³ to about 5×10⁵ CCID₅₀, for example within a range of from about 1×10⁴ to about 1×10⁵ CCID₅₀, for example about 10⁵ CCID₅₀. The quantity of a live attenuated dengue virus of each of serotypes 1 to 4 comprised in a vaccine composition of the present invention may also lie within a range of from about 10⁴ to about 10⁷ CCID₅₀, for example about 10⁶ CCID₅₀. The quantity of a live attenuated dengue virus of each of serotypes 1 to 4 comprised in a tetravalent composition of the present invention may be equal. For example a tetravalent composition of the present invention may comprise about 10⁵ CCID₅₀ of each live attenuated dengue virus of serotypes 1 to 4. Alternatively, a tetravalent composition of the present invention may comprise about 10⁶ CCID₅₀ of each live attenuated dengue virus of serotypes 1 to 4. Generally, the quantity of an inactivated dengue virus of each of serotypes 1 to 4 comprised in a composition of the present invention lies within a range of from about 10⁴ to about 10⁸ CCID₅₀ equivalent, preferably within a range of from about 5×10⁴ to about 5×10⁷ CCID₅₀ equivalent, preferably within a range of from about 1×10⁴ to about 1×10⁶ CCID₅₀ equivalent, preferably about 10⁵ CCID₅₀ equivalent. Generally, the quantity of a VLP of each of serotypes 1 to 4 comprised in the composition lies within a range of from about 100 ng to about 100 μg of VLP, preferably within a range of from about 100 ng to about 50 μg, preferably within a range of from about 100 ng to about 20 μg, preferably about 1 μg to 10 μg. The amount of VLP can be determined by ELISA. Advantageously, a vaccine composition according to the present invention comprises an effective amount of a dengue antigen as defined herein.

A vaccine composition according to the present invention may further comprise a pharmaceutically acceptable carrier or excipient. A pharmaceutically acceptable carrier or excipient according to the present invention means any solvent or dispersing medium etc., commonly used in the formulation of pharmaceuticals and vaccines to enhance stability, sterility and deliverability of the active agent and which does not produce any secondary reaction, for example an allergic reaction, in humans. The excipient is selected on the basis of the pharmaceutical form chosen, the method and the route of administration. Appropriate excipients, and requirements in relation to pharmaceutical formulation, are described in “Remington's Pharmaceutical Sciences” (19th Edition, A.R. Gennaro, Ed., Mack Publishing Co., Easton, Pa. (1995)). Particular examples of pharmaceutically acceptable excipients include water, phosphate-buffered saline (PBS) solutions and a 0.3% glycine solution. A vaccine composition according to the present invention may advantageously comprise 0.4% saline and 2.5% human serum albumin (HSA).

A vaccine composition for use in a method of the present invention may optionally contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, human serum albumin, essential amino acids, nonessential amino acids, L-arginine hydrochlorate, saccharose, D-trehalose dehydrate, sorbitol, tris (hydroxymethyl) aminomethane and/or urea. In addition, the vaccine composition may optionally comprise pharmaceutically acceptable additives including, for example, diluents, binders, stabilizers, and preservatives. Preferred stabilizers are described in WO 2010/003670.

A vaccine composition of the present invention may comprise a dengue antigen which is a dengue immunoprotein. A dengue immunoprotein, as used herein, is a dengue envelope (E) protein, or derivative or fragment thereof, that when administered to an immunocompetent subject induces neutralizing antibodies against a dengue virus of serotype 1, 2, 3 or 4. Dengue immunoproteins include native and derivatized forms of dengue E proteins, including chemical conjugates, immunological fragments, and fusion proteins thereof.

Dengue immunoproteins, or derivatives or fragments thereof may be conjugated to carrier molecules. Such conjugation may be achieved by chemical conjugation techniques or through the recombinant expression of fusion proteins comprising the dengue immunoproteins or derivatives or fragments thereof and the carrier molecule. Examples of carrier molecules which may be used for preparing conjugates include diphtheria toxoid, tetanus toxoid, fragment C of tetanus toxin, mutants of diphtheria toxin including CRM 197, CRM 176, CRM228, CRM 45, CRM 9, CRM 45, CRM 102, CRM 103 and CRM 107, pneumococcal pneumolysin, OMPC, heat shock proteins, pertussis proteins, pneumococcal surface protein PspA or the toxin A or B of Clostridium difficile.

A vaccine composition of the present invention may comprise one or more adjuvants to enhance the immunogenicity of the dengue antigens. Those skilled in the art will be able to select an adjuvant which is appropriate in the context of this invention. An adjuvant is preferably used in a vaccine composition of the invention comprising an inactivated virus or a VLP or a dengue structural protein. An adjuvant may be used in a vaccine composition of the invention comprising a live attenuated virus, as long as said adjuvant does not impact replication.

Suitable adjuvants include an aluminum salt such as aluminum hydroxide gel, aluminum phosphate or alum, but may also be a salt of calcium, magnesium, iron or zinc. Further suitable adjuvants include an insoluble suspension of acylated tyrosine or acylated sugars, cationically or anionically derivatized saccharides, or polyphosphazenes. Alternatively, the adjuvant may be an oil-in-water emulsion adjuvant (EP 0 399 843B), as well as combinations of oil-in-water emulsions and other active agents (WO 95/17210; WO 98/56414; WO 99/12565 and WO 99/11241). Other oil emulsion adjuvants have been described, such as water-in-oil emulsions (U.S. Pat. No. 5,422, 109; EP 0 480 982 B2) and water-in-oil-in-water emulsions (U.S. Pat. No. 5,424,067; EP 0 480 981 B). Examples of such adjuvants include MF59, AF03 (WO 2007/006939), AF04 (WO 2007/080308), AF05, AF06 and derivatives thereof. The adjuvant may also be a saponin, lipid A or a derivative thereof, an immunostimulatory oligonucleotide, an alkyl glucosamide phosphate, an oil in water emulsion or combinations thereof. Examples of saponins include Quil A and purified fragments thereof such as QS7 and QS21.

As appreciated by skilled artisans, a vaccine composition of the present invention is suitably formulated to be compatible with the intended route of administration. Examples of suitable routes of administration include for instance intramuscular, transcutaneous, subcutaneous, intranasal, oral or intradermal. Advantageously, the route of administration is subcutaneous.

The vaccine compositions of the present invention may be administered using conventional hypodermic syringes or safety syringes such as those commercially available from Becton Dickinson Corporation (Franklin Lakes, N.J., USA) or jet injectors. For intradermal administration, conventional hypodermic syringes may be employed using the Mantoux technique or specialized intradermal delivery devices such as the BD Soluvia™ microinjection system (Becton Dickinson Corporation, Franklin Lakes, N.J., USA), may be used.

The volume of a vaccine composition of the present invention administered will depend on the method of administration. In the case of subcutaneous injections, the volume is generally between 0.1 and 1.0 ml, preferably approximately 0.5 ml.

Optionally, booster administrations of a vaccine composition according to the present invention may be used, for example between six months and ten years, for example six months, one year, three years, five years or ten years after initial immunization (i.e. after administration of the last dose scheduled in the initial immunization regimen).

According to one embodiment, the invention also provides a kit comprising a vaccine composition of the invention and instructions for the use of said vaccine composition in a method of protecting a human subject against dengue disease. The kit can comprise at least one dose (typically in a syringe) of any vaccine composition contemplated herein. According to one embodiment the kit may comprises a multi-dose formulation (typically in a vial) of any vaccine composition as described herein. The kit further comprises a leaflet mentioning the use of the said vaccine composition for the prevention of dengue disease or the use of the said vaccine for the prophylaxis of dengue disease. The leaflet may further mention the vaccination regimen and the human subject population to be vaccinated.

The efficacy of a vaccine composition of the present invention in reducing the likelihood or severity of dengue disease may be measured in a number of ways. For instance the efficacy of a vaccine composition of the present invention in reducing the likelihood or severity of symptomatic virologically-confirmed dengue disease may be calculated by measuring after the administration of at least one dose of said vaccine composition (e.g. after administration of one, two or three doses of said vaccine composition):
(i) the percentage of symptomatic virologically-confirmed dengue cases caused by dengue virus of any serotype;
(ii) the percentage of severe virologically-confirmed dengue cases caused by dengue virus of any serotype;
(iii) the percentage of dengue hemorrhagic fever cases of Grades I to IV caused by dengue virus of any serotype;
(iv) the percentage of DHF cases of Grade I caused by dengue virus of any serotype;
(v) the percentage of DHF cases of Grade II caused by dengue virus of any serotype;
(vi) the percentage of DHF cases of Grade III caused by dengue virus of any serotype;
(vii) the percentage of DHF cases of Grade IV caused by dengue virus of any serotype;
(viii) the annual incidence rate of hospitalized virologically-confirmed dengue caused by dengue virus of any serotype; and/or
(ix) the length of hospital stay for symptomatic virologically-confirmed dengue cases caused by dengue virus of any serotype;
   in a group of subjects that has received said vaccine composition and comparing those measurements with the equivalent measurements from a control group of subjects that has not received said vaccine composition, wherein the subjects in both said groups are resident in a Dengue endemic region. A statistically significant reduction in any one or more of (i) to (ix) in the vaccinated group of subjects when compared with the unvaccinated control group of subjects is indicative of the efficacy of a vaccine composition according to the present invention. In a preferred embodiment, the efficacy of a vaccine composition according to the present invention is demonstrated by a statistically significant reduction of one or more of the measures as described above, wherein the DHF cases or dengue cases are caused by dengue virus of serotypes 1, 3 or 4.

The efficacy of a vaccine composition according to the present invention in reducing the severity or likelihood of dengue disease may also be calculated by measuring after the administration of at least one dose of said vaccine composition (e.g. after administration of one, two or three doses of said vaccine composition):
(i) the mean duration and/or intensity of fever;
(iii) the mean value for plasma leakage as defined by a change in haematocrit;
(iii) the mean value for thrombocytopenia (platelet count); and/or
(iv) the mean value of the level of liver enzymes including alanine aminotransferase (ALT) and aspartate aminotransferase (AST);
   in a group of subjects that has received said vaccine composition and who have developed virologically-confirmed dengue disease and comparing those measurements with the equivalent measurements from a control group of subjects that has not received said vaccine composition and who have developed virologically-confirmed dengue disease. A statistically significant reduction in any one or more of (i) to (v) in the vaccinated group of subjects who have developed virologically-confirmed dengue disease when compared with the control group of subjects who have developed virologically-confirmed dengue disease is indicative of the efficacy of a vaccine composition according to the present invention in reducing the severity or likelihood of dengue disease.

Typically the efficacy of the method of protection of the invention against a dengue disease, as measured e.g. by the method described in example 1 (VE=100*(1−ID_{CYD}/ID_{control}), where ID is the incidence density (i.e., the number of human subjects with virologically-confirmed dengue divided by the number of person-years at risk) in each group), is at least 50%, preferably at least 60%, wherein said dengue disease is caused by serotype 1, 3 or 4. The efficacy of the method of protection being advantageously at least 70%, preferably 80% against a dengue disease caused by serotype 3 or 4. The efficacy of the method of protection being advantageously at least 90% against dengue disease caused by serotype 4.

Percent identity between two amino acid sequences or two nucleotide sequences is determined by standard alignment algorithms such as, for example, Basic Local Alignment Tool (BLAST) described in Altschul et al. (1990) J. Mol. Biol., 215: 403-410, the algorithm of Needleman et al. (1970) J. Mol. Biol., 48: 444-453; the algorithm of Meyers et al. (1988) Comput. Appl. Biosci., 4: 11-17; or Tatusova et al. (1999) FEMS Microbiol. Lett., 174: 247-250, etc. Such algorithms are incorporated into the BLASTN, BLASTP and “BLAST 2 Sequences” programs (see www.ncbi.nim.nih.gov/BLAST). When utilizing such programs, the default parameters can be used. For example, for nucleotide sequences the following settings can be used for “BLAST 2 Sequences”: program BLASTN, reward for match 2, penalty for mismatch-2, open gap and extension gap penalties 5 and 2 respectively, gap x˜dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x˜dropoff 50, expect 10, word size 3, filter ON.

It is understood that the various features and preferred embodiments of the present invention as disclosed herein may be combined together.

Throughout this application, various references are cited. The disclosures of these references are hereby incorporated by reference into the present disclosure.

The present invention will be further illustrated by the following examples. It should be understood however that the invention is defined by the claims, and that these examples are given only by way of illustration of the invention and do not constitute in any way a limitation thereof.

### EXAMPLES

### Example 1

### One Year Follow-Up in Thailand of Patients Vaccinated with a Tetravalent Dengue Vaccine (TDV) Composition comprising Chimerivax™ DEN-1, DEN-2, DEN-3 and DEN-4

### Methods

### Study Design and Participants

An observer-blind, randomised, controlled, monocentre, Phase IIb trial of the efficacy of the tetravalent Chimerivax™ vaccine (i.e. a tetravalent vaccine comprising the particular CYD-1 strain generated from the prM and E sequences of DEN1 PU0359 (TYP 1 140), the particular CYD-2 strain generated from the prM and E sequences of DEN2 PU0218, the particular CYD-3 strain generated from the prM and E sequences of DEN3 PaH881/88 and the particular CYD-4 strain generated from the prM and E sequences of DEN4 1228 (TVP 980), see WO 03/101397 and Guy et al., Vaccine (2011), 29(42): 7229-41) against virologically-confirmed dengue disease is conducted. 4002 schoolchildren aged 4-11 years who are in good health based on medical history and physical examination are enrolled into the trial. The study is conducted at Ratchaburi Regional Hospital (RRH), Ratchaburi province, Muang district, Thailand. Children with acute febrile illness at enrolment, those with congenital or acquired immunodeficiency, and those receiving immunosuppressive therapy or other prohibited treatments or vaccines are excluded. Participants are randomly assigned 2:1 to receive three doses of dengue vaccine or a control product at Months 0, 6 and 12.

### Products

Each of the chimeric viruses are produced and cultured on Vero cells as described in WO 03/10197, Guy et al, Hum. Vaccines (2010) 6 (9): 696; Guy et al, Vaccine (2010) 28: 632; Guirakhoo et al, J. Virol. (2000) 74: 5477; Guirakhoo et al, J. Virol. (2001) 75 (16): 7290; Guirakhoo et al, Virol. (June 20, 2002) 298: 146; and Guirakhoo et al, J. Virol. (2004) 78 (9): 4761.

The vaccine is presented as a lyophilized powder (previously stored at temperature of between 2° C. and 8° C.), which is reconstituted with 0.5 mL of solvent for injection (0.4% NaCl containing 2.5% human serum albumin). As reconstituted, each 0.5 mL dose of vaccine contains 5±1 log₁₀ CCID₅₀ of each chimeric dengue serotype (1, 2, 3 and 4) and excipients (essential amino acids, non-essential amino acids, L-arginine chlorhydrate, saccharose, D-trehalose dehydrate, sorbitol, tris (hydroxymethyl) aminoethane and urea). The control product is inactivated rabies vaccine (Verorab®, Sanofi Pasteur, Lyon France) for the first injection of the first 50 children randomised to the control group, and 0.9% NaCI saline placebo for all other injections. All products are injected subcutaneously into the deltoid region of the upper arm.

### Assessments

All children are actively followed to detect acute febrile illness based on daily surveillance of school registers during school terms for absenteeism (followed by phone calls or home visits to absentees), and twice-weekly home visits, phone calls or mobile phone text-messages throughout school holidays. In any case of febrile illness (defined as illness with two temperature readings of ≧37.5° C. at least 4 hours apart) parents are asked to take their child to RRH for diagnosis and treatment. The surveillance system also captures spontaneous consultations at RRH. Consecutive febrile episodes separated by a symptom-free interval of ≧14 days are considered as separate episodes. Paired serum samples are collected at presentation (i.e., acute sample, collected no later than 7 days after fever onset) and 7-14 days later (convalescent sample) and sent to Sanofi Pasteur's Global Clinical Immunology (GCI) laboratory (Swiftwater, Pa., USA) and to the Centre for Vaccine Development (CVD, Mahidol University, Thailand). Acute samples are screened for the presence of flavivirus using an initial RT-PCR assay which detects the presence of any flavivirus (using primers composed of highly conserved flavivirus sequences). Positive samples are tested for wild-type dengue virus with a serotype-specific quantitative RT-PCR, as described herein. In parallel, all acute samples are tested for the presence of dengue NS1 antigen using commercial ELISA kit (Platelia™, Bio-Rad Laboratories, Marnes-La-Coquette, France). A virologically-confirmed episode of dengue disease is defined as a positive result in either the serotype-specific RT-PCR, or the NS1 antigen ELISA.

Active surveillance is maintained until each participant has been followed for at least 13 months after the third vaccination and until the Independent Data Monitoring Committee (IDMC) confirms that >27 cases have occurred in the per-protocol (PP) population.

All serious adverse events (SAE) are documented until the sixth month after the last vaccination, and thereafter any fatal SAE or vaccine-related SAE.

Dengue immune responses are assessed in the first 300 enrolled children at RRH in sera collected at enrolment and 28 days after each injection. Sera are also prospectively collected from all participants on Day 28 after the third injection to assess immune responses in children with virologically-confirmed dengue occurring from this timepoint. Sera are sent to GCI for measurement of serotype-specific neutralizing antibody titres against the CYD parental dengue viruses using the plaque-reduction neutralization test (PRNT₅₀) as described herein. The assay's quantitation limit is 10 (1/dil). Samples below this value are assigned the titre 5 and considered seronegative.

### Statistical Analysis

The primary objective is to determine vaccine efficacy (VE) against cases of symptomatic, virologically-confirmed dengue occurring more than 28 days after the third vaccination among children enrolled and vaccinated as planned, according to the equation: VE=100*(1−ID_{CYD}/ID_{control}), where ID is the incidence density (i.e., the number of children with virologically-confirmed dengue divided by the number of person-years at risk) in each group. With an assumed disease incidence of 1.3%, a true VE of 70%, a minimum follow-up period of 1 year after the third vaccination, and a per protocol (PP) subject attrition rate of 7.5%/year, 4002 subjects assigned with a 2:1 ratio to dengue vaccine or control are needed to demonstrate, with more than 82% power, and 95% confidence, that VE is not nul. Analyses are based on the two-sided 95% confidence interval (CI) of VE, calculated using the Exact method (Breslow N E, Day N E. Statistical Methods in Cancer Research, Volume II—The Design and Analysis of Cohort Studies. International Agency for Research on Cancer (IARC scientific publication No. 82), Lyon, France). The primary analysis is performed on the PP population, i.e. those who satisfy the enrolment criteria, who correctly receive all three doses of the assigned vaccine at Months 0, 6 (±15 days), and 12 (±30 days), and for whom group allocation is not unmasked. This analysis is repeated on the full analysis set for efficacy, in those who receive three injections. As a secondary objective, VE against dengue is determined before completion of the 3-dose vaccination regimen. In an analysis defined after unblinding, VE against each serotype individually is investigated. Analyses for safety and immunogenicity endpoints are descriptive, using 95% Cl.

### Results

Of the 4002 children enrolled, 95.9% complete the vaccinations and 91.8% are included in the per protocol (PP) analysis set for efficacy. Vaccine and control groups are comparable for age and gender. More than 90% of those sampled at baseline are positive for antibodies against dengue or JEV.

### Efficacy

During the study, 131 dengue cases (131 children had 136 episodes) are virologically-confirmed. Of these, 77 occur more than 28 days after the third injection in the PP population and are included in the primary analysis: 45 cases occurred during 2522 person-years at risk in the vaccine group, while 32 cases occurred during 1251 person-years at risk in the control group. The corresponding vaccine efficacy is 30.2% (95% Cl: −13.4-56.6). This finding is confirmed in the full analysis set (see Table 1 below). Efficacy after at least one injection is 33.4% (95% Cl: 4.1-53.5) and after at least two injections is 35.3% (95% Cl: 3.3-56.5).

TABLE 1
Serotype-specific and overall efficacy of CYD tetravalent dengue vaccine
against virologically-confirmed dengue disease

| Dengue vaccine | Control |
|---|---|
| Person- | Person- |

| | years at | Cases or | years at | Cases or | Efficacy | |
|---|---|---|---|---|---|---|
| | risk | Episodes* | risk | Episodes* | % | (95% CI) |
| >28 days after 3 injections | | | | | | |
| (per-protocol analysis) | | | | | | |
| Cases | 2522 | 45 | 1251 | 32 | 30 · 2 | (−13 · 4 − 56 · 6) |
| Serotype 1 episodes | 2536 | 9 | 1251 | 10 | 55 · 6 | (−21 · 6 − 84 · 0) |
| Serotype 2 episodes | 2510 | 31 | 1250 | 17 | 9 · 2 | (−75 · 3 − 51 · 3) |
| Serotype 3 episodes | 2541 | 1 | 1257 | 2 | 75 · 3 | (−375 · 0 − 99 · 6) |
| Serotype 4 episodes | 2542 | 0 | 1263 | 4 | 100 | (24 · 8 − 100) |
| NS1 Antigen positive | 2542 | 4 | 1265 | 0 | ND | ND |
| only episodes | | | | | | |
| >28 days after 3 injections | | | | | | |
| (Full analysis set) | | | | | | |
| Cases | 2620 | 46 | 1307 | 34 | 32 · 5 | (−8 · 5 − 57 · 6) |
| Serotype 1 episodes | 2633 | 9 | 1308 | 10 | 55 · 3 | (−22 · 5 − 83 · 9) |
| Serotype 2 episodes | 2608 | 32 | 1307 | 19 | 15 · 6 | (−57 · 6 − 53 · 6) |
| Serotype 3 episodes | 2638 | 1 | 1312 | 2 | 75 · 1 | (−378 − 99 · 6) |
| Serotype 4 episodes | 2641 | 0 | 1320 | 4 | 100 | (−24 · 3 − 100) |
| NS1 Antigen positive | 2640 | 4 | 1322 | 0 | ND | ND |
| only episodes | | | | | | |
| >28 days after at least 1 | | | | | | |
| injection (Full analysis set) | | | | | | |
| Cases | 5089 | 75 | 2532 | 56 | 33 · 4 | (4 · 1 − 53 · 5) |
| Serotype 1 episodes | 5139 | 14 | 2564 | 18 | 61 · 2 | (17 · 4 − 82 · 1) |
| Serotype 2 episodes | 5107 | 51 | 2560 | 26 | 1 · 7 | (−64 · 3 − 39 · 8) |
| Serotype 3 episodes | 5144 | 4 | 2565 | 10 | 80 · 1 | (30 · 9 − 95 · 4) |
| Serotype 4 episodes | 5149 | 1 | 2577 | 5 | 90 · 0 | (10 · 5 − 99 · 8) |
| NS1 Antigen positive | 5147 | 5 | 2579 | 1 | −150 · 5 | (−11750 − 72 · 0) |
| only episodes | 5 | | | | | |

Data are number except where indicated. ND: not determined. *A ‘case’ was defined as a first episode of dengue fever virologically-confirmed by either serotype-specific PCRs, or NS1 antigen ELISA. Serotype-specific efficacy was calculated including all episodes of that serotype; 5 children with two virologically confirmed dengue episodes during the study were therefore included twice in the serotype-specific analysis.

Post-hoc analyses reveal differing efficacy by serotype (see Table 1). Efficacy against DENV1, DENV3, and DENV4 after at least one injection is in the range 61.2%-90.0%, compared with 1.7% against DENV2. Efficacy against DENV1, DENV3, and DENV4 after three injections is in the range 55.3%-100%, compared with 15.6% against DENV2.

In those subjects that acquired virologically-confirmed dengue, a statistically significant reduction in the annual incidence rate of hospitalization was observed in the vaccinated group when compared with the control group. The relative risk (RR) after three doses was 0.523 (see Table 2).

TABLE 2
Incidence of hospitalized virologically-confirmed dengue during the trial

| | CYD Dengue Vaccine Group | | | | Control Group | | | | | |
|---|---|---|---|---|---|---|---|---|---|---|
| | (N = 2666) | | | | (N = 1331) | | | | | |
| | | | Annual | | | | Annual | | | |
| | | | Incidence | | | | Incidence | | | |
| Time | | | Rate | n | | | Rate | n | Relative Risk | |
| period | M | Cases | (95% CI) | Occurrences | M | Cases | (95% CI) | Occurrences | RR | (95% CI) |
| Year 1 | 2666 | 8 | 0. 3 | 8 | 1331 | 7 | 0.5 | 7 | 0.571 | (0.181, |
| | | | (0.1; 0.6) | | | | (0.2; 1.1) | | | 1.85) |
| Year 2 | 2557 | 24 | 0. 9 | 24 | 1282 | 23 | 1.7 | 23 | 0.523 | (0.283, |
| | | | (0.5; 1.3) | | | | (1.0; 2.5) | | | 0.970) |

Year 1 = D0 to injection 3; Year 2 = Injection 3 to the end of Active Phase

TABLE 3
Rate of hospitalisation by serotype

| | Vaccinee Group (%) | Control Group (%) |
|---|---|---|
| Serotype 1 | 8/14 (57.1) | 9/18 (50%) |
| Serotype 2 | 20/52 (38.5) | 15/27 (55.6) |
| Serotype 3 | 1/4 (25) | 3/11 (27.3) |
| Serotype 4 | 0/1 | 2/5 (40) |
| No serotype | 3/5 (60) | 1/1 (100) |
| NS1 +ve | | |
| Total | 32/76 (42.1) | 30/62 (48.4) |

### Immunogenicity

Geometric mean titres (GMT) of neutralising antibodies against dengue serotypes 1-4 on Day 28 after the third injection in the per-protocol analysis set are, respectively, 146 (95% Cl: 98.5-217), 310 (224-431), 405 (307-534), and 155 (123-196) in the vaccine group. In the control group these values are 23.9 (14.0-40.9), 52.2 (26.8-102), 48.9 (25.5-93.9), and 19.4 (11.6-32.2). Post one year GMTs are respectively 76.5; 122; 94 and 153 for serotypes 1, 2, 3 and 4.

### Safety

There are 584 SAEs during this phase of the study: 366 are reported by 11.8% (315/2666) of participants in the vaccine group, and 218 are reported by 13.2% (176/1331) of participants in the control group. There are no vaccine-related SAEs in the dengue group and there is one in the control group. SAEs observed are medical conditions consistent with the age group and showed no clustering within the 7- or 28-day post-vaccination periods.

Virologically-confirmed dengue cases occurring as a breakthrough in vaccinees were not more serious than those cases occurring in the control group.

Sequence of the prM-E Region of Circulating Wild Type Serotype 2 Strain in the Trial

The nucleotide and amino acid sequence of the prM-E region of the wild type serotype 2 strain that causes the DEN-2 cases in the trial is determined. These are set out below as SEQ ID NO: 1 and SEQ ID NO: 2 respectively. The E and the M amino acid sequences of the serotype 2 strain that causes the DEN-2 cases in the trial are described in SEQ ID NOs: 18 and 23 respectively.

>nucleotide sequence

(SEQ ID NO: 1)

ttccatctaaccacacgcaacggagaaccacacatgatcgtcggtataca
ggagaaagggaaaagtcttctgttcaaaacagaggatggtgtgaacatgt
gcaccctcatggctatggaccttggtgaattgtgtgaagacacaatcacg
tacaagtgtcctcttctcaggcagaatgagccagaagacatagactgttg
gtgcaactccacgtccacgtgggtaacctatgggacctgtaccactacgg
gagaacataggagagaaaaaagatcagtggcactcgttccacatgtggga
atgggactggagacgcgaaccgaaacatggatgtcatcagaaggggcttg
gaaacatgcccagagaattgaaacttggatcctgagacatccaggcttca
ccataatggcagcaatcctggcatacaccataggaacgacacatttccag
agagtcctgattttcatcctactgacagctgtcgctccttcaatgacaat
gcgttgcataggaatatcaaatagagactttgtagaaggggtttcaggag
gaagttgggttgacatagtcttagaacatggaagctgtgtgacgacgatg
gcaaaaaacaaaccaacattggatttcgaactgataaaaacggaagccaa
acagcctgccaccctaaggaagtactgcatagaagcaaaactaaccaaca
caacaacagaatcccgttgcccaacacaaggggaacccagcctaaaagaa
gagcaggacaagaggttcgtctgcaaacactccatggtagacagaggatg
gggaaatggatgtggattatttggaaagggaggcattgtgacctgtgcta
tgttcacatgcaaaaagaacatggaagggaaaatcgtgcaaccagaaaac
ttggaatacaccattgtggtaacacctcactcaggggaagagcatgcggt
cggaaatgacacaggaaaacacggcaaggaaatcaaagtaacaccacaga
gttccatcacagaagcagaactgacaggttatggcaccgtcacgatggag
tgctccccgagaacaggcctcgacttcaatgagatggtgttgctgcagat
ggaaaataaagcttggctggtgcataggcaatggtttctagacctgccat
taccatggctgcccggagcggataaacaagaatcaaattggatacagaaa
gaaacattggtcactttcaaaaatccccatgcgaagaaacaggatgttgt
tgttttaggatcccaagaaggggccatgcatacagcactcacaggagcca
cagaaatccaaatgtcgtcaggaaacttgctcttcactggacatctcaag
tgcaggctgagaatggacaagctacagcttaaaggaatgtcatactctat
gtgcacaggaaagtttaaagttgtgaaggaaatagcagaaacacaacatg
gaacgatagttatcagagtgcaatatgaaggggacggctctccatgtaaa
attccttttgagataatggatttggaaaaaagatatgtcttaggccgcct
gatcacagtcaacccaattgtaacagaaaaagacagcccagtcaacatag
aagcagaacctccattcggagacagttacatcatcataggagtagagccg
ggacaactgaagctcaactggttcaagaaaggaagttctatcggccaaat
gtttgagacaacgatgagaggggcgaagagaatggccattttgggtgaca
cagcctgggacttcggatccctgggaggagtgtttacatctataggaaaa
gctctccaccaagtctttggagcgatctatggggctgccttcagtggggt
ttcatggaccatgaaaatcctcataggagtcattatcacatggataggaa
tgaactcacgcagcacctcactgtctgtgtcactggtactggtgggaatt
gtgacactgtatttaggagtcatggtgcaggcc
>amino acid sequence

(SEQ ID NO: 2)

FHLTTRNGEPHMIVGIQEKGKSLLFKTEDGVNMCTLMAMDLGELCEDTIT
YKCPLLRQNEPEDIDCWCNSTSTWVTYGTCTTTGEHRREKRSVALVPHVG
MGLETRTETWMSSEGAWKHAQRIETWILRHPGFTIMAAILAYTIGTTHFQ
RVLIFILLTAVAPSMTMRCIGISNRDFVEGVSGGSWVDIVLEHGSCVTTM
AKNKPTLDFELIKTEAKQPATLRKYCIEAKLTNTTTESRCPTQGEPSLKE
EQDKRFVCKHSMVDRGWGNGCGLFGKGGIVTCAMFTCKKNMEGKIVQPEN
LEYTIVVTPHSGEEHAVGNDTGKHGKEIKVTPQSSITEAELTGYGTVTME
CSPRTGLDFNEMVLLQMENKAWLVHRQWFLDLPLPWLPGADKQESNWIQK
ETLVTFKNPHAKKQDVVVLGSQEGAMHTALTGATEIQMSSGNLLFTGHLK
CRLRMDKLQLKGMSYSMCTGKFKVVKEIAETQHGTIVIRVQYEGDGSPCK
IPFEIMDLEKRYVLGRLITVNPIVTEKDSPVNIEAEPPFGDSYIIIGVEP
GQLKLNWFKKGSSIGQMFETTMRGAKRMAILGDTAWDFGSLGGVFTSIGK
ALHQVFGAIYGAAFSGVSWTMKILIGVIITWIGMNSRSTSLSVSLVLVGI
VTLYLGVMVQA
>amino acid sequence

(SEQ ID NO: 18)

MRCIGISNRDFVEGVSGGSWVDIVLEHGSCVTTMAKNKPTLDFELIKTEA
KQPATLRKYCIEAKLTNTTTESRCPTQGEPSLKEEQDKRFVCKHSMVDRG
WGNGCGLFGKGGIVTCAMFTCKKNMEGKIVQPENLEYTIVVTPHSGEEHA
VGNDTGKHGKEIKVTPQSSITEAELTGYGTVTMECSPRTGLDFNEMVLLQ
MENKAWLVHRQWFLDLPLPWLPGADKQESNWIQKETLVTFKNPHAKKQDV
VVLGSQEGAMHTALTGATEIQMSSGNLLFTGHLKCRLRMDKLQLKGMSYS
MCTGKFKVVKEIAETQHGTIVIRVQYEGDGSPCKIPFEIMDLEKRYVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGIVTLYLGVMVQA
>amino acid sequence

(SEQ ID NO: 23)

SVALVPHVGMGLETRTETWMSSEGAWKHAQRIETWILRHPGFTIMAAILA
YTIGTTHFQRVLIFILLTAVAPSMT

### Discussion

The main finding from this study is that a safe, efficacious vaccine against dengue based on the chimeric CYD viruses is possible. Estimated efficacy against DENV1, 3 and 4 is in a range consistent with the 70% hypothesis and is statistically significant after at least one vaccination. Efficacy in a range consistent with the 70% hypothesis is not observed against DENV2. Since DENV2 is the prevalent serotype in this study, overall vaccine efficacy is diminished in this setting.

The vaccine's safety and reactogenicity profile is good, and no vaccine-related SAEs and no safety signals are identified during the review of AEs and SAEs collected from over two years of active follow-up of more than 2600 vaccinees. Theoretical safety concerns associated with the potential enhancement of the rate or severity of dengue disease by an incomplete immune response against the four serotypes of dengue have previously hampered vaccine development. In this trial, the absence of disease enhancement in the presence of an incomplete immune response against the circulating DENV2 viruses is an important and reassuring finding. For instance, cases in vaccinees do not differ from cases in controls in terms of factors such as the duration of fever or in terms of the classical clinical signs of dengue such as bleeding, plasma leakage or thrombocytopenia. Furthermore, severe dengue was not more frequent among vaccinees than controls at any point during the trial).

It was also demonstrated that, in those subjects that acquired virologically-confirmed dengue, a statistically significant reduction in the annual incidence rate of hospitalization was observed in the vaccinated group when compared with the control group. This reduction was seen in those subjects that acquired virologically-confirmed dengue of serotype 2 (see Table 3).

The results observed in respect of DENV2 may be explained by a number of contributing factors. For instance, there is a possible antigenic mismatch between the CYD2 vaccine virus and the DENV2 virus that causes disease in the trial. In the 1990s, the Asian 1 genotype of DENV2 emerged in South-East Asia, replacing the previously dominant Asian/American lineage of viruses. Several mutations identified in Domain 2 of the E protein (E83, and in particular E226 and E228) are suggestive of changing viral fitness and antigenicity. The donor wild-type virus for the CYD2 vaccine (and the challenge strain used in the PRNT50) was a clinical isolate from Bangkok in 1980 (Guirakhoo F et al., J Virol 2000, 74: 5477-85). While this virus is also classified as belonging to the Asian I genotype, the above-mentioned key amino acid residues in this virus (and thus in CYD2) correspond to those of the Asian/American genotype (Hang et al PLoS Negl Trop Dis. 2010 Jul. 20; 4(7):e757).

Additionally, there are two extremely rare mutations in the prM-E sequence of the CYD2 vaccine that may also contribute to a mismatched immune response. These mutations are at positions prM24 and E251 (Guirakhoo et al, J. Virol. (2004) 78 (9): 4761).

The results observed against DENV2 are not associated with an absence of immunogenicity in the PRNT₅₀ assay. Neutralising antibody responses after vaccination against DENV2 are higher than those against DENV1 and DENV3.

In conclusion, the present study constitutes the first ever demonstration that a safe and efficacious dengue vaccine is possible and represents a major milestone in dengue vaccine development.

### Example 2

### Identification of Optimized Dengue Vaccinal Strains of Serotype 2

The objective of the present example is to identify dengue virus strains of serotype 2 which provide the basis for generating optimized dengue vaccine compositions against dengue virus of serotype 2, wherein said optimized dengue vaccine compositions provide improved efficacy in comparison to Chimerivax™ CYD-2 when used in a method according to the present invention.

Criteria determining the selection of optimized strains for the determination of a universal dengue 2 antigen include: (i) recently circulating strain; (ii) balanced selection between Asian and American strains; (iii) an optimized strain should have a prM-E sequence that is as similar as possible to a calculated global consensus sequence generated by aligning the available prM-E sequences of dengue viruses of serotype 2; (iv) amino acid variations that are predicted to impact antibody recognition should be avoided; (v) rare amino acids at a particular positions in the prM and E sequences should be avoided, especially in the E protein ectodomain (a rare amino acid at a particular position is defined as a amino acid that appears at that position in less than 15% of the aligned sequences); (vi) optimized strains for which some previous laboratory experience exists are preferred and (vii) a dengue antigen that leads to a balanced immune response in a tetravalent composition.

Criteria determining the selection of optimized strains for a local dengue 2 antigen (i.e. that is especially effective against a wild type dengue virus circulating in a particular area) are criteria (i) and (vii).

### Methods

### Databases

Sequence are retrieved from the National Center for Biotechnology Information (NCBI) Dengue virus variation database (www.ncbi.nlm.nih.gov/genomes/VirusVariation/Database/nph-select.cgi?tax_id=12637).

### Sequence Analyses

Sequence alignments are performed using the MUSCLE algorithm (Edgar, R. C. (2004) MUSCLE: multiple sequence alignment with high accuracy and high throughput. Nucleic Acids Res, 32(5):1792-1797).

Sequence alignment outputs are generated in Vector NTi version 9, module AlignX (Invitrogen). Sequence similarity searches are carried out using the BLAST algorithm (Altschul, S. F., Gish, W., Miller, W., Myers, E. W., and Lipman, D. J. (1990) Basic local alignment search tool. J Mol Biol, 215(3):403-410).

Sequence Numbering for prM-E Sequences

The sub-sequences included in the prM-E sequences may be numbered in various ways: (i) the total prM-E protein sequence is numbered from position 1 to position 661, with the preM protein sequence designated as position 1 to position 90/91, the M protein sequence designated as position 91/92 to position 166 and the E protein sequence designated as position 167 to position 661; (ii) the prM and M protein sequences are numbered together, i.e. from position 1 to position 166 of the total sequence and E is numbered separately from position 1 to position 495; (iii) the prM, M and E sequences are numbered separately, i.e. prM is numbered from position 1 to 90/91, M is numbered from 1 to 75/76 and E from position 1 to position 495.

### Results

### Public Sequences Retrieval

All available dengue virus serotype 2 full length prM and E protein sequences are downloaded from the NCBI Dengue database. Download of sequences takes place on two separate occasions—on 4 Oct. 2010 and in 2011. On the first occasion 669 sequences are downloaded and on the second occasion approximately 3200 sequences are downloaded.

### Global Consensus Sequence Generation

On each occasion, all retrieved protein sequences are aligned to generate a global consensus sequence for the prM and E proteins of dengue virus of serotype 2. By definition, the global consensus sequence is an artificial sequence containing the most frequently encountered amino acid at each position. The global consensus sequences for the 2010 alignment and the 2011 alignment only differ by two amino acids. In the 2010 alignment, the global consensus sequence contains isoleucine and valine at positions 129 and 308 respectively of the E protein (by reference to the 1-495 E sequence numbering) and, by contrast, in the 2011 alignment, the global consensus sequence contains valine and isoleucine at positions 129 and 308 respectively of the E protein (by reference to the 1-495 E sequence numbering). The differences in the 2010 and 2011 global consensus sequences is explained by the fact that the respective percentages of strains containing valine or isoleucine at those positions is close to 50%. The global consensus sequence for the prM-E sequence is therefore represented as follows:

(SEQ ID NO: 3)

fhlttrngephmivgrqekgksllfktedgvnmctlmaidlgelcedtit
ykcpllrqnepedidcwcnststwvtygtctttgehrrekrsvalvphvg
mgletrtetwmssegawkhvqrietwilrhpgftimaailaytigtthfq
ralifilltavapsmtMRCIGISNRDFVEGVSGGSWVDIVLEHGSCVTTM
AKNKPTLDFELIKTEAKQPATLRKYCIEAKLTNTTTESRCPTQGEPSLNE
EQDKRFVCKHSMVDRGWGNGCGLFGKGGIVTCAMFTCKKNMEGKXVQPEN
LEYTIVITPHSGEEHAVGNDTGKHGKEIKITPQSSITEAELTGYGTVTME
CSPRTGLDFNEMVLLQMEDKAWLVHRQWFLDLPLPWLPGADTQGSNWIQK
ETLVTFKNPHAKKQDVVVLGSQEGAMHTALTGATEIQMSSGNLLFTGHLK
CRLRMDKLQLKGMSYSMCTGKFKZVKEIAETQHGTIVIRVQYEGDGSPCK
IPFEIMDLEKRHVLGRLITVNPIVTEKDSPVNIEAEPPFGDSYIIIGVEP
GQLKLNWFKKGSSIGQMFETTMRGAKRMAILGDTAWDFGSLGGVFTSIGK
ALHQVFGAIYGAAFSGVSWTMKILIGVIITWIGMNSRSTSLSVSLVLVGV
VTLYLGVMVQA

The global consensus sequence for the E sequence is represented as follows:

(SEQ ID NO: 12)

MRCIGISNRDFVEGVSGGSWVDIVLEHGSCVTTMAKNKPTLDFELIKTEA
KQPATLRKYCIEAKLTNTTTESRCPTQGEPSLNEEQDKRFVCKHSMVDRG
WGNGCGLFGKGGIVTCAMFTCKKNMEGKXVQPENLEYTIVITPHSGEEHA
VGNDTGKHGKEIKITPQSSITEAELTGYGTVTMECSPRTGLDFNEMVLLQ
MEDKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKZVKEIAETQHGTIVIRVQYEGDGSPCKIPFEIMDLEKRHVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGVVTLYLGVMVQA

In the above sequence, the global consensus prM sequence is shown in lower case letters and the E sequence is shown in upper case letters. The amino acid positions denoted as X (position 129 of the E sequence) and Z (position 308 of the E sequence) are each independently Val or Ile, i.e. the proportion of aligned amino acid sequences including Val or Ile at those positions is close to 50%.

### Determination of Minor Amino Acid Residues and Analysis of the Chimerivax™ CYD2 Sequence

A list of variable amino acid positions is established from the global alignment containing all amino acid positions varying in at least 5% of the aligned sequences. In addition, any amino acid from the sequence of the prM and E proteins of Chimerivax™ CYD2 that do not match the global consensus sequence are also identified. The results are shown in Table 4 (N.B., in the table, the prM and M protein sequences are numbered together, i.e. from position 1 to position 166 of the total sequence and E is numbered separately from position 1 to position 495).

TABLE 4
Dengue virus serotype 2 variable residues and CYD2 comparison

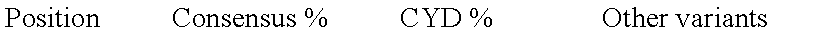

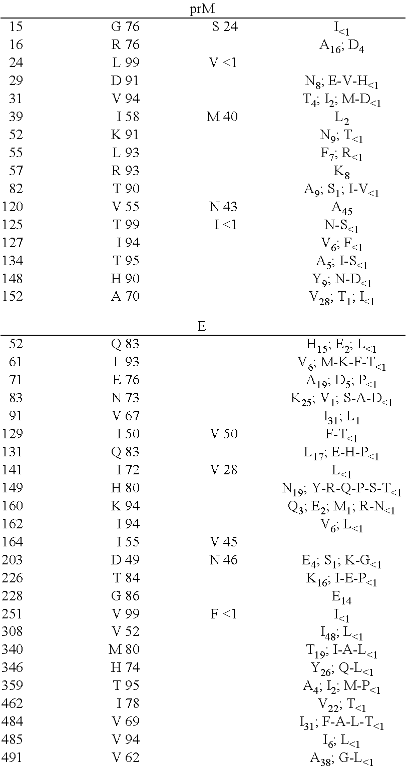

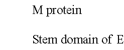

A total of 41 amino acid positions are identified in the prM and E sequences which either vary from the global consensus sequence in at least 5% of the aligned sequences and/or differ from the sequence of the prM and E proteins in CYD2. Ten amino acid positions in the sequence of the prM and E proteins in CYD2 differ from the global consensus sequence (5 positions in E, 2 positions in M and 3 in its precursor part, see Table 4). Five out of the ten differing residues present a variation distribution close to 50:50, suggesting a naturally variable position. Only three positions in the CYD2 prM-E sequence appear as very minor variants (pr-24 Val, M-125 Ile and E-251 Phe).

### Impact Analysis of Variations in the E and M Proteins

To gain further insight into the variable positions, changes in the E protein ectodomain (amino acids 1-395), the most important domain for the seroneutralisation by the immune system are further analysed.

Using information available from a published 3D structure of the soluble ectodomain of the E protein of a dengue virus of serotype 2 (Modis, Y., et al. (2003) Proc Natl Acad Sci USA, 100(12):6986-6991), a 3D model of the Dengue virus particle surface is reconstructed. This allows a fine tuned assessment of the accessibility of each amino acid from the E ectodomain, which in turn is used in association with the variability level and the nature of the amino acid change to assess a potential impact of CYD2 variations on antibody recognition.

The analysis demonstrates that two variations in the Chimerivax™ CYD2 sequence from the global consensus sequence (Val 141 and Val 164 of the E protein) are completely buried in the 3D structure and so cannot directly interact with an antibody at the surface of the virion. Position 129 of the E protein is a 50:50 variable amino acid position between Val (Chimerivax™ CYD2) and Ile (global consensus sequence) and the substitution is also a fully conservative change. The potential impact of these variations is therefore considered as very limited.

The variation at position 203 of the E protein (Asn in Chimerivax™ CYD2 and Asp in the global consensus sequence) could potentially have an impact (well exposed residue, change of charge) but the distribution of the variation among strains is close to 50:50, suggesting a naturally variable position.

The variation at position 251 of the E protein of Chimerivax™ CYD2 (Phe in Chimerivax™ CYD2 and Val in the global consensus sequence) is extremely rare among retrieved strains. Such a variation could have some impact on recognition by an antibody, as it is rare, rather well exposed at the surface of the virion (29%) and corresponds to a non-conservative amino acid change.

The modeling analysis described above identifies two other position variations in the E protein that could have a potential impact on antibody recognition (positions 226 and 228), although Chimerivax™ CYD2 does not vary from the global consensus sequence at those positions. Therefore in identifying optimised serotype 2 strains, variations from the global consensus sequence at those positions (i.e. Thr at position 226 and glycine at position 228) are preferably avoided for a universal dengue 2 vaccine.

Without being bound by theory, the present inventors consider that the impact of amino acid variations in dengue virus sequences can also be assessed using a scoring method which takes into account a number of relevant factors. In particular this method takes into account the genome location of the variation (G), the nature of the amino acid change (B), 3D mapping (M) and known variants at the position in question (DB), wherein the score is calculated as G×B×M×DB. A score of 0 would be classified as no expected impact, a score of >0 to 10 would be classified as a low expected impact, a score of >10 to 25 would be classified as a median expected impact and a score of >25 would be classified as a high expected impact.

The genome location (G) score is 0 if the amino acid is located in the M part of the prM/M protein (i.e. position 92 to 166 of the prM/M sequence) or in position 396 to 495 of the E protein. The genome location score is 1 if the amino acid is located in prM part of the prM/M protein (i.e. position 1 to 91 of the prM/M sequence) or in position 1 to 395 of the E protein.

The score related to the nature of the amino acid change (B) is calculated as B=100−[(Blosum95 score+6)×10], wherein the Blosum95 score for different amino acid substitutions is as shown in Table 5 below.

TABLE 5

| | A | R | N | D | C | Q | E | G | H | I | L | K | M | F | P | S | T | W | Y | V | B | Z | X | * |
|---|---|---|---|---|---|---|---|---|---|---|---|---|---|---|---|---|---|---|---|---|---|---|---|---|
| A | | −2 | −2 | −3 | −1 | −1 | −1 | −1 | −3 | −2 | −2 | −1 | −2 | −3 | −1 | 1 | 0 | −4 | −3 | −1 | −3 | −1 | −1 | −6 |
| R | −2 | | −1 | −3 | −5 | 0 | −1 | −4 | −1 | −4 | −3 | 2 | −2 | −4 | −3 | −2 | −2 | −4 | −3 | −4 | −2 | −1 | −2 | −6 |
| N | −2 | −1 | | 1 | −4 | 0 | −1 | −1 | 0 | −4 | −5 | 0 | −3 | −4 | −3 | 0 | −1 | −5 | −3 | −4 | 4 | −1 | −2 | −6 |
| D | −3 | −3 | 1 | | −5 | −1 | 1 | −2 | −2 | −5 | −5 | −2 | −5 | −5 | −3 | −1 | −2 | −6 | −5 | −5 | 4 | 0 | −2 | −6 |
| C | −1 | −5 | −4 | −5 | | −4 | −6 | −5 | −5 | −2 | −3 | −5 | −3 | −3 | −5 | −2 | −2 | −4 | −4 | −2 | −4 | −5 | −3 | −6 |
| Q | −1 | 0 | 0 | −1 | −4 | | 2 | −3 | 1 | −4 | −3 | 1 | −1 | −4 | −2 | −1 | −1 | −3 | −3 | −3 | −1 | 4 | −1 | −6 |
| E | −1 | −1 | −1 | 1 | −6 | 2 | | −3 | −1 | −4 | −4 | 0 | −3 | −5 | −2 | −1 | −2 | −5 | −4 | −3 | 0 | 4 | −2 | −6 |
| G | −1 | −4 | −1 | −2 | −5 | −3 | −3 | | −3 | −6 | −5 | −3 | −4 | −5 | −4 | −1 | −3 | −5 | −5 | −5 | −2 | −3 | −3 | −6 |
| H | −3 | −1 | 0 | −2 | −5 | 1 | −1 | −3 | | −4 | −4 | −1 | −3 | −2 | −3 | −2 | −2 | −3 | 1 | −4 | −1 | 0 | −2 | −6 |
| I | −2 | −4 | −4 | −5 | −2 | −4 | −4 | −6 | −4 | | 1 | −4 | 1 | −1 | −4 | −3 | −2 | −4 | −2 | 3 | −5 | −4 | −2 | −6 |
| L | −2 | −3 | −5 | −5 | −3 | −3 | −4 | −5 | −4 | 1 | | −3 | 2 | 0 | −4 | −3 | −2 | −3 | −2 | 0 | −5 | −4 | −2 | −6 |
| K | −1 | 2 | 0 | −2 | −5 | 1 | 0 | −3 | −1 | −4 | −3 | | −2 | −4 | −2 | −1 | −1 | −5 | −3 | −3 | −1 | 0 | −1 | −6 |
| M | −2 | −2 | −3 | −5 | −3 | −1 | −3 | −4 | −3 | 1 | 2 | −2 | | −1 | −3 | −3 | −1 | −2 | −3 | 0 | −4 | −2 | −2 | −6 |
| F | −3 | −4 | −4 | −5 | −3 | −4 | −5 | −5 | −2 | −1 | 0 | −4 | −1 | | −5 | −3 | −3 | 0 | 3 | −2 | −5 | −4 | −2 | −6 |
| P | −1 | −3 | −3 | −3 | −5 | −2 | −2 | −4 | −3 | −4 | −4 | −2 | −3 | −5 | | −2 | −2 | −5 | −5 | −4 | −3 | −2 | −3 | −6 |
| S | 1 | −2 | 0 | −1 | −2 | −1 | −1 | −1 | −2 | −3 | −3 | −1 | −3 | −3 | −2 | | 1 | −4 | −3 | −3 | −1 | −1 | −1 | −6 |
| T | 0 | −2 | −1 | −2 | −2 | −1 | −2 | −3 | −2 | −2 | −2 | −1 | −1 | −3 | −2 | 1 | | −4 | −3 | −1 | −1 | −2 | −1 | −6 |
| W | −4 | −4 | −5 | −6 | −4 | −3 | −5 | −5 | −3 | −4 | −3 | −5 | −2 | 0 | −5 | −4 | −4 | | 2 | −3 | −6 | −4 | −4 | −6 |
| Y | −3 | −3 | −3 | −5 | −4 | −3 | −4 | −5 | 1 | −2 | −2 | −3 | −3 | 3 | −5 | −3 | −3 | 2 | | −3 | −4 | −4 | −2 | −6 |
| V | −1 | −4 | −4 | −5 | −2 | −3 | −3 | −5 | −4 | 3 | 0 | −3 | 0 | −2 | −4 | −3 | −1 | −3 | −3 | | −5 | −3 | −2 | −6 |
| B | −3 | −2 | 4 | 4 | −4 | −1 | 0 | −2 | −1 | −5 | −5 | −1 | −4 | −5 | −3 | −1 | −1 | −6 | −4 | −5 | | 0 | −2 | −6 |
| Z | −1 | −1 | −1 | 0 | −5 | 4 | 4 | −3 | 0 | −4 | −4 | 0 | −2 | −4 | −2 | −1 | −2 | −4 | −4 | −3 | 0 | | −1 | −6 |
| X | −1 | −2 | −2 | −2 | −3 | −1 | −2 | −3 | −2 | −2 | −2 | −1 | −2 | −2 | −3 | −1 | −1 | −4 | −2 | −2 | −2 | −1 | | −6 |
| * | −6 | −6 | −6 | −6 | −6 | −6 | −6 | −6 | −6 | −6 | −6 | −6 | −6 | −6 | −6 | −6 | −6 | −6 | −6 | −6 | −6 | −6 | −6 | |

B = Asx, Z = Glx, X = Any and * = Stop

The M value depends on whether the amino acid is or is not located at the prM/E interface. For example, for CYD2 as used in Example 1, the amino acids that are located at the interface are prM residues 6, 7, 39, 40, 46-54, 56, 59-65, 67, 74 and 77 and E residues 64-72, 82-84, 101-104, 106-108 and 244-247. Where an amino acid is located at the interface, M equals 1. Where an amino acid is not located at the interface, M=Y×SAS %. Y is 1 if the amino acid is located in an “up” position (i.e. directed towards the external environment); Y is 0.5 if the amino acid is located on the “side” of the molecule (i.e. the amino acid is neither directed towards the external environment nor towards the capsid) and Y is 0 if the amino acid is located in a “down” position (i.e. directed towards the capsid). The solvent accessibility surface % (SAS %) value is generated using the Discovery Studio 3D modeling software (Accelrys, Inc., CA, USA).

The DB value is 0 when the amino acid substitution results in an amino acid at the substitution position which is the most common amino acid at that position in the dengue sequences present in the GenBank database (http://www.ncbi.nlm.nih.gov). The DB value is 0.25 when the amino acid substitution results in an amino acid at the substitution position which is found in more than 5% of the dengue sequences present in the database (but is not the most common amino acid at that position). The DB value is 0.50 when the amino acid substitution results in an amino acid at the substitution position which is found in less than 5% of the dengue sequences present in the database (except unique substitutions). The DB value is 1 when the substitution amino acid is unique.

During replication, viruses may acquire a mutation leading to an amino acid substitution. The above-mentioned method provides a means to determine the effect of such mutations on the progeny of the mutated viruses.

Preferred sequences (i.e. sequences that are considered to be satisfactorily close to the identified consensus sequence) may have: (i) at most two, preferably one or no high-impact amino acid substitutions; (ii) at most three, preferably two or one, or no median impact amino acid substitutions; and/or (iii) at most five, four, three, two or one low impact amino acid substitutions.

### Identification of Optimized Serotype 2 Strains

Optimised serotype 2 strains are identified on the basis of the selection criteria described above.

A BLAST search is conducted to identify the strain having the closest sequence to the prM-E global consensus sequence in all of the available sequences. No sequence that is 100% identical to the prM-E global consensus sequence is found, but the best hit is a sequence from strain BID-V585 (NCBI Protein ID no. ACA58343; Genome ID no. EU529706; isolated from Puerto Rico in 2006) which shows only one variation from the global consensus sequence, at position 91 (Val in the global consensus sequence and Ile in BID-V585). The BID-V585 prM-E sequence contains 13 variations from the Chimerivax™ CYD-2 prM-E sequence.

A further strain selection is made so as to provide geographical balance in strain origin. Therefore a recently isolated Asian strain showing a good score in the BLAST analysis (strain MD-1280; NCBI Protein ID no. CAR65175; Genome ID no. FM21043; isolated from Viet Nam in 2004) is selected. Despite showing 6 variations with the global consensus sequence across prM-E, 3 of the 6 variations are identified as versatile positions naturally varying in more than 30% of the strains. The MD-1280 prM-E sequence contains 15 variations from the Chimerivax™ CYD-2 prM-E sequence.

A further strain selection is made on the basis of a large amount of previously accumulated experience with the strain. It is the PDK53-16681 strain, also known as the LAV-2 strain, a live-attenuated virus derived from Dengue serotype 2 16681 strain from Mahidol University (NCBI Protein ID no. AAA73186; Genome ID no. M84728; isolated from Thailand in 1964; Blok, J., et al. (1992); Virology 187 (2), 573-590). The LAV-2 prM-E sequence contains 10 variations from the global consensus sequence and 13 variations from the Chimerivax™ CYD-2 prM-E sequence.

A further strain selected on the basis of the above-mentioned criteria is strain PR/DB023 (NCBI Protein ID no. AEN71248; Genome ID no. JF804036; isolated from Puerto Rico in 2007). The PR/DB023 prM-E sequence contains 3 variations from the global consensus sequence and 13 variations from the Chimerivax™ CYD-2 prM-E sequence.

None of the selected strains contain the rare amino acids present in the Chimerivax™ CYD-2 prM-E sequence, i.e. Val at pr-24, Ile at M-125 and Phe at E-251.

### PrM to E Nucleotide Sequences of the Four Selected Strains

>LAV-2 prME nucleotide sequence

(SEQ ID NO: 4)

ttccatttaaccacacgtaacggagaaccacacatgatcgtcagcagaca
agagaaagggaaaagtcttctgtttaaaacagaggttggcgtgaacatgt
gtaccctcatggccatggaccttggtgaattgtgtgaagacacaatcacg
tacaagtgtccccttctcaggcagaatgagccagaagacatagactgttg
gtgcaactctacgtccacgtgggtaacttatgggacgtgtaccaccatgg
gagaacatagaagagaaaaaagatcagtggcactcgttccacatgtggga
atgggactggagacacgaactgaaacatggatgtcatcagaaggggcctg
gaaacatgtccagagaattgaaacttggatcttgagacatccaggcttca
ccatgatggcagcaatcctggcatacaccataggaacgacacatttccaa
agagccctgattttcatcttactgacagctgtcactccttcaatgacaAT
GCGTTGCATAGGAATGTCAAATAGAGACTTTGTGGAAGGGGTTTCAGGAG
GAAGCTGGGTTGACATAGTCTTAGAACATGGAAGCTGTGTGACGACGATG
GCAAAAAACAAACCAACATTGGATTTTGAACTGATAAAAACAGAAGCCAA
ACAGCCTGCCACCCTAAGGAAGTACTGTATAGAGGCAAAGCTAACCAACA
CAACAACAGAATCTCGCTGCCCAACACAAGGGGAACCCAGCCTAAATGAA
GAGCAGGACAAAAGGTTCGTCTGCAAACACTCCATGGTAGACAGAGGATG
GGGAAATGGATGTGGACTATTTGGAAAGGGAGGCATTGTGACCTGTGCTA
TGTTCAGATGCAAAAAGAACATGGAAGGAAAAGTTGTGCAACCAGAAAAC
TTGGAATACACCATTGTGATAACACCTCACTCAGGGGAAGAGCATGCAGT
CGGAAATGACACAGGAAAACATGGCAAGGAAATCAAAATAACACCACAGA
GTTCCATCACAGAAGCAGAATTGACAGGTTATGGCACTGTCACAATGGAG
TGCTCTCCAAGAACGGGCCTCGACTTCAATGAGATGGTGTTGCTGCAGAT
GGAAAATAAAGCTTGGCTGGTGCACAGGCAATGGTTCCTAGACCTGCCGT
TACCATGGTTGCCCGGAGCGGACACACAAGGGTCAAATTGGATACAGAAA
GAGACATTGGTCACTTTCAAAAATCCCCATGCGAAGAAACAGGATGTTGT
TGTTTTAGGATCCCAAGAAGGGGCCATGCACACAGCACTTACAGGGGCCA
CAGAAATCCAAATGTCATCAGGAAACTTACTCTTCACAGGACATCTCAAG
TGCAGGCTGAGAATGGACAAGCTACAGCTCAAAGGAATGTCATACTCTAT
GTGCACAGGAAAGTTTAAAGTTGTGAAGGAAATAGCAGAAACACAACATG
GAACAATAGTTATCAGAGTGCAATATGAAGGGGACGGCTCTCCATGCAAG
ATCCCTTTTGAGATAATGGATTTGGAAAAAAGACATGTCTTAGGTCGCCT
GATTACAGTCAACCCAATTGTGACAGAAAAAGATAGCCCAGTCAACATAG
AAGCAGAACCTCCATTTGGAGACAGCTACATCATCATAGGAGTAGAGCCG
GGACAACTGAAGCTCAACTGGTTTAAGAAAGGAAGTTCTATCGGCCAAAT
GTTTGAGACAACAATGAGGGGGGCGAAGAGAATGGCCATTTTAGGTGACA
CAGCCTGGGATTTTGGATCCTTGGGAGGAGTGTTTACATCTATAGGAAAG
GCTCTCCACCAAGTCTTTGGAGCAATCTATGGAGCTGCCTTCAGTGGGGT
TTCATGGACTATGAAAATCCTCATAGGAGTCATTATCACATGGATAGGAA
TGAATTCACGCAGCACCTCACTGTCTGTGACACTAGTATTGGTGGGAATT
GTGACACTGTATTTGGGAGTCATGGTGCAGGCC
UPPERCASE: E coding sequence; lowercase:
prM coding sequence
> BID/V585 - prME nucleotide sequence

(SEQ ID NO: 5)

ttccatttaaccacacgtaatggagaaccacacatgatcgttggtaggca
agagaaagggaaaagtcttctgtttaaaacagaggatggtgttaacatgt
gcaccctcatggccatagaccttggtgaattgtgtgaagatacaatcacg
tacaagtgccccctcctcaggcaaaatgaaccagaagacatagattgttg
gtgcaactctacgtccacatgggtaacttatgggacatgtaccaccacag
gagaacacagaagagaaaaaagatcagtggcactcgttccacatgtgggc
atgggactggagacacgaactgaaacatggatgtcatcagaaggggcctg
gaaacatgttcagagaattgaaacctggatcttgagacatccaggcttta
ccataatggcagcaatcctggcatataccataggaacgacacatttccaa
agggctctgatcttcattttactgacagccgttgctccttcaatgacaAT
GCGTTGCATAGGAATATCAAATAGAGACTTCGTAGAAGGGGTTTCAGGAG
GAAGTTGGGTTGACATAGTCTTAGAACATGGAAGTTGTGTGACGACGATG
GCAAAAAATAAACCAACATTGGATTTTGAACTGATAAAAACAGAAGCCAA
ACAACCTGCCACTCTAAGGAAGTACTGTATAGAAGCAAAGCTGACCAATA
CAACAACAGAATCTCGTTGCCCAACACAAGGGGAACCCAGTCTAAATGAA
GAGCAGGACAAAAGGTTCATCTGCAAACACTCCATGGTAGACAGAGGATG
GGGAAATGGATGTGGATTATTTGGAAAGGGAGGCATTGTGACCTGTGCTA
TGTTCACATGCAAAAAGAACATGGAAGGAAAAGTCGTGCAGCCAGAAAAT
CTGGAATACACCATCGTGATAACACCTCACTCAGGAGAAGAGCACGCTGT
AGGTAATGACACAGGAAAGCATGGCAAGGAAATCAAAATAACACCACAGA
GCTCCATCACAGAAGCAGAACTGACAGGCTATGGCACTGTCACGATGGAG
TGCTCTCCGAGAACGGGCCTCGACTTCAATGAGATGGTACTGCTGCAGAT
GGAAGACAAAGCTTGGCTGGTGCACAGGCAATGGTTCCTAGACCTGCCGT
TACCATGGCTACCCGGAGCGGACACACAAGGATCAAATTGGATACAGAAA
GAGACGTTGGTCACTTTCAAAAATCCCCACGCGAAGAAACAGGACGTCGT
TGTTTTAGGATCTCAAGAAGGGGCCATGCACACGGCACTTACAGGGGCCA
CAGAAATCCAGATGTCATCAGGAAACTTACTGTTCACAGGACATCTCAAG
TGTAGGCTGAGAATGGACAAATTACAGCTTAAAGGAATGTCATACTCTAT
GTGTACAGGAAAGTTTAAAATTGTGAAGGAAATAGCAGAAACACAACATG
GAACAATAGTTATCAGAGTACAATATGAAGGGGACGGCTCTCCATGTAAG
ATTCCTTTTGAGATAATGGATTTGGAAAAAAGACACGTCCTAGGTCGCCT
GATTACAGTGAACCCAATCGTAACAGAAAAAGATAGCCCAGTCAACATAG
AAGCAGAACCTCCATTCGGAGACAGCTACATCATCATAGGAGTAGAGCCG
GGACAATTGAAACTCAATTGGTTCAAGAAGGGAAGTTCCATTGGCCAAAT
GTTTGAGACAACAATGAGAGGAGCGAAGAGAATGGCCATTTTAGGTGACA
CAGCCTGGGATTTTGGATCCCTGGGAGGAGTGTTTACATCTATAGGAAAG
GCTCTCCACCAAGTTTTCGGAGCAATCTATGGGGCTGCTTTTAGTGGGGT
CTCATGGACTATGAAAATCCTCATAGGAGTTATTATCACATGGATAGGAA
TGAATTCACGTAGCACCTCACTGTCTGTGTCACTAGTATTGGTGGGAGTC
GTGACACTGTACTTGGGGGTTATGGTGCAGGCT
>PR/DB023 prME nucleotide sequence

(SEQ ID NO: 6)

ttccatttaaccacacgtaatggagaaccacacatgatcgttggtaggca
agagaaagggaaaagtcttctgttcaaaacagaggatggtgttaacatgt
gtaccctcatggccatagaccttggtgaattgtgtgaagatacaatcacg
tacaagtgccccctcctcaggcaaaatgaaccagaagacatagattgttg
gtgcaactctacgtccacatgggtaacttatgggacatgtaccaccacag
gagaacacagaagagaaaaaagatcagtggcactcgttccacatgtgggc
atgggactggagacacgaactgaaacatggatgtcatcagaaggggcctg
gaaacatgttcagagaattgaaacctggatattgagacatccaggcttta
ccataatggcagcaatcctggcatataccataggaacgacacatttccaa
agggctctgatcttcattttactgacagccgtcgctccttcaatgacaAT
GCGTTGCATAGGAATATCAAATAGAGACTTCGTAGAAGGGGTTTCAGGAG
GAAGTTGGGTTGACATAGTCTTAGAACATGGAAGTTGTGTGACGACGATG
GCAAAAAATAAACCAACATTGGATTTTGAACTGATAAAAACAGAAGCCAA
ACAACCTGCCACTCTAAGGAAGTACTGTATAGAAGCAAAGCTGACCAATA
CAACAACAGAATCTCGTTGCCCAACACAAGGGGAACCCAGTCTAAATGAA
GAGCAGGACAAAAGGTTCATCTGCAAACACTCCATGGTAGACAGAGGATG
GGGAAATGGATGTGGATTATTTGGAAAAGGAGGCATTGTAACCTGTGCTA
TGTTCACATGCAAAAAGAACATGGAAGGAAAAGTTGTGCTGCCAGAAAAT
CTGGAATACACCATCGTGATAACACCTCACTCAGGAGAAGAGCACGCTGT
AGGTAATGACACAGGAAAACATGGCAAGGAAATTAAAATAACACCACAGA
GTTCCATCACAGAAGCAGAACTGACAGGCTATGGCACTGTCACGATGGAG
TGCTCTCCGAGAACGGGCCTCGACTTCAATGAGATGGTGCTGCTGCAGAT
GGAAGACAAAGCCTGGCTGGTGCACAGGCAATGGTTCCTAGATCTGCCGT
TACCATGGCTACCCGGAGCGGACACACAAGGATCAAATTGGATACAGAAA
GAGACGTTGGTCACTTTCAAAAATCCCCACGCGAAGAAACAGGACGTCGT
TGTTTTAGGATCTCAAGAAGGGGCCATGCACACGGCACTTACAGGGGCCA
CAGAAATCCAGATGTCATCAGGAAACTTACTGTTCACAGGACATCTCAAG
TGTAGGCTGAGAATGGACAAATTACAGCTTAAAGGAATGTCATACTCTAT
GTGTACAGGAAAGTTTAAAATTGTGAAGGAAATAGCAGAAACACAACATG
GAACAATAGTTATCAGAGTACAATATGAAGGGGACGGCTCTCCATGTAAG
ATTCCTTTTGAGATAATGGATTTAGAAAAAAGACACGTCCTAGGTCGCCT
GATTACAGTGAACCCAATCGTAACAGAAAAAGATAGCCCAGTCAACATAG
AAGCAGAACCTCCATTCGGAGACAGCTACATCATCATAGGAGTAGAGCCG
GGACAATTGAAACTCAATTGGTTCAAGAAGGGAAGTTCCATTGGCCAAAT
GTTTGAGACAACAATGAGAGGAGCGAAGAGAATGGCCATTTTAGGTGACA
CAGCCTGGGATTTTGGATCCCTGGGAGGAGTGTTTACATCTATAGGAAAG
GCTCTCCACCAAGTTTTCGGAGCAATCTATGGGGCTGCTTTTAGTGGGGT
CTCATGGACTATGAAAATCCTCATAGGAGTTATCATCACATGGATAGGAA
TGAATTCACGTAGCACCTCACTGTCTGTGTCACTAGTATTGGTGGGAGTC
GTGACACTGTACTTGGGGGTTATGGTGCAGGCT
>MD1280 prME nucleotide sequence

(SEQ ID NO: 7)

ttccatttaaccacacgaaatggagaaccacacatgatcgttggcagaca
agagaaagggaaaagccttctgtttaaaacagaggatggtgtgaacatgt
gtaccctcatggccattgatcttggtgaattgtgtgaagatacaatcacg
tacaagtgccccctcctcaggcagaatgaaccagaagatatagattgttg
gtgcaactccacgtccacatgggtaacttatgggacgtgtaccaccacag
gagaacacagaagagaaaaaagatcagtggcactcgttccacatgtgggt
atgggactggagacacgaactgaaacatggatgtcgtcagaaggggcctg
gaaacacgctcagagaattgaaacttggatcttgagacatccaggcttta
ccataatggcagcaatcctggcatataccgtaggaacgacacatttccaa
agggccctgattttcatcttactggcagctgtcgctccttcaatgacaAT
GCGTTGCATAGGAATATCAAATAGAGACTTTGTAGAAGGGGTTTCAGGAG
GAAGCTGGGTTGACATAGTCTTAGAACATGGAAGTTGTGTGACGACAATG
GCAAAAAATAAACCAACACTGGATTTTGAACTGATAAAAACAGAAGCCAA
ACAACCTGCCACTCTAAGGAAGTACTGTATAGAGGCAAAGCTGACCAATA
CAACAACAGAATCTCGTTGCCCAACACAAGGGGAACCCAGTCTAAATGAA
GAGCAGGACAAAAGGTTCGTCTGCAAACACTCCATGGTAGACAGAGGATG
GGGAAATGGATGTGGATTATTTGGAAAGGGAGGCATTGTGACCTGTGCTA
TGTTCACATGCAAAAAGAACATGGAAGGAAAAATCGTGCAACCAGAAAAT
TTGGAATACACCATCGTGATAACACCTCACTCAGGAGAAGAGCACGCTGT
AGGTAATGACACAGGAAAACATGGTAAGGAAATTAAAATAACACCACAGA
GTTCCATCACAGAAGCAGAACTGACAGGCTATGGCACAGTCACGATGGAG
TGCTCTCCGAGAACGGGCCTTGACTTCAATGAGATGGTGCTGCTGCAGAT
GGAAGATAAAGCTTGGCTGGTGCACAGGCAATGGTTCCTAGACCTGCCGT
TACCATGGCTACCCGGAGCGGACACACAAGGATCAAATTGGATACAGAAA
GAGACATTGGTCACTTTCAAAAATCCCCACGCGAAGAAGCAGGATGTCGT
TGTTTTAGGATCTCAAGAAGGAGCCATGCACACGGCACTCACAGGGGCCA
CAGAAATCCAGATGTCATCAGGAAACTTACTATTCACAGGACATCTCAAA
TGCAGGCTGAGAATGGACAAACTACAGCTCAAAGGAATGTCATACTCTAT
GTGTACAGGAAAGTTTAAAATTGTGAAGGAAATAGCAGAAACACAACATG
GAACAATAGTTATCAGAGTACAATATGAAGGAGACGGCTCTCCATGTAAG
ATCCCTTTTGAAATAATGGATTTGGAAAAAAGACATGTCTTAGGTCGCCT
GATTACAGTTAATCCGATCGTAACAGAAAAAGATAGCCCAGTCAACATAG
AAGCAGAACCTCCATTCGGAGACAGCTACATCATTATAGGAGTAGAGCCG
GGACAATTGAAACTCAACTGGTTCAAGAAAGGAAGTTCCATCGGCCAAAT
GTTTGAGACGACAATGAGAGGAGCAAAGAGAATGGCCATTTTAGGTGACA
CAGCCTGGGATTTTGGATCTCTGGGAGGAGTGTTTACATCTATAGGAAAG
GCTCTCCACCAAGTTTTCGGAGCAATCTATGGGGCTGCCTTTAGTGGGGT
TTCATGGACTATGAAAATCCTCATAGGAGTCATCATCACATGGATAGGAA
TGAATTCACGTAGCACCTCACTGTCTGTGTCACTAGTATTGGTGGGAATC
ATAACACTGTACTTGGGAGCTATGGTGCAGGCT

### Corresponding Protein prM to E Sequences of the Four Selected Strains

>LAV2 prME protein sequence

(SEQ ID NO: 8)

fhlttrngephmivsrqekgksllfktevgvnmctlmamdlgelcedtit
ykcpllrqnepedidcwcnststwvtygtcttmgehrrekrsvalvphvg
mgletrtetwmssegawkhvqrietwilrhpgftmmaailaytigtthfq
ralifilltavtpsmtMRCIGMSNRDFVEGVSGGSWVDIVLEHGSCVTTM
AKNKPTLDFELIKTEAKQPATLRKYCIEAKLTNTTTESRCPTQGEPSLNE
EQDKRFVCKHSMVDRGWGNGCGLFGKGGIVTCAMFRCKKNMEGKVVQPEN
LEYTIVITPHSGEEHAVGNDTGKHGKEIKITPQSSITEAELTGYGTVTME
CSPRTGLDFNEMVLLQMENKAWLVHRQWFLDLPLPWLPGADTQGSNWIQK
ETLVTFKNPHAKKQDVVVLGSQEGAMHTALTGATEIQMSSGNLLFTGHLK
CRLRMDKLQLKGMSYSMCTGKFKVVKEIAETQHGTIVIRVQYEGDGSPCK
IPFEIMDLEKRHVLGRLITVNPIVTEKDSPVNIEAEPPFGDSYIIIGVEP
GQLKLNWFKKGSSIGQMFETTMRGAKRMAILGDTAWDFGSLGGVFTSIGK
ALHQVFGAIYGAAFSGVSWTMKILIGVIITWIGMNSRSTSLSVTLVLVGI
VTLYLGVMVQA
>LAV2 E protein sequence

(SEQ ID NO: 13)

MRCIGMSNRDFVEGVSGGSWVDIVLEHGSCVTTMAKNKPTLDFELIKTEA
KQPATLRKYCIEAKLTNTTTESRCPTQGEPSLNEEQDKRFVCKHSMVDRG
WGNGCGLFGKGGIVTCAMFRCKKNMEGKVVQPENLEYTIVITPHSGEEHA
VGNDTGKHGKEIKITPQSSITEAELTGYGTVTMECSPRTGLDFNEMVLLQ
MENKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKVVKEIAETQHGTIVIRVQYEGDGSPCKIPFEIMDLEKRHVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVTLVLVGIVTLYLGVMVQA
>LAV2 M protein sequence

(SEQ ID NO: 19)

svalvphvgmgletrtetwmssegawkhvqrietwilrhpgftmmaaila
ytigtthfqralifilltavtpsmt
>BID/V585 prME protein sequence

(SEQ ID NO: 9)

fhlttrngephmivgrqekgksllfktedgvnmctlmaidlgelcedtit
ykcpllrqnepedidcwcnststwvtygtctttgehrrekrsvalvphvg
mgletrtetwmssegawkhvqrietwilrhpgftimaailaytigtthfq
ralifilltavapsmtMRCIGISNRDFVEGVSGGSWVDIVLEHGSCVTTM
AKNKPTLDFELIKTEAKQPATLRKYCIEAKLTNTTTESRCPTQGEPSLNE
EQDKRFICKHSMVDRGWGNGCGLFGKGGIVTCAMFTCKKNMEGKVVQPEN
LEYTIVITPHSGEEHAVGNDTGKHGKEIKITPQSSITEAELTGYGTVTME
CSPRTGLDFNEMVLLQMEDKAWLVHRQWFLDLPLPWLPGADTQGSNWIQK
ETLVTFKNPHAKKQDVVVLGSQEGAMHTALTGATEIQMSSGNLLFTGHLK
CRLRMDKLQLKGMSYSMCTGKFKIVKEIAETQHGTIVIRVQYEGDGSPCK
IPFEIMDLEKRHVLGRLITVNPIVTEKDSPVNIEAEPPFGDSYIIIGVEP
GQLKLNWFKKGSSIGQMFETTMRGAKRMAILGDTAWDFGSLGGVFTSIGK
ALHQVFGAIYGAAFSGVSWTMKILIGVIITWIGMNSRSTSLSVSLVLVGV
VTLYLGVMVQA
>BID/V585 E protein sequence

(SEQ ID NO: 14)

MRCIGISNRDFVEGVSGGSWVDIVLEHGSCVTTMAKNKPTLDFELIKTEA
KQPATLRKYCIEAKLTNTTTESRCPTQGEPSLNEEQDKRFICKHSMVDRG
WGNGCGLFGKGGIVTCAMFTCKKNMEGKVVQPENLEYTIVITPHSGEEHA
VGNDTGKHGKEIKITPQSSITEAELTGYGTVTMECSPRTGLDFNEMVLLQ
MEDKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKIVKEIAETQHGTIVIRVQYEGDGSPCKIPFEIMDLEKRHVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGVVTLYLGVMVQA
>BID/V585 M protein sequence

(SEQ ID NO: 20)

svalvphvgmgletrtetwmssegawkhvqrietwilrhpgfti
maailaytigtthfqralifilltavapsmt
>PR/DB023 prME protein sequence

(SEQ ID NO: 10)

fhlttrngephmivgrqekgksllfktedgvnmctlmaidlgelcedtit
ykcpllrqnepedidcwcnststwvtygtctttgehrrekrsvalvphvg
mgletrtetwmssegawkhvqrietwilrhpgftimaailaytigtthfq
ralifilltavapsmtMRCIGISNRDFVEGVSGGSWVDIVLEHGSCVTTM
AKNKPTLDFELIKTEAKQPATLRKYCIEAKLTNTTTESRCPTQGEPSLNE
EQDKRFICKHSMVDRGWGNGCGLFGKGGIVTCAMFTCKKNMEGKVVLPEN
LEYTIVITPHSGEEHAVGNDTGKHGKEIKITPQSSITEAELTGYGTVTME
CSPRTGLDFNEMVLLQMEDKAWLVHRQWFLDLPLPWLPGADTQGSNWIQK
ETLVTFKNPHAKKQDVVVLGSQEGAMHTALTGATEIQMSSGNLLFTGHLK
CRLRMDKLQLKGMSYSMCTGKFKIVKEIAETQHGTIVIRVQYEGDGSPCK
IPFEIMDLEKRHVLGRLITVNPIVTEKDSPVNIEAEPPFGDSYIIIGVEP
GQLKLNWFKKGSSIGQMFETTMRGAKRMAILGDTAWDFGSLGGVFTSIGK
ALHQVFGAIYGAAFSGVSWTMKILIGVIITWIGMNSRSTSLSVSLVLVGV
VTLYLGVMVQA
>PR/DB023 E protein sequence

(SEQ ID NO: 15)

MRCIGISNRDFVEGVSGGSWVDIVLEHGSCVTTMAKNKPTLDFELIKTEA
KQPATLRKYCIEAKLTNTTTESRCPTQGEPSLNEEQDKRFICKHSMVDRG
WGNGCGLFGKGGIVTCAMFTCKKNMEGKVVLPENLEYTIVITPHSGEEHA
VGNDTGKHGKEIKITPQSSITEAELTGYGTVTMECSPRTGLDFNEMVLLQ
MEDKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKIVKEIAETQHGTIVIRVQYEGDGSPCKIPFEIMDLEKRHVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGVVTLYLGVMVQA
>PR/DB023 M protein sequence

(SEQ ID NO: 21)

svalvphvgmgletrtetwmssegawkhvqrietwilrhpgftimaaila
ytigtthfqralifilltavapsmt
>MD1280 prME protein sequence

(SEQ ID NO: 11)

fhlttrngephmivgrqekgksllfktedgvnmctlmaidlgelcedtit
ykcpllrqnepedidcwcnststwvtygtctttgehrrekrsvalvphvg
mgletrtetwmssegawkhaqrietwilrhpgftimaailaytvgtthfq
ralifillaavapsmtMRCIGISNRDFVEGVSGGSWVDIVLEHGSCVTTM
AKNKPTLDFELIKTEAKQPATLRKYCIEAKLTNTTTESRCPTQGEPSLNE
EQDKRFVCKHSMVDRGWGNGCGLFGKGGIVTCAMFTCKKNMEGKIVQPEN
LEYTIVITPHSGEEHAVGNDTGKHGKEIKITPQSSITEAELTGYGTVTME
CSPRTGLDFNEMVLLQMEDKAWLVHRQWFLDLPLPWLPGADTQGSNWIQK
ETLVTFKNPHAKKQDVVVLGSQEGAMHTALTGATEIQMSSGNLLFTGHLK
CRLRMDKLQLKGMSYSMCTGKFKIVKEIAETQHGTIVIRVQYEGDGSPCK
IPFEIMDLEKRHVLGRLITVNPIVTEKDSPVNIEAEPPFGDSYIIIGVEP
GQLKLNWFKKGSSIGQMFETTMRGAKRMAILGDTAWDFGSLGGVFTSIGK
ALHQVFGAIYGAAFSGVSWTMKILIGVIITWIGMNSRSTSLSVSLVLVGI
ITLYLGAMVQA
>MD1280 E protein sequence

(SEQ ID NO: 16)

MRCIGISNRDFVEGVSGGSWVDIVLEHGSCVTTMAKNKPTLDFELIKTEA
KQPATLRKYCIEAKLTNTTTESRCPTQGEPSLNEEQDKRFVCKHSMVDRG
WGNGCGLFGKGGIVTCAMFTCKKNMEGKIVQPENLEYTIVITPHSGEEHA
VGNDTGKHGKEIKITPQSSITEAELTGYGTVTMECSPRTGLDFNEMVLLQ
MEDKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKIVKEIAETQHGTIVIRVQYEGDGSPCKIPFEIMDLEKRHVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGIITLYLGAMVQA
>MD1280 M protein sequence

(SEQ ID NO: 22)

svalvphvgmgletrtetwmssegawkhaqrietwilrhpgftimaaila
ytvgtthfqralifillaavapsmt
>Consensus M sequence

(SEQ ID NO: 17)

svalvphvgmgletrtetwmssegawkhvqrietwilrhpgftimaaila
ytigtthfqralifilltavapsmt

### Example 3

### Construction of the cDNA Clones Corresponding to the Optimized Serotype 2 Chimeric Viruses and Production of the Encoded Viruses

Construction of chimeric dengue viruses corresponding to the optimized serotype 2 strains is achieved using the Chimerivax™ technology substantially in accordance with the teaching of Chambers, et al. (1999, J. Virology 73(4):3095-3101). Reference may also be made to international patent applications WO 98/37911, WO 03/101397, WO 07/021672, WO 08/007021, WO 08/047023 and WO 08/065315, which detail the analogous processes used to construct CYD-1, CYD2, CYD-3 and CYD-4. Briefly, however, chimeric dengue viruses corresponding to the optimized serotype 2 strains are constructed as follows (N.B. the optimized chimeric dengue viruses are constructed using the genomic backbone of YF strain YF17D204 (YF-VAX®, Sanofi-Pasteur, Swiftwater, PA, USA).

Construction of Plasmid pSP1101
Construction of the YF-VAX cDNA Clone—pJSY2284.1 (pACYC YF-Vax 5-3)

A full-length infectious cDNA clone of YF-VAX is constructed. The full-length infectious cDNA clone is based on the sequence of YF-VAX. A low copy number plasmid pACYC177 (New England Biolabs, Inc., Ipswich, Mass., USA) is used to assemble the full-length cDNA clone.

A DNA sequence named as SP6 YF-Vax 5-3 is synthesized by GeneArt®. The sequence of SP6 YF-Vax 5-3 is designed in a way to facilitate an easy assembly of a full-length YF-Vax cDNA clone. The sequence is 2897 by long and comprises the Xma I-SP6 promoter, the YF-Vax 5′UTR, the capsid, prM, M, part of E which extends to the Apa I site followed by unique sites Mlu I-Sap I-Ngo MI-Aat II-Cla I for assembly, part of NS5 and further extended to 3′ UTR followed by an Nru I site, which is used for run-off. This synthesized DNA sequence is flanked by EcoR V and Xho I sites. After digestion with EcoR V/Xho I, this DNA fragment is then cloned into the Aat II/Xho I sites of low copy number plasmid pACYC177 to replace the 1615 bp Aat II/Xho I fragment. The resulting plasmid pJSY2284.1 (pACYC YF-Vax 5-3) is confirmed by sequence analysis.

RT-PCR and Cloning of the YF-Vax cDNA Fragments Spanning from the Sites Apa I, Mlu I, Sap I, Ngo MI, Aat II and Cla I and Assembly of a Full-Length Infectious cDNA Clone of YF-vax (pJSY2374.5)

The yellow fever vaccine YF-VAX is grown in Vero cells, and the virus particles are concentrated. The viral RNA of YF-VAX is extracted from the concentrated virus and the cDNA copy is made by reverse transcriptase. Five cDNA fragments as shown herein are PCR amplified, TOPO cloned, sequenced and compared to the sequence of YF-VAX 2003. The PCR errors found in each fragment are corrected by either site-directed mutagenesis or fragment switching. There are too many sequence differences found in Ngo MI-Aat II fragment after TOPO cloning, and therefore, this fragment is synthesized by GeneArt®. After final sequence confirmation, the five DNA fragments; Apa I-Mlu I, Mlu I-Sap I, Sap I-Ngo M1, Ngo MI-Aat II, and Aat II-Cla I are isolated and stepwise cloned into the unique sites Apa I, Mlu I, Sap I, Ngo MI, Aat II and Cla I in the plasmid pJSY2284.1 to obtain plasmid pJSY2374.5, which is confirmed to contain the correct sequence of YF-VAX full-length cDNA.

Construction of cDNA for Optimized Chimeric Dengue Virus Derived from the LAV2 Strain (pSP1101)

The strategy is to replace the prM and E genes of the YF-VAX® vaccine strain in the pJSY2374.5 plasmid containing the YF-VAX genome with those of the LAV2 strain, as done previously to build the CYD-1, CYD-2, CYD-3 and CYD-4 dengue vaccines, using the Chimerivax™ technology. The resulting plasmid is pSP1101.

In pJSY2374, restriction sites used for cloning are Xma I and Mlu I. These sites are located upstream and downstream of a 3000 bp fragment which contains: the SP6 promoter, YF17D 5′UTR, YF17D-capsid, YF17D-prM, YF17D-E and the N terminus of YF17D-NS1. A sequence corresponding to this fragment but instead containing the prM and E genes of LAV2 flanked by Xma I and Mlu I sites is synthesized by GeneArt® and cloned into plasmid pMK-RQ (GeneArt®, Life Technologies Ltd, Paisley, U.K.) to create plasmid pMK-RQ-Seq1. Plasmid pJSY2374.5 and pMK-RQ-Seq1 are digested by Xma I and Mlu 1. The Xma I-Mlu I fragment from pMK-RQ-Seq1 is then inserted into plasmid pJSY2374.5 to form plasmid pSP1101. XL-10 Gold Ultracompetent bacteria (Agilent Technologies, CA, USA) are used for transformation, as they are suitable for large plasmids. In a second step, positive clones are transferred into One Shot® TOP10 E. coli (Life Technologies Ltd, Paisley, U.K.), which allows the amplification of large size plasmids in significant amounts.

Plasmid pSP1101 thus allows the expression of LAV2 strain prM and E proteins with a YF-VAX replication engine. The resulting chimeric virus is designated CYD-LAV. Sequencing analysis shows no mutation as compared to the original sequences.

### Construction of Corresponding Plasmids for Strains BID-V585, PR/DB023 and MD1280

An analogous strategy to that described above is used to build the plasmids corresponding to the serotype 2 strains BID-V585, PR/DB023 and MD1280. These plasmids are designated pSP1102 (BID-V585), pSP1103 (PR/DB023) and pSP1104 (MD1280). The resulting chimeric viruses generated from those plasmids are designated CYD-BID, CYD-PR and CYD-MD. Sequence analysis of the generated plasmids shows no mutations compared to the original sequences.

Generation of Chimeric Viruses from Plasmids pSP1101, pSP1102, pSP1103 and pSP1104

In vitro transcription of RNA and generation of viruses is carried out as previously described (Guirakhoo F et al. J. Virol. 2001; 75:7290-304).

### Example 4

### Evaluation of the Immunogenicity and Viremia of the Optimized Serotype 2 Chimeric Viruses in a Monkey Model

### Evaluation of Immunogenicity and Viremia in Monkeys

### Design of the Study

Four groups each containing four Cynomolgus monkeys are defined. The four groups receive the following formulations (containing 5 log₁₀ CCID₅₀ of each CYD dengue serotype):
1. Control tetravalent formulation, i.e. a formulation comprising CYD-1, CYD-2, CYD-3 and CYD-4.
2. CYD-LAV tetravalent formulation, i.e. a formulation comprising CYD-1, CYD-3, CYD-4 and CYD-LAV.
3. CYD-MD tetravalent formulation, i.e. a formulation comprising CYD-1, CYD-3, CYD-4 and CYD-MD.
4. CYD-PR tetravalent formulation, i.e. a formulation comprising CYD-1, CYD-3, CYD-4 and CYD-PR.

Monkeys receive two doses two months apart, as previously described (Guy B et al., Am J Trop Med Hyg. 2009; 80(2):302-11).

### Results

Immunogenicity (SN₅₀ neutralizing response) and viremia are determined as described in the Materials and Methods section of Guy B., et al., Am. J. Trop. Med. Hyg. 2009; 80(2): 302-11.

TABLE 6
SN₅₀ neutralizing responses in monkeys immunized with optimized
chimeric dengue serotype 2 viruses

| | | PD1 | | | | PD2 | | | |
|---|---|---|---|---|---|---|---|---|---|
| | | DEN1 | DEN2 | DEN3 | DEN4 | DEN1 | DEN2 | DEN3 | DEN4 |
| control CYD TV | responders | 4/4 | 2/4 | 1/4 | 4/4 | 4/4 | 1/4 | 4/4 | 4/4 |
| | GMT | 27 | 5 | 7 | 636 | 71 | 8 | 35 | 425 |
| CYD-LAV TV | responders | 4/4 | 2/4 | 4/4 | 4/4 | 4/4 | 4/4 | 4/4 | 4/4 |
| | GMT | 95 | 63 | 19 | 477 | 189 | 95 | 80 | 477 |
| CYD-MD TV | responders | 4/4 | 4/4 | 3/4 | 4/4 | 4/4 | 4/4 | 4/4 | 4/4 |
| | GMT | 33 | 100 | 8 | 63 | 35 | 63 | 16 | 100 |
| CYD-PR TV | responders | 3/4 | 4/4 | 1/4 | 4/4 | 4/4 | 4/4 | 4/4 | 4/4 |
| | GMT | 27 | 63 | 7 | 168 | 109 | 84 | 38 | 212 |

PD: Post-dose; TV: tetravalent formulation

No serotype 2 viremia is observed, regardless of the serotype 2 chimeric virus administered. In respect of immunogenicity responses against DEN2, the tetravalent formulations comprising CYD-LAV, CYD-MD and CYD-PR demonstrate a higher response (both GMTs and number of responding animals) than the control formulation (see Table 6).

### Example 5

### Assessment of Tetravalent Dengue Vaccine Formulations in Flavivirus-Naïve Adults in Mexico

The objective of the present study was to compare the immunogenicity and viremia of a blended tetravalent dengue vaccine comprising CYD-1 (i.e. the particular Chimerivax dengue serotype 1 (CYD-1) strain generated from the prM and E sequences of DEN1 PU0359 (TYP 1 140)), VDV2, CYD-3 (i.e. the particular Chimerivax dengue serotype 3 (CYD-3) strain generated from the prM and E sequences of DEN3 PaH881/88) and CYD-4 (i.e. the particular Chimerivax dengue serotype 4 (CYD-4) strain generated from the prM and E sequences of DEN4 1228 (TVP 980)) with the immunogenicity and viremia of a tetravalent dengue vaccine comprising CYD-1, CYD-2 (i.e. the particular Chimerivax dengue serotype 2 (CYD-2) strain generated from the prM and E sequences of DEN2 PU0218), CYD-3 and CYD-4. See Example 1 for more detail concerning the particular CYD-1, CYD-2, CYD-3 and CYD-4 used in this study.

The relevant nucleotide and protein sequences of the VDV2 strain are as follows:

>VDV2 nucleotide sequence

(SEQ ID NO: 24)

AGUUGUUAGUCUACGUGGACCGACAAAGACAGAUUCUUUGAGGGAGCUAA
GCUCAAUGUAGUUCUAACAGUUUUUUAAUUAGAGAGCAGAUCUCUGAUGA
AUAACCAACGGAAAAAGGCGAAAAACACGCCUUUCAAUAUGCUGAAACGC
GAGAGAAACCGCGUGUCGACUGUGCAACAGCUGACAAAGAGAUUCUCACU
UGGAAUGCUGCAGGGACGAGGACCAUUAAAACUGUUCAUGGCCCUGGUGG
CGUUCCUUCGUUUCCUAACAAUCCCACCAACAGCAGGGAUAUUGAAGAGA
UGGGGAACAAUUAAAAAAUCAAAAGCUAUUAAUGUUUUGAGAGGGUUCAG
GAAAGAGAUUGGAAGGAUGCUGAACAUCUUGAAUAGGAGACGCAGAUCUG
CAGGCAUGAUCAUUAUGCUGAUUCCAACAGUGAUGGCGUUCCAUUUAACC
ACACGUAACGGAGAACCACACAUGAUCGUCAGCAGACAAGAGAAAGGGAA
AAGUCUUCUGUUUAAAACAGAGGUUGGCGUGAACAUGUGUACCCUCAUGG
CCAUGGACCUUGGUGAAUUGUGUGAAGACACAAUCACGUACAAGUGUCCC
CUUCUCAGGCAGAAUGAGCCAGAAGACAUAGACUGUUGGUGCAACUCUAC
GUCCACGUGGGUAACUUAUGGGACGUGUACCACCAUGGGAGAACAUAGAA
GAGAAAAAAGAUCAGUGGCACUCGUUCCACAUGUGCGAAUGGGACUGGAG
ACACGAACUGAAACAUGGAUGUCAUCAGAAGGGGCCUGGAAACAUGUCCA
GAGAAUUGAAACUUGGAUCUUGAGACAUCCAGGCUUCACCAUGAUGGCAG
CAAUCCUGGCAUACACCAUAGGAACGACACAUUUCCAAAGAGCCCUGAUU
UUCAUCUUACUGACAGCUGUCACUCCUUCAAUGACAAUGCGUUGCAUAGG
AAUGUCAAAUAGAGACUUUGUGGAAGGGGUUUCAGGAGGAAGCUGGGUUG
ACAUAGUCUUAGAACAUGGAAGCUGUGUGACGACGAUGGCAAAAAACAAA
CCAACAUUGGAUUUUGAACUGAUAAAAACAGAAGCCAAACAGCCUGCCAC
CCUAAGGAAGUACUGUAUAGAGGCAAAGCUAACCAACACAACAACAGAAU
CUCGCUGCCCAACACAAGGGGAACCCAGCCUAAAUGAAGAGCAGGACAAA
AGGUUCGUCUGCAAACACUCCAUGGUAGACAGAGGAUGGGGAAAUGGAUG
UGGACUAUUUGGAAAGGGAGGCAUUGUGACCUGUGCUAUGUUCAGAUGCA
AAAAGAACAUGGAAGGAAAAGUUGUGCAACCAGAAAACUUGGAAUACACC
AUUGUGAUAACACCUCACUCAGGGGAAGAGCAUGCAGUCGGAAAUGACAC
AGGAAAACAUGGCAAGGAAAUCAAAAUAACACCACAGAGUUCCAUCACAG
AAGCAGAAUUGACAGGUUAUGGCACUGUCACAAUGGAGUGCUCUCCAAGA
ACGGGCCUCGACUUCAAUGAGAUGGUGUUGCUGCAGAUGGAAAAUAAAGC
UUGGCUGGUGCACAGGCAAUGGUUCCUAGACCUGCCGUUACCAUGGUUGC
CCGGAGCGGACACACAAGAGUCAAAUUGGAUACAGAAGGAGACAUUGGUC
ACUUUCAAAAAUCCCCAUGCGAAGAAACAGGAUGUUGUUGUUUUAGGAUC
CCAAGAAGGGGCCAUGCACACAGCACUUACAGGGGCCACAGAAAUCCAAA
UGUCAUCAGGAAACUUACUCUUCACAGGACAUCUCAAGUGCAGGCUGAGA
AUGGACAAGCUACAGCUCAAAGGAAUGUCAUACUCUAUGUGCACAGGAAA
GUUUAAAGUUGUGAAGGAAAUAGCAGAAACACAACAUGGAACAAUAGUUA
UCAGAGUGCAAUAUGAAGGGGACGGCUCUCCAUGCAAGAUCCCUUUUGAG
AUAAUGGAUUUGGAAAAAAGACAUGUCUUAGGUCGCCUGAUUACAGUCAA
CCCAAUUGUGACAGAAAAAGAUAGCCCAGUCAACAUAGAAGCAGAACCUC
CAUUUGGAGACAGCUACAUCAUCAUAGGAGUAGAGCCGGGACAACUGAAG
CUCAACUGGUUUAAGAAAGGAAGUUCUAUCGGCCAAAUGUUUGAGACAAC
AAUGAGGGGGGCGAAGAGAAUGGCCAUUUUAGGUGACACAGCCUGGGAUU
UUGGAUCCUUGGGAGGAGUGUUUACAUCUAUAGGAAAGGCUCUCCACCAA
GUCUUUGGAGCAAUCUAUGGAGCUGCCUUCAGUGGGGUUUCAUGGACUAU
GAAAAUCCUCAUAGGAGUCAUUAUCACAUGGAUAGGAAUGAAUUCACGCA
GCACCUCACUGUCUGUGACACUAGUAUUGGUGGGAAUUGUGACACUGUAU
UUGGGAGUCAUGGUGCAGGCCGAUAGUGGUUGCGUUGUGAGCUGGAAAAA
CAAAGAACUGAAAUGUGGCAGUGGGAUUUUCAUCACAGACAACGUGCACA
CAUGGACAGAACAAUACAAAUUCCAACCAGAAUCCCCUUCAAAACUAGCU
UCAGCUAUCCAGAAAGCCCAUGAAGAGGACAUUUGUGGAAUCCGCUCAGU
AACAAGACUGGAGAAUCUGAUGUGGAAACAAAUAACACCAGAAUUGAAUC
ACAUUCUAUCAGAAAAUGAGGUGAAGUUAACUAUUAUGACAGGAGACAUC
AAAGGAAUCAUGCAGGCAGGAAAACGAUCUCUGCGGCCUCAGCCCACUGA
GCUGAAGUAUUCAUGGAAAACAUGGGGCAAAGCAAAAAUGCUCUCUACAG
AGUCUCAUAACCAGACCUUUCUCAUUGAUGGCCCCGAAACAGCAGAAUGC
CCCAACACAAAUAGAGCUUGGAAUUCGUUGGAAGUUGAAGACUAUGGCUU
UGGAGUAUUCACCACCAAUAUAUGGCUAAAAUUGAAAGAAAAACAGGAUG
UAUUCUGCGACUCAAAACUCAUGUCAGCGGCCAUAAAAGACAACAGAGCC
GUCCAUGCCGAUAUGGGUUAUUGGAUAGAAAGUGCACUCAAUGACACAUG
GAAGAUAGAGAAAGCCUCUUUCAUUGAAGUUAAAAACUGCCACUGGCCAA
AAUCACACACCCUCUGGAGCAAUGGAGUGCUAGAAAGUGAGAUGAUAAUU
CCAAAGAAUCUCGCUGGACCAGUGUCUCAACACAACUAUAGACCAGGCUA
CCAUACACAAAUAACAGGACCAUGGCAUCUAGGUAAGCUUGAGAUGGACU
UUGAUUUCUGUGAUGGAACAACAGUGGUAGUGACUGAGGACUGCGGAAAU
AGAGGACCCUCUUUGAGAACAACCACUGCCUCUGGAAAACUCAUAACAGA
AUGGUGCUGCCGAUCUUGCACAUUACCACCGCUAAGAUACAGAGGUGAGG
AUGGGUGCUGGUACGGGAUGGAAAUCAGACCAUUGAAGGAGAAAGAAGAG
AAUUUGGUCAACUCCUUGGUCACAGCUGGACAUGGGCAGGUCGACAACUU
UUCACUAGGAGUCUUGGGAAUGGCAUUGUUCCUGGAGGAAAUGCUUAGGA
CCCGAGUAGGAACGAAACAUGCAAUACUACUAGUUGCAGUUUCUUUUGUG
ACAUUGAUCACAGGGAACAUGUCCUUUAGAGACCUGGGAAGAGUGAUGGU
UAUGGUAGGCGCCACUAUGACGGAUGACAUAGGUAUGGGCGUGACUUAUC
UUGCCCUACUAGCAGCCUUCAAAGUCAGACCAACUUUUGCAGCUGGACUA
CUCUUGAGAAAGCUGACCUCCAAGGAAUUGAUGAUGACUACUAUAGGAAU
UGUACUCCUCUCCCAGAGCACCAUACCAGAGACCAUUCUUGAGUUGACUG
AUGCGUUAGCCUUAGGCAUGAUGGUCCUCAAAAUGGUGAGAAAUAUGGAA
AAGUAUCAAUUGGCAGUGACUAUCAUGGCUAUCUUGUGCGUCCCAAACGC
AGUGAUAUUACAAAACGCAUGGAAAGUGAGUUGCACAAUAUUGGCAGUGG
UGUCCGUUUCCCCACUGUUCUUAACAUCCUCACAGCAAAAAACAGAUUGG
AUACCAUUAGCAUUGACGAUCAAAGGUCUCAAUCCAACAGCUAUUUUUCU
AACAACCCUCUCAAGAACCAGCAAGAAAAGGAGCUGGCCAUUAAAUGAGG
CUAUCAUGGCAGUCGGGAUGGUGAGCAUUUUAGCCAGUUCUCUCCUAAAA
AAUGAUAUUCCCAUGACAGGACCAUUAGUGGCUGGAGGGCUCCUCACUGU
GUGCUACGUGCUCACUGGACGAUCGGCCGAUUUGGAACUGGAGAGAGCAG
CCGAUGUCAAAUGGGAAGACCAGGCAGAGAUAUCAGGAAGCAGUCCAAUC
CUGUCAAUAACAAUAUCAGAAGAUGGUAGCAUGUCGAUAAAAAAUGAAGA
GGAAGAACAAACACUGACCAUACUCAUUAGAACAGGAUUGCUGGUGAUCU
CAGGACUUUUUCCUGUAUCAAUACCAAUCACGGCAGCAGCAUGGUACCUG
UGGGAAGUGAAGAAACAACGGGCCGGAGUAUUGUGGGAUGUUCCUUCACC
CCCACCCAUGGGAAAGGCUGAACUGGAAGAUGGAGCCUAUAGAAUUAAGC
AAAAAGGGAUUCUUGGAUAUUCCCAGAUCGGAGCCGGAGUUUACAAAGAA
GGAACAUUCCAUACAAUGUGGCAUGUCACACGUGGCGCUGUUCUAAUGCA
UAAAGGAAAGAGGAUUGAACCAACAUGGGCGGACGUCAAGAAAGACCUAA
UAUCAUAUGGAGGAGGCUGGAAGUUAGAAGGAGAAUGGAAGGAAGGAGAA
GAAGUCCAGGUAUUGGCACUGGAGCCUGGAAAAAAUCCAAGAGCCGUCCA
AACGAAACCUGGUCUUUUCAAAACCAACGCCGGAACAAUAGGUGCUGUAU
CUCUGGACUUUUCUCCUGGAACGUCAGGAUCUCCAAUUAUCGACAAAAAA
GGAAAAGUUGUGGGUCUUUAUGGUAAUGGUGUUGUUACAAGGAGUGGAGC
AUAUGUGAGUGCUAUAGCCCAGACUGAAAAAAGCAUUGAAGACAACCCAG
AGAUCGAAGAUCACAUUUUCCGAAAGAGAAGACUGACCAUCAUGGACCUC
CACCCAGGAGCGGGAAAGACGAAGAGAUACCUUCCGGCCAUAGUCAGAGA
AGCUAUAAAACGGGGUUUGAGAACAUUAAUCUUGGCCCCCACUAGAGUUG
UGGCAGCUGAAAUGGAGGAAGCCCUUAGAGGACUUCCAAUAAGAUACCAG
ACCCCAGCCAUCAGAGCUGAGCACACCGGGCGGGAGAUUGUGGACCUAAU
GUGUCAUGCCACAUUUACCAUGAGGCUGCUAUCACCAGUUAGAGUGCCAA
ACUACAACCUGAUUAUCAUGGACGAAGCCCAUUUCACAGACCCAGCAAGU
AUAGCAGCUAGAGGAUACAUCUCAACUCGAGUGGAGAUGGGUGAGGCAGC
UGGGAUUUUUAUGACAGCCACUCCCCCGGGAAGCAGAGACCCAUUUCCUC
AGAGCAAUGCACCAAUCAUAGAUGAAGAAAGAGAAAUCCCUGAACGCUCG
UGGAAUUCCGGACAUGAAUGGGUCACGGAUUUUAAAGGGAAGACUGUUUG
GUUCGUUCCAAGUAUAAAAGCAGGAAAUGAUAUAGCAGCUUGCCUGAGGA
AAAAUGGAAAGAAAGUGAUACAACUCAGUAGGAAGACCUUUGAUUCUGAG
UAUGUCAAGACUAGAACCAAUGAUUGGGACUUCGUGGUUACAACUGACAU
UUCAGAAAUGGGUGCCAAUUUCAAGGCUGAGAGGGUUAUAGACCCCAGAC
GCUGCAUGAAACCAGUCAUACUAACAGAUGGUGAAGAGCGGGUGAUUCUG
GCAGGACCUAUGCCAGUGACCCACUCUAGUGCAGCACAAAGAAGAGGGAG
AAUAGGAAGAAAUCCAAAAAAUGAGAAUGACCAGUACAUAUACAUGGGGG
AACCUCUGGAAAAUGAUGAAGACUGUGCACACUGGAAAGAAGCUAAAAUG
CUCCUAGAUAACAUCAACACGCCAGAAGGAAUCAUUCCUAGCAUGUUCGA
ACCAGAGCGUGAAAAGGUGGAUGCCAUUGAUGGCGAAUACCGCUUGAGAG
GAGAAGCAAGGAAAACCUUUGUAGACUUAAUGAGAAGAGGAGACCUACCA
GUCUGGUUGGCCUACAGAGUGGCAGCUGAAGGCAUCAACUACGCAGACAG
AAGGUGGUGUUUUGAUGGAGUCAAGAACAACCAAAUCCUAGAAGAAAACG
UGGAAGUUGAAAUCUGGACAAAAGAAGGGGAAAGGAAGAAAUUGAAACCC
AGAUGGUUGGAUGCUAGGAUCUAUUCUGACCCACUGGCGCUAAAAGAAUU
UAAGGAAUUUGCAGCCGGAAGAAAGUCUCUGACCCUGAACCUAAUCACAG
AAAUGGGUAGGCUCCCAACCUUCAUGACUCAGAAGGCAAGAGACGCACUG
GACAACUUAGCAGUGCUGCACACGGCUGAGGCAGGUGGAAGGGCGUACAA
CCAUGCUCUCAGUGAACUGCCGGAGACCCUGGAGACAUUGCUUUUACUGA
CACUUCUGGCUACAGUCACGGGAGGGAUCUUUUUAUUCUUGAUGAGCGCA
AGGGGCAUAGGGAAGAUGACCCUGGGAAUGUGCUGCAUAAUCACGGCUAG
CAUCCUCCUAUGGUACGCACAAAUACAGCCACACUGGAUAGCAGCUUCAA
UAAUACUGGAGUUUUUUCUCAUAGUUUUGCUUAUUCCAGAACCUGAAAAA
CAGAGAACACCCCAAGACAACCAACUGACCUACGUUGUCAUAGCCAUCCU
CACAGUGGUGGCCGCAACCAUGGCAAACGAGAUGGGUUUCCUAGAAAAAA
CGAAGAAAGAUCUCGGAUUGGGAAGCAUUGCAACCCAGCAACCCGAGAGC
AACAUCCUGGACAUAGAUCUACGUCCUGCAUCAGCAUGGACGCUGUAUGC
CGUGGCCACAACAUUUGUUACACCAAUGUUGAGACAUAGCAUUGAAAAUU
CCUCAGUGAAUGUGUCCCUAACAGCUAUAGCCAACCAAGCCACAGUGUUA
AUGGGUCUCGGGAAAGGAUGGCCAUUGUCAAAGAUGGACAUCGGAGUUCC
CCUUCUCGCCAUUGGAUGCUACUCACAAGUCAACCCCAUAACUCUCACAG
CAGCUCUUUUCUUAUUGGUAGCACAUUAUGCCAUCAUAGGGCCAGGACUC
CAAGCAAAAGCAACCAGAGAAGCUCAGAAAAGAGCAGCGGCGGGCAUCAU
GAAAAACCCAACUGUCGAUGGAAUAACAGUGAUUGACCUAGAUCCAAUAC
CUUAUGAUCCAAAGUUUGAAAAGCAGUUGGGACAAGUAAUGCUCCUAGUC
CUCUGCGUGACUCAAGUAUUGAUGAUGAGGACUACAUGGGCUCUGUGUGA
GGCUUUAACCUUAGCUACCGGGCCCAUCUCCACAUUGUGGGAAGGAAAUC
CAGGGAGGUUUUGGAACACUACCAUUGCGGUGUCAAUGGCUAACAUUUUU
AGAGGGAGUUACUUGGCCGGAGCUGGACUUCUCUUUUCUAUUAUGAAGAA
CACAACCAACACAAGAAGGGGAACUGGCAACAUAGGAGAGACGCUUGGAG
AGAAAUGGAAAAGCCGAUUGAACGCAUUGGGAAAAAGUGAAUUCCAGAUC
UACAAGAAAAGUGGAAUCCAGGAAGUGGAUAGAACCUUAGCAAAAGAAGG
CAUUAAAAGAGGAGAAACGGACCAUCACGCUGUGUCGCGAGGCUCAGCAA
AACUGAGAUGGUUCGUUGAGAGAAACAUGGUCACACCAGAAGGGAAAGUA
GUGGACCUCGGUUGUGGCAGAGGAGGCUGGUCAUACUAUUGUGGAGGACU
AAAGAAUGUAAGAGAAGUCAAAGGCCUAACAAAAGGAGGACCAGGACACG
AAGAACCCAUCCCCAUGUCAACAUAUGGGUGGAAUCUAGUGCGUCUUCAA
AGUGGAGUUGACGUUUUCUUCAUCCCGCCAGAAAAGUGUGACACAUUAUU
GUGUGACAUAGGGGAGUCAUCACCAAAUCCCACAGUGGAAGCAGGACGAA
CACUCAGAGUCCUUAACUUAGUAGAAAAUUGGUUGAACAACAACACUCAA
UUUUGCAUAAAGGUUCUCAACCCAUAUAUGCCCUCAGUCAUAGAAAAAAU
GGAAGCACUACAAAGGAAAUAUGGAGGAGCCUUAGUGAGGAAUCCACUCU
CACGAAACUCCACACAUGAGAUGUACUGGGUAUCCAAUGCUUCCGGGAAC
AUAGUGUCAUCAGUGAACAUGAUUUCAAGGAUGUUGAUCAACAGAUUUAC
AAUGAGAUACAAGAAAGCCACUUACGAGCCGGAUGUUGACCUCGGAAGCG
GAACCCGUAACAUCGGGAUUGAAAGUGAGAUACCAAACCUAGAUAUAAUU
GGGAAAAGAAUAGAAAAAAUAAAGCAAGAGCAUGAAACAUCAUGGCACUA
UGACCAAGACCACCCAUACAAAACGUGGGCAUACCAUGGUAGCUAUGAAA
CAAAACAGACUGGAUCAGCAUCAUCCAUGGUCAACGGAGUGGUCAGGCUG
CUGACAAAACCUUGGGACGUUGUCCCCAUGGUGACACAGAUGGCAAUGAC
AGACACGACUCCAUUUGGACAACAGCGCGUUUUUAAAGAGAAAGUGGACA
CGAGAACCCAAGAACCGAAAGAAGGCACGAAGAAACUAAUGAAAAUAACA
GCAGAGUGGCUUUGGAAAGAAUUAGGGAAGAAAAAGACACCCAGGAUGUG
CACCAGAGAAGAAUUCACAAGAAAGGUGAGAAGCAAUGCAGCCUUGGGGG
CCAUAUUCACUGAUGAGAACAAGUGGAAGUCGGCACGUGAGGCUGUUGAA
GAUAGUAGGUUUUGGGAGCUGGUUGACAAGGAAAGGAAUCUCCAUCUUGA
AGGAAAGUGUGAAACAUGUGUGUACAACAUGAUGGGAAAAAGAGAGAAGA
AGCUAGGGGAAUUCGGCAAGGCAAAAGGCAGCAGAGCCAUAUGGUACAUG
UGGCUUGGAGCACGCUUCUUAGAGUUUGAAGCCCUAGGAUUCUUAAAUGA
AGAUCACUGGUUCUCCAGAGAGAACUCCCUGAGUGGAGUGGAAGGAGAAG
GGCUGCACAAGCUAGGUUACAUUCUAAGAGACGUGAGCAAGAAAGAGGGA
GGAGCAAUGUAUGCCGAUGACACCGCAGGAUGGGAUACAAAAAUCACACU
AGAAGACCUAAAAAAUGAAGAGAUGGUAACAAACCACAUGGAAGGAGAAC
ACAAGAAACUAGCCGAGGCCAUUUUCAAACUAACGUACCAAAACAAGGUG
GUGCGUGUGCAAAGACCAACACCAAGAGGCACAGUAAUGGACAUCAUAUC
GAGAAGAGACCAAAGAGGUAGUGGACAAGUUGGCACCUAUGGACUCAAUA
CUUUCACCAAUAUGGAAGCCCAACUAAUCAGACAGAUGGAGGGAGAAGGA
GUCUUUAAAAGCAUUCAGCACCUAACAAUCACAGAAGAAAUCGCUGUGCA
AAACUGGUUAGCAAGAGUGGGGCGCGAAAGGUUAUCAAGAAUGGCCAUCA
GUGGAGAUGAUUGUGUUGUGAAACCUUUAGAUGACAGGUUCGCAAGCGCU
UUAACAGCUCUAAAUGACAUGGGAAAGAUUAGGAAAGACAUACAACAAUG
GGAACCUUCAAGAGGAUGGAAUGAUUGGACACAAGUGCCCUUCUGUUCAC
ACCAUUUCCAUGAGUUAAUCAUGAAAGACGGUCGCGUACUCGUUGUUCCA
UGUAGAAACCAAGAUGAACUGAUUGGCAGAGCCCGAAUCUCCCAAGGAGC
AGGGUGGUCUUUGCGGGAGACGGCCUGUUUGGGGAAGUCUUACGCCCAAA
UGUGGAGCUUGAUGUACUUCCACAGACGCGACCUCAGGCUGGCGGCAAAU
GCUAUUUGCUCGGCAGUACCAUCACAUUGGGUUCCAACAAGUCGAACAAC
CUGGUCCAUACAUGCUAAACAUGAAUGGAUGACAACGGAAGACAUGCUGA
CAGUCUGGAACAGGGUGUGGAUUCAAGAAAACCCAUGGAUGGAAGACAAA
ACUCCAGUGGAAACAUGGGAGGAAAUCCCAUACUUGGGGAAAAGAGAAGA
CCAAUGGUGCGGCUCAUUGAUUGGGUUAACAAGCAGGGCCACCUGGGCAA
AGAACAUCCAAGCAGCAAUAAAUCAAGUUAGAUCCCUUAUAGGCAAUGAA
GAAUACACAGAUUACAUGCCAUCCAUGAAAAGAUUCAGAAGAGAAGAGGA
AGAAGCAGGAGUUCUGUGGUAGAAAGCAAAACUAACAUGAAACAAGGCUA
GAAGUCAGGUCGGAUUAAGCCAUAGUACGGAAAAAACUAUGCUACCUGUG
AGCCCCGUCCAAGGACGUUAAAAGAAGUCAGGCCAUCAUAAAUGCCAUAG
CUUGAGUAAACUAUGCAGCCUGUAGCUCCACCUGAGAAGGUGUAAAAAAU
CCGGGAGGCCACAAACCAUGGAAGCUGUACGCAUGGCGUAGUGGACUAGC
GGUUAGGGGAGACCCCUCCCUUACAAAUCGCAGCAACAAUGGGGGCCCAA
GGCGAGAUGAAGCUGUAGUCUCGCUGGAAGGACUAGAGGUUAGAGGAGAC
CCCCCCGAAACAAAAAACAGCAUAUUGACGCUGGGAAAGACCAGAGAUCC
UGCUGUCUCCUCAGCAUCAUUCCAGGCACAGAACGCCAGAAAAUGGAAUG
GUGCUGUUGAAUCAACAGGUUCU
>VDV2 prME nucleotide sequence

(SEQ ID NO: 25)

UUCCAUUUAACCACACGUAACGGAGAACCACACAUGAUCGUCAGCAGACA
AGAGAAAGGGAAAAGUCUUCUGUUUAAAACAGAGGUUGGCGUGAACAUGU
GUACCCUCAUGGCCAUGGACCUUGGUGAAUUGUGUGAAGACACAAUCACG
UACAAGUGUCCCCUUCUCAGGCAGAAUGAGCCAGAAGACAUAGACUGUUG
GUGCAACUCUACGUCCACGUGGGUAACUUAUGGGACGUGUACCACCAUGG
GAGAACAUAGAAGAGAAAAAAGAUCAGUGGCACUCGUUCCACAUGUGCGA
AUGGGACUGGAGACACGAACUGAAACAUGGAUGUCAUCAGAAGGGGCCUG
GAAACAUGUCCAGAGAAUUGAAACUUGGAUCUUGAGACAUCCAGGCUUCA
CCAUGAUGGCAGCAAUCCUGGCAUACACCAUAGGAACGACACAUUUCCAA
AGAGCCCUGAUUUUCAUCUUACUGACAGCUGUCACUCCUUCAAUGACAAU
GCGUUGCAUAGGAAUGUCAAAUAGAGACUUUGUGGAAGGGGUUUCAGGAG
GAAGCUGGGUUGACAUAGUCUUAGAACAUGGAAGCUGUGUGACGACGAUG
GCAAAAAACAAACCAACAUUGGAUUUUGAACUGAUAAAAACAGAAGCCAA
ACAGCCUGCCACCCUAAGGAAGUACUGUAUAGAGGCAAAGCUAACCAACA
CAACAACAGAAUCUCGCUGCCCAACACAAGGGGAACCCAGCCUAAAUGAA
GAGCAGGACAAAAGGUUCGUCUGCAAACACUCCAUGGUAGACAGAGGAUG
GGGAAAUGGAUGUGGACUAUUUGGAAAGGGAGGCAUUGUGACCUGUGCUA
UGUUCAGAUGCAAAAAGAACAUGGAAGGAAAAGUUGUGCAACCAGAAAAC
UUGGAAUACACCAUUGUGAUAACACCUCACUCAGGGGAAGAGCAUGCAGU
CGGAAAUGACACAGGAAAACAUGGCAAGGAAAUCAAAAUAACACCACAGA
GUUCCAUCACAGAAGCAGAAUUGACAGGUUAUGGCACUGUCACAAUGGAG
UGCUCUCCAAGAACGGGCCUCGACUUCAAUGAGAUGGUGUUGCUGCAGAU
GGAAAAUAAAGCUUGGCUGGUGCACAGGCAAUGGUUCCUAGACCUGCCGU
UACCAUGGUUGCCCGGAGCGGACACACAAGAGUCAAAUUGGAUACAGAAG
GAGACAUUGGUCACUUUCAAAAAUCCCCAUGCGAAGAAACAGGAUGUUGU
UGUUUUAGGAUCCCAAGAAGGGGCCAUGCACACAGCACUUACAGGGGCCA
CAGAAAUCCAAAUGUCAUCAGGAAACUUACUCUUCACAGGACAUCUCAAG
UGCAGGCUGAGAAUGGACAAGCUACAGCUCAAAGGAAUGUCAUACUCUAU
GUGCACAGGAAAGUUUAAAGUUGUGAAGGAAAUAGCAGAAACACAACAUG
GAACAAUAGUUAUCAGAGUGCAAUAUGAAGGGGACGGCUCUCCAUGCAAG
AUCCCUUUUGAGAUAAUGGAUUUGGAAAAAAGACAUGUCUUAGGUCGCCU
GAUUACAGUCAACCCAAUUGUGACAGAAAAAGAUAGCCCAGUCAACAUAG
AAGCAGAACCUCCAUUUGGAGACAGCUACAUCAUCAUAGGAGUAGAGCCG
GGACAACUGAAGCUCAACUGGUUUAAGAAAGGAAGUUCUAUCGGCCAAAU
GUUUGAGACAACAAUGAGGGGGGCGAAGAGAAUGGCCAUUUUAGGUGACA
CAGCCUGGGAUUUUGGAUCCUUGGGAGGAGUGUUUACAUCUAUAGGAAAG
GCUCUCCACCAAGUCUUUGGAGCAAUCUAUGGAGCUGCCUUCAGUGGGGU
UUCAUGGACUAUGAAAAUCCUCAUAGGAGUCAUUAUCACAUGGAUAGGAA
UGAAUUCACGCAGCACCUCACUGUCUGUGACACUAGUAUUGGUGGGAAUU
GUGACACUGUAUUUGGGAGUCAUGGUGCAGGCC
>VDV2 E protein sequence

(SEQ ID NO: 26)

MRCIGMSNRDFVEGVSGGSWVDIVLEHGSCVTTMAKNKPTLDFELIKTEA
KQPATLRKYCIEAKLTNTTTESRCPTQGEPSLNEEQDKRFVCKHSMVDRG
WGNGCGLFGKGGIVTCAMFRCKKNMEGKVVQPENLEYTIVITPHSGEEHA
VGNDTGKHGKEIKITPQSSITEAELTGYGTVTMECSPRTGLDFNEMVLLQ
MENKAWLVHRQWFLDLPLPWLPGADTQESNWIQKETLVTFKNPHAKKQDV
VVLGSQEGAMHTALTGATEIQMSSGNLLFTGHLKCRLRMDKLQLKGMSYS
MCTGKFKVVKEIAETQHGTIVIRVQYEGDGSPCKIPFEIMDLEKRHVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVTLVLVGIVTLYLGVMVQA
>VDV2 M protein sequence

(SEQ ID NO: 27)

SVALVPHVRMGLETRTETWMSSEGAWKHVQRIETWILRHPGFTMMAAILA
YTIGTTHFQRALIFILLTAVTPSMT

### Study Design

In an open, randomised, controlled, phase IIa trial, 150 healthy adults aged 18-45 years were enrolled at two centres in Mexico City, which is a dengue non-endemic area. Main exclusion criteria were: pregnancy or breast-feeding, human immunodeficiency virus, hepatitis B or C seropositivity, immunodeficiency or any other chronic illness that could interfere with the results, previous residence in or travel of >2 weeks to areas with high dengue endemicity, a history of flavivirus infection or previous vaccination against flavivirus disease. Women who were capable of conceiving were required to use an effective method of contraception or abstinence for at least 4 weeks before the first injection until at least four weeks after the last injection.

Participants were randomised into two groups and vaccinations were performed on Day 0 and Day 105 (±15 days). The groups received the following formulations:
Group 1: Blended CYD/VDV2 tetravalent formulation, i.e. a formulation comprising CYD-1, CYD-3, CYD4 and VDV2.
Group 2: Control tetravalent formulation (CYD-TDV), i.e. CYD-1, CYD-2, CYD-3 and CYD-4.

The formulations contained 10⁵ CCID₅₀ of each serotype of the CYD viruses and the formulation administered to Group 1 contained 10⁴ CCID₅₀ of the VDV-2 virus.

### Viremia

To evaluate the safety of the vaccines, the presence of CYD-1-4 or VDV-2 was assessed in serum collected 7, 14 and 21 days after each injection. Analyses were performed by the Global Clinical Immunology laboratory (Sanofi Pasteur, Swiftwater, Pa., USA).

Analyses for CYD-1-4 viremia were performed in two steps, as previously described in Poo et al., Pediatr Infect Dis J (2011) 30: e9. Briefly, a first, non-serotype-specific, reverse transcriptase-polymerase chain reaction (RT-PCR) was used to detect the presence of any of the four CYD viruses. Samples that were positive in this first test were then analysed using four CYD serotype-specific quantitative RT-PCRs. In the non-serotype-specific RT-PCR, RNA was extracted from the serum using a commercial kit and an RT-PCR was carried out with primers from the yellow fever core gene sequence. In the serotype-specific RT-PCRs, RNA was again extracted from the serum using a commercial kit and an RT-PCR was carried out with serotype-specific primers from the envelope non-structural protein 1 junction gene sequence for each serotype. A dengue RT-PCR for serotype 2 was performed in group 1 since the tetravalent blending formulation administered to this group contained the VDV-2 virus.

### Immunogenicity

Antibody levels to each of the four dengue virus serotypes were determined by 50% plaque reduction neutralisation test on serum collected 28 days after each injection as well as on day 365 after the first injection. Briefly, serial 2-fold dilutions of heat-inactivated serum were mixed with a constant challenge dose of each dengue serotype DEN-1, -2, -3, or -4 (expressed as plaque forming unit [PFU]/mL). The mixtures were inoculated into wells of a 24-well plate of confluent VERO cell monolayers. After incubation for several days, dengue virus infection is indicated by formation of plaques. The neutralising antibody titre is calculated as the highest reciprocal dilution (1/dil) of serum at which ≧50% reduction in viral plaque count is observed (PRNT50). The lower limit of quantitation of the dengue PRNT50 is 10; samples with titres ≧10 were considered seropositive.

### Results

Formulations were administered to participants in Groups 1 and 2 on day 0 and day 105 of the study. There were no marked differences between the two groups with regard to the injection site or systemic reactogenicity after either the first or the second vaccination. Viremia was assessed in serum collected 7, 14 and 21 days after each injection (Table 7). The neutralising antibody titres were measured 28 days after each injection and on day 365 after the first injection (Table 8).

TABLE 7
Vaccine virus viremia 7, 14, or 21 days after first and second
injections (n (%) with detectable and quantifiable viremia)

| | First injection | | Second injection | |
|---|---|---|---|---|
| | Group 1 | Group 2 | Group 1 | Group 2 |
| | Blended | Tetravalent | Blended | Tetravalent |
| | CYD/ | CYD- | CYD/ | CYD- |
| | VDV | TDV | VDV | TDV |
| Non-serotype specific | | | | |
| N | 29 | 31 | 28 | 29 |
| Detectable viraemia | 27 (93%) | 25 (81%) | 1 (4%) | 1 (3%) |
| Quantifiable viraemia | 1 (3%) | 2 (6%) | 0 | 0 |
| DENV-1 | | | | |
| Detectable viraemia | 1 (3%) | 4 (13%) | 0 | 0 |
| Quantifiable viraemia | 0 | 2 (7%) | 0 | 0 |
| DENV-2 | | | | |
| Detectable viraemia | 0 | 2 (6%) | 0 | 0 |
| Quantifiable viraemia | 0 | 0 | 0 | 0 |
| DENV-3 | | | | |
| Detectable viraemia | 8 (28%) | 7 (23%) | 1 (4%) | 0 |
| Quantifiable viraemia | 0 | 0 | 0 | 0 |
| DENV-4 | | | | |
| Detectable viraemia | 24 (83%) | 21 (68%) | 0 | 0 |
| Quantifiable viraemia | 0 | 3 (1%) | 0 | 0 |

After the first injection, detectable viremia, as determined by the non-serotype specific RT-PCR test, was observed in a similar proportion of participants in both groups (see Table 7). In the majority of cases, viremia was below the lower limit of quantitation. Analysis with the serotype-specific assays showed that CYD-4 was the most commonly detected serotype, followed by CYD-3. After the second injection of the blended CYD/VDV vaccine in Group 1 or the CYD-TDV vaccine in Group 2, viremia was only detected in one participant per group by the non-serotype-specific assay.

Accordingly, there was no significant difference between the levels of viremia induced by the blended CYD/VDV and CYD-TDV.

TABLE 8
Geometric mean titres (95% confidence interval) of
dengue antibodies 28 days after the first and second
injections and 365 days after the first injection

| | Group 1 | |
|---|---|---|
| | CYD/VDV | Group 2 |
| | blended | CYD-TDV |
| First injection | | |
| Serotype 1 | 15 (9; 28) | 17 (10; 31) |
| Serotype 2 | 17 (8; 33) | 32 (16; 65) |
| Serotype 3 | 64 (31; 133) | 23 (13; 39) |
| Serotype 4 | 552 (299; 1019) | 468 (226; 968) |
| Second injection | | |
| Serotype 1 | 54 (30; 96) | 28 (15; 50) |
| Serotype 2 | 152 (79; 293) | 43 (23; 79) |
| Serotype 3 | 127 (71; 229) | 46 (29; 73) |
| Serotype 4 | 246 (159; 382) | 173 (97; 307) |
| 365 days post-dose 1 | | |
| Serotype 1 | 14 (9; 22) | 18 (10; 30) |
| Serotype 2 | 55 (32; 94) | 16 (9; 29) |
| Serotype 3 | 36 (20; 64) | 11 (7; 16) |
| Serotype 4 | 103 (69; 155) | 72 (44; 117) |

It can be seen from Table 8 that the second injection of the blended CYD/VDV vaccine (Group 1) induced higher GMTs against serotype 2 of dengue virus than the CYD-TDV vaccine (Group 2). An improved response to serotype 2 in the blended CYD/VDV group was also observed 365 days after the first dose.

Furthermore, the second injection of the blended CYD/VDV vaccine (Group 1) resulted in an improved neutralising antibody response against all serotypes of dengue virus when compared with the group receiving the CYD-TDV vaccine (Group 2). Importantly, the blended CYD/VDV formulation group demonstrated a more persistent neutralising antibody response against dengue virus than the CYD-TDV group on day 365 after the first injection.

The example therefore shows that, overall, the blended CYD-1, 3, 4/VDV2 vaccine formulation induces stronger and longer lasting immune responses against the dengue virus serotypes than the CYD-TDV vaccine while showing a similar safety profile, as determined by the levels of viremia.

Sequence Listing

| SEQ ID NO. | Sequence |
|---|---|
| 1 | prM + E CYD23 circulating strain nucleotide sequence |
| 2 | prM + E CYD23 circulating strain protein sequence |
| 3 | prM + E consensus serotype 2 protein sequence |
| 4 | prM + E LAV2 nucleotide sequence |
| 5 | prM + E BID/V585 nucleotide sequence |
| 6 | prM + E PR/DB023 nucleotide sequence |
| 7 | prM + E MD1280 nucleotide sequence |
| 8 | prM + E LAV2 protein sequence |
| 9 | prM + E BID/V585 protein sequence |
| 10 | prM + E PR/DB023 protein sequence |
| 11 | prM + E MD1280 protein sequence |
| 12 | E consensus serotype 2 protein sequence |
| 13 | E LAV2 protein sequence |
| 14 | E BID/V585 protein sequence |
| 15 | E PR/DB023 protein sequence |
| 16 | E MD1280 protein sequence |
| 17 | M consensus serotype 2 protein sequence |
| 18 | E CYD23 circulating strain protein sequence |
| 19 | M LAV2 protein sequence |
| 20 | M BID/V585 protein sequence |
| 21 | M PR/DB023 protein sequence |
| 22 | M MD1280 protein sequence |
| 23 | M CYD23 circulating strain protein sequence |
| 24 | Entire nucleotide sequence of VDV2 (RNA equivalent) |
| 25 | prM + E VDV2 nucleotide sequence (RNA equivalent) |
| 26 | E VDV2 protein sequence |
| 27 | M VDV2 protein sequence |

In the listed nucleotide sequences, where a nucleotide sequence is DNA, the nucleotide T may be replaced with the nucleotide U to give the RNA equivalent of that DNA sequence. Similarly, where a nucleotide sequence is RNA, the nucleotide U may be replaced by the nucleotide T to give the equivalent DNA sequence. The DNA sequences listed above constitute the cDNA sequences of the noted dengue viruses and therefore the equivalent RNA sequences constitute the positive strand RNA of those dengue viruses.

1. A dengue virus serotype 2 vaccine composition comprising:
(i) a dengue antigen selected from the group consisting of:
(a) a live attenuated dengue virus;
(b) an inactivated dengue virus;
(c) a live attenuated or inactivated chimeric dengue virus;
(d) a dengue virus-like particle (VLP); and
(e) a combination of two or more of (a) to (d); or
(iii) a nucleic acid construct or viral vector which is able to express in a human cell a dengue antigen which is a dengue VLP;
wherein said dengue antigen comprises a polypeptide having at least 90% identity to SEQ ID NO: 12.

2. A composition as claimed in claim 1, wherein said polypeptide comprises a valine residue at the position within the polypeptide that corresponds to position 251 of SEQ ID NO: 12.

3. A composition as claimed in claim 1 or claim 2, wherein said polypeptide comprises a methionine residue at the position within the polypeptide that corresponds to position 6 of SEQ ID NO: 12.

4. A composition as claimed in any preceding claim, wherein said polypeptide comprises a valine residue at the position within the polypeptide that corresponds to position 129 of SEQ ID NO: 12.

5. A composition as claimed in any preceding claim, wherein said polypeptide comprises an isoleucine residue at the position within the polypeptide that corresponds to position 141 of SEQ ID NO: 12.

6. A composition as claimed in any preceding claim, wherein said polypeptide comprises an isoleucine residue at the position within the polypeptide that corresponds to position 164 of SEQ ID NO: 12.

7. A composition as claimed in any preceding claim, wherein said polypeptide comprises an aspartate residue at the position within the polypeptide that corresponds to position 203 of SEQ ID NO: 12.

8. A composition as claimed in any preceding claim, wherein said polypeptide comprises a threonine residue at the position within the polypeptide that corresponds to position 226 of SEQ ID NO: 12.

9. A composition as claimed in any preceding claim, wherein said polypeptide comprises a glycine residue at the position within the polypeptide that corresponds to position 228 of SEQ ID NO: 12.

10. A composition as claimed in any preceding claim, wherein said polypeptide comprises an isoleucine residue at the position within the polypeptide that corresponds to position 308 of SEQ ID NO: 12.

11. A composition as claimed in any preceding claim, wherein said polypeptide comprises a threonine residue at the position within the polypeptide that corresponds to position 478 of SEQ ID NO: 12.

12. A composition as claimed in any preceding claim, wherein said polypeptide comprises an isoleucine residue at the position within the polypeptide that corresponds to position 484 of SEQ ID NO: 12.

13. A composition as claimed in any preceding claim, wherein said polypeptide comprises an isoleucine residue at the position within the polypeptide that corresponds to position 485 of SEQ ID NO: 12.

14. A composition as claimed in any preceding claim, wherein said polypeptide comprises an alanine residue at the position within the polypeptide that corresponds to position 491 of SEQ ID NO: 12.

15. A composition as claimed in any preceding claim, wherein said dengue antigen comprises a polypeptide having at least 90% identity to SEQ ID NO: 3.

16. A composition as claimed in any one of claims 1 to 14, wherein said polypeptide has at least 90% identity to SEQ ID NO: 3.

17. A composition as claimed in claim 15 or 16, wherein said polypeptide comprises a glycine residue at the position within the polypeptide that corresponds to position 15 of SEQ ID NO: 3.

18. A composition as claimed in any one of claims 15 to 17, wherein said polypeptide comprises a leucine residue at the position within the polypeptide that corresponds to position 24 of SEQ ID NO: 3.

19. A composition as claimed in any one of claims 15 to 18, wherein said polypeptide comprises an isoleucine residue at the position within the polypeptide that corresponds to position 39 of SEQ ID NO: 3.

20. A composition as claimed in any one of claims 15 to 19, wherein said polypeptide comprises a valine residue at the position within the polypeptide that corresponds to position 120 of SEQ ID NO: 3.

21. A composition as claimed in any one of claims 15 to 20, wherein said polypeptide comprises a threonine residue at the position within the polypeptide that corresponds to position 125 of SEQ ID NO: 3.

22. A composition as claimed in any one of claims 1 to 21, wherein said polypeptide comprises: (i) the sequence as set forth in SEQ ID NO: 13 or a sequence having at least 1 and no more than 5 amino acid substitutions with respect to the sequence as set forth in SEQ ID NO: 13; (ii) the sequence as set forth in SEQ ID NO: 14 or a sequence having at least 1 and no more than 5 amino acid substitutions with respect to the sequence as set forth in SEQ ID NO: 14; (iii) the sequence as set forth in SEQ ID NO: 15 or a sequence having at least 1 and no more than 5 amino acid substitutions with respect to the sequence as set forth in SEQ ID NO: 15; (iv) the sequence as set forth in SEQ ID NO: 16 or a sequence having at least 1 and no more than 5 amino acid substitutions with respect to the sequence as set forth in SEQ ID NO: 16; (v) the sequence as set forth in SEQ ID NO: 18 or a sequence having at least 1 and no more than 5 amino acid substitutions with respect to the sequence as set forth in SEQ ID NO: 18 or (vi) the sequence as set forth in SEQ ID NO: 26 or a sequence having at least 1 and no more than 5 amino acid substitutions with respect to the sequence as set forth in SEQ ID NO: 26.

23. A composition as claimed in claim 1, wherein said dengue antigen comprises a polypeptide comprising a sequence selected from the group consisting of SEQ ID NO: 13; SEQ ID NO: 14, SEQ ID NO: 15, SEQ ID NO: 16, SEQ ID NO: 18 and SEQ ID NO: 26.

24. A composition as claimed in claim 23, wherein said dengue antigen comprises a polypeptide comprising a sequence selected from the group consisting of SEQ ID NO: 13 and SEQ ID NO: 16.

25. A composition as claimed in claim 22, wherein said dengue antigen further comprises a polypeptide comprising: (i) the sequence as set forth in SEQ ID NO: 19 or a sequence having at least 1 and no more than 5 amino acid substitutions with respect to the sequence as set forth in SEQ ID NO: 19; (ii) the sequence as set forth in SEQ ID NO: 20 or a sequence having at least 1 and no more than 5 amino acid substitutions with respect to the sequence as set forth in SEQ ID NO: 20; (iii) the sequence as set forth in SEQ ID NO: 21 or a sequence having at least 1 and no more than 5 amino acid substitutions with respect to the sequence as set forth in SEQ ID NO: 21; (iv) the sequence as set forth in SEQ ID NO: 22 or a sequence having at least 1 and no more than 5 amino acid substitutions with respect to the sequence as set forth in SEQ ID NO: 22; (v) the sequence as set forth in SEQ ID NO: 23 or a sequence having at least 1 and no more than 5 amino acid substitutions with respect to the sequence as set forth in SEQ ID NO: 23; (vi) the sequence as set forth in SEQ ID NO: 27 or a sequence having at least 1 and no more than 5 amino acid substitutions with respect to the sequence as set forth in SEQ ID NO: 27.

26. A composition as claimed in claim 23, wherein said dengue antigen further comprises a polypeptide comprising a sequence selected from the group consisting of SEQ ID NO: 19; SEQ ID NO: 20, SEQ ID NO: 21, SEQ ID NO: 22, SEQ ID NO: 23 and SEQ ID NO: 27.

27. A composition as claimed in claim 22, wherein said dengue antigen comprises:
i) a polypeptide having the sequence as set forth in SEQ ID NO: 13 or a polypeptide having at least 1 and no more than 5 amino acid substitutions with respect to the sequence as set forth in SEQ ID NO: 13; and
a polypeptide having the sequence as set forth in SEQ ID NO: 19 or a polypeptide having at least 1 and no more than 5 amino acid substitutions with respect to the sequence as set forth in SEQ ID NO: 19;
ii) a polypeptide having the sequence as set forth in SEQ ID NO: 14 or a polypeptide having at least 1 and no more than 5 amino acid substitutions with respect to the sequence as set forth in SEQ ID NO: 14; and
a polypeptide having the sequence as set forth in SEQ ID NO: 20 or a polypeptide having at least 1 and no more than 5 amino acid substitutions with respect to the sequence as set forth in SEQ ID NO: 20;
iii) a polypeptide having the sequence as set forth in SEQ ID NO: 15 or a polypeptide having at least 1 and no more than 5 amino acid substitutions with respect to the sequence as set forth in SEQ ID NO: 15; and
a polypeptide having the sequence as set forth in SEQ ID NO: 21 or a polypeptide having at least 1 and no more than 5 amino acid substitutions with respect to the sequence as set forth in SEQ ID NO: 21;
iv) a polypeptide having the sequence as set forth in SEQ ID NO: 16 or a polypeptide having at least 1 and no more than 5 amino acid substitutions with respect to the sequence as set forth in SEQ ID NO: 16; and
a polypeptide having the sequence as set forth in SEQ ID NO: 22 or a polypeptide having at least 1 and no more than 5 amino acid substitutions with respect to the sequence as set forth in SEQ ID NO: 22;
v) a polypeptide having the sequence as set forth in SEQ ID NO: 18 or a polypeptide having at least 1 and no more than 5 amino acid substitutions with respect to the sequence as set forth in SEQ ID NO: 18; and
a polypeptide having the sequence as set forth in SEQ ID NO: 23 or a polypeptide having at least 1 and no more than 5 amino acid substitutions with respect to the sequence as set forth in SEQ ID NO: 23; or
vi) a polypeptide having the sequence as set forth in SEQ ID NO: 26 or a polypeptide having at least 1 and no more than 5 amino acid substitutions with respect to the sequence as set forth in SEQ ID NO: 26; and
a polypeptide having the sequence as set forth in SEQ ID NO: 27 or a polypeptide having at least 1 and no more than 5 amino acid substitutions with respect to the sequence as set forth in SEQ ID NO: 27.

28. A composition as claimed in claim 26, wherein said dengue antigen comprises: i) a polypeptide of SEQ ID NO: 13 and a polypeptide of SEQ ID NO: 19; ii) a polypeptide of SEQ ID NO: 14 and a polypeptide of SEQ ID NO: 20; iii) a polypeptide of SEQ ID NO: 15 and a polypeptide of SEQ ID NO: 21; iv) a polypeptide of SEQ ID NO: 16 and a polypeptide of SEQ ID NO: 22; v) a polypeptide of SEQ ID NO: 18 and a polypeptide of SEQ ID NO: 23 or vi) a polypeptide of SEQ ID NO: 26 and a polypeptide of SEQ ID NO: 27.

29. A composition as claimed in claim 15 or claim 16, wherein said dengue antigen comprises a polypeptide comprising a sequence selected from the group consisting of SEQ ID NO: 8; SEQ ID NO: 9, SEQ ID NO: 10 and SEQ ID NO: 11.

30. A composition as claimed in claim 29, wherein said dengue antigen comprises a polypeptide comprising a sequence selected from the group consisting of SEQ ID NO: 8; and SEQ ID NO: 11.

31. A composition as claimed in any preceding claim, wherein said dengue antigen is selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence encoding a protein comprising a polypeptide as defined in any one of claims 1 to 30.

32. A vaccine composition comprising a dengue antigen of serotype 2 selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence encoding a protein comprising the polypeptide or polypeptides as defined in any one of claims 1 to 30.

33. A vaccine composition comprising a dengue antigen of serotype 2 selected from the group consisting of: (a) a live attenuated dengue virus; (b) an inactivated dengue virus; (c) a live attenuated or inactivated chimeric dengue virus; or (d) a combination of two or more of (a) to (c); wherein said dengue antigen comprises a nucleotide sequence having at least 90% sequence identity to a sequence selected from the group consisting of the RNA equivalent of SEQ ID NO: 1, the RNA equivalent of SEQ ID NO: 4, the RNA equivalent of SEQ ID NO: 5, the RNA equivalent of SEQ ID NO: 6, the RNA equivalent of SEQ ID NO: 7 and SEQ ID NO: 25.

34. A composition as claimed in any preceding claim, wherein said composition comprises a live attenuated chimeric dengue virus.

35. A composition as claimed in claim 34, wherein said composition comprises one or more proteins from a dengue virus and one or more proteins from a different flavivirus.

36. A composition as claimed in claim 35, wherein the different flavivirus is a yellow fever virus.

37. A composition as claimed in claim 36, wherein the yellow fever virus is YF-Vax.

38. A composition as claimed in any preceding claim, wherein the composition further comprises a dengue antigen of serotype 1, a dengue antigen of serotype 3 and a dengue antigen of serotype 4.

39. A composition as claimed in claim 38, wherein said dengue antigens of serotypes 1, 3 and 4 are each independently selected from the group consisting of a live attenuated dengue virus and a live attenuated chimeric dengue virus.

40. A composition as claimed in claim 39, wherein said dengue antigens of serotypes 1, 3 and 4 are each a live attenuated chimeric dengue virus in which the genetic backbone of a recipient flavivirus has been modified by exchanging the sequences encoding the prM and E proteins of the recipient flavivirus with the corresponding sequences of a dengue virus.

41. A composition as claimed in claim 40, wherein the recipient flavivirus is a yellow fever virus.

42. A composition as claimed in any one of claims 38 to 41, wherein said dengue antigens of serotypes 1, 3 and 4 are each a live attenuated chimeric dengue virus and the dengue antigen of serotype 2 is a live attenuated dengue virus of serotype 2 which comprises a nucleic acid sequence having at least 90% sequence identity to SEQ ID NO: 24.

43. A pharmaceutical formulation comprising a composition as claimed in any preceding claim and a pharmaceutically acceptable carrier, diluent or excipient.

44. A composition as claimed in any one of claims 1 to 42 for use in therapy.

45. A composition as claimed in any one of claims 1 to 42 for use in a method of protecting a human subject against dengue disease caused by a dengue virus of serotype 2.

46. A composition as claimed in any one of claims 38 to 42 for use in a method of protecting a human subject against dengue disease caused by a dengue virus of serotype 1, serotype 2, serotype 3 or serotype 4.

47. A method of protecting a human subject against dengue disease caused by a dengue virus of serotype 2, wherein said method comprises administering to said subject an effective amount of a composition according to any one of claims 1 to 42.

48. A method of protecting a human subject against dengue disease caused by a dengue virus of serotype 1, serotype 2, serotype 3 or serotype 4, wherein said method comprises administering to said subject an effective amount of a composition according to any one of claims 38 to 42.

49. A kit comprising a composition according to any one of claims 1 to 42 and instructions for use of said composition in a method of protecting a human subject against dengue disease caused by a dengue virus of serotype 2.

50. A kit comprising a composition according to any one of claims 38 to 42 and instructions for use of said composition in a method of protecting a human subject against dengue disease caused by a dengue virus of serotype 1, serotype 2, serotype 3 or serotype 4.


SEQUENCE LISTING (27 sequences)

SEQ ID NO: 1 - DNA, 1983 bp, Artificial Sequence
  Clinical trial circulating strain
ttccatctaaccacacgcaacggagaaccacacatgatcgtcggtatacaggagaaagggaaaagtcttctgttcaaaacagaggatggtgtgaacatgtgcaccctcatggctatggaccttggtgaattgtgtgaagacacaatcacgtacaagtgtcctcttctcaggcagaatgagccagaagacatagactgttggtgcaactccacgtccacgtgggtaacctatgggacctgtaccactacgggagaacataggagagaaaaaagatcagtggcactcgttccacatgtgggaatgggactggagacgcgaaccgaaacatggatgtcatcagaaggggcttggaaacatgcccagagaattgaaacttggatcctgagacatccaggcttcaccataatggcagcaatcctggcatacaccataggaacgacacatttccagagagtcctgattttcatcctactgacagctgtcgctccttcaatgacaatgcgttgcataggaatatcaaatagagactttgtagaaggggtttcaggaggaagttgggttgacatagtcttagaacatggaagctgtgtgacgacgatggcaaaaaacaaaccaacattggatttcgaactgataaaaacggaagccaaacagcctgccaccctaaggaagtactgcatagaagcaaaactaaccaacacaacaacagaatcccgttgcccaacacaaggggaacccagcctaaaagaagagcaggacaagaggttcgtctgcaaacactccatggtagacagaggatggggaaatggatgtggattatttggaaagggaggcattgtgacctgtgctatgttcacatgcaaaaagaacatggaagggaaaatcgtgcaaccagaaaacttggaatacaccattgtggtaacacctcactcaggggaagagcatgcggtcggaaatgacacaggaaaacacggcaaggaaatcaaagtaacaccacagagttccatcacagaagcagaactgacaggttatggcaccgtcacgatggagtgctccccgagaacaggcctcgacttcaatgagatggtgttgctgcagatggaaaataaagcttggctggtgcataggcaatggtttctagacctgccattaccatggctgcccggagcggataaacaagaatcaaattggatacagaaagaaacattggtcactttcaaaaatccccatgcgaagaaacaggatgttgttgttttaggatcccaagaaggggccatgcatacagcactcacaggagccacagaaatccaaatgtcgtcaggaaacttgctcttcactggacatctcaagtgcaggctgagaatggacaagctacagcttaaaggaatgtcatactctatgtgcacaggaaagtttaaagttgtgaaggaaatagcagaaacacaacatggaacgatagttatcagagtgcaatatgaaggggacggctctccatgtaaaattccttttgagataatggatttggaaaaaagatatgtcttaggccgcctgatcacagtcaacccaattgtaacagaaaaagacagcccagtcaacatagaagcagaacctccattcggagacagttacatcatcataggagtagagccgggacaactgaagctcaactggttcaagaaaggaagttctatcggccaaatgtttgagacaacgatgagaggggcgaagagaatggccattttgggtgacacagcctgggacttcggatccctgggaggagtgtttacatctataggaaaagctctccaccaagtctttggagcgatctatggggctgccttcagtggggtttcatggaccatgaaaatcctcataggagtcattatcacatggataggaatgaactcacgcagcacctcactgtctgtgtcactggtactggtgggaattgtgacactgtatttaggagtcatggtgcaggcc

SEQ ID NO: 2 - protein, 661 aa, Artificial Sequence
  Clinical trial circulating strain
FHLTTRNGEPHMIVGIQEKGKSLLFKTEDGVNMCTLMAMDLGELCEDTITYKCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRVLIFILLTAVAPSMTMRCIGISNRDFVEGVSGGSWVDIVLEHGSCVTTMAKNKPTLDFELIKTEAKQPATLRKYCIEAKLTNTTTESRCPTQGEPSLKEEQDKRFVCKHSMVDRGWGNGCGLFGKGGIVTCAMFTCKKNMEGKIVQPENLEYTIVVTPHSGEEHAVGNDTGKHGKEIKVTPQSSITEAELTGYGTVTMECSPRTGLDFNEMVLLQMENKAWLVHRQWFLDLPLPWLPGADKQESNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRYVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQA

SEQ ID NO: 3 - protein, 661 aa, Artificial Sequence
  prM+E universal serotype 2
FHLTTRNGEPHMIVGRQEKGKSLLFKTEDGVNMCTLMAIDLGELCEDTITYKCPLLRQNEPEDIDCWCNSTSTWVTYGTCTTTGEHRREKRSVALVPHVGMGLETRTETWMSSEGAWKHVQRIETWILRHPGFTIMAAILAYTIGTTHFQRALIFILLTAVAPSMTMRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKXVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKXVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 4 - DNA, 1983 bp, Artificial Sequence
  prM+E LAV2
ttccatttaaccacacgtaacggagaaccacacatgatcgtcagcagacaagagaaagggaaaagtcttctgtttaaaacagaggttggcgtgaacatgtgtaccctcatggccatggaccttggtgaattgtgtgaagacacaatcacgtacaagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgattttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccatttggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggcc

SEQ ID NO: 5 - DNA, 1983 bp, Artificial Sequence
  prM+E BID/V585
ttccatttaaccacacgtaatggagaaccacacatgatcgttggtaggcaagagaaagggaaaagtcttctgtttaaaacagaggatggtgttaacatgtgcaccctcatggccatagaccttggtgaattgtgtgaagatacaatcacgtacaagtgccccctcctcaggcaaaatgaaccagaagacatagattgttggtgcaactctacgtccacatgggtaacttatgggacatgtaccaccacaggagaacacagaagagaaaaaagatcagtggcactcgttccacatgtgggcatgggactggagacacgaactgaaacatggatgtcatcagaaggggcctggaaacatgttcagagaattgaaacctggatcttgagacatccaggctttaccataatggcagcaatcctggcatataccataggaacgacacatttccaaagggctctgatcttcattttactgacagccgttgctccttcaatgacaatgcgttgcataggaatatcaaatagagacttcgtagaaggggtttcaggaggaagttgggttgacatagtcttagaacatggaagttgtgtgacgacgatggcaaaaaataaaccaacattggattttgaactgataaaaacagaagccaaacaacctgccactctaaggaagtactgtatagaagcaaagctgaccaatacaacaacagaatctcgttgcccaacacaaggggaacccagtctaaatgaagagcaggacaaaaggttcatctgcaaacactccatggtagacagaggatggggaaatggatgtggattatttggaaagggaggcattgtgacctgtgctatgttcacatgcaaaaagaacatggaaggaaaagtcgtgcagccagaaaatctggaatacaccatcgtgataacacctcactcaggagaagagcacgctgtaggtaatgacacaggaaagcatggcaaggaaatcaaaataacaccacagagctccatcacagaagcagaactgacaggctatggcactgtcacgatggagtgctctccgagaacgggcctcgacttcaatgagatggtactgctgcagatggaagacaaagcttggctggtgcacaggcaatggttcctagacctgccgttaccatggctacccggagcggacacacaaggatcaaattggatacagaaagagacgttggtcactttcaaaaatccccacgcgaagaaacaggacgtcgttgttttaggatctcaagaaggggccatgcacacggcacttacaggggccacagaaatccagatgtcatcaggaaacttactgttcacaggacatctcaagtgtaggctgagaatggacaaattacagcttaaaggaatgtcatactctatgtgtacaggaaagtttaaaattgtgaaggaaatagcagaaacacaacatggaacaatagttatcagagtacaatatgaaggggacggctctccatgtaagattccttttgagataatggatttggaaaaaagacacgtcctaggtcgcctgattacagtgaacccaatcgtaacagaaaaagatagcccagtcaacatagaagcagaacctccattcggagacagctacatcatcataggagtagagccgggacaattgaaactcaattggttcaagaagggaagttccattggccaaatgtttgagacaacaatgagaggagcgaagagaatggccattttaggtgacacagcctgggattttggatccctgggaggagtgtttacatctataggaaaggctctccaccaagttttcggagcaatctatggggctgcttttagtggggtctcatggactatgaaaatcctcataggagttattatcacatggataggaatgaattcacgtagcacctcactgtctgtgtcactagtattggtgggagtcgtgacactgtacttgggggttatggtgcaggct

SEQ ID NO: 6 - DNA, 1983 bp, Artificial Sequence
  prM+E PR/DB023
ttccatttaaccacacgtaatggagaaccacacatgatcgttggtaggcaagagaaagggaaaagtcttctgttcaaaacagaggatggtgttaacatgtgtaccctcatggccatagaccttggtgaattgtgtgaagatacaatcacgtacaagtgccccctcctcaggcaaaatgaaccagaagacatagattgttggtgcaactctacgtccacatgggtaacttatgggacatgtaccaccacaggagaacacagaagagaaaaaagatcagtggcactcgttccacatgtgggcatgggactggagacacgaactgaaacatggatgtcatcagaaggggcctggaaacatgttcagagaattgaaacctggatattgagacatccaggctttaccataatggcagcaatcctggcatataccataggaacgacacatttccaaagggctctgatcttcattttactgacagccgtcgctccttcaatgacaatgcgttgcataggaatatcaaatagagacttcgtagaaggggtttcaggaggaagttgggttgacatagtcttagaacatggaagttgtgtgacgacgatggcaaaaaataaaccaacattggattttgaactgataaaaacagaagccaaacaacctgccactctaaggaagtactgtatagaagcaaagctgaccaatacaacaacagaatctcgttgcccaacacaaggggaacccagtctaaatgaagagcaggacaaaaggttcatctgcaaacactccatggtagacagaggatggggaaatggatgtggattatttggaaaaggaggcattgtaacctgtgctatgttcacatgcaaaaagaacatggaaggaaaagttgtgctgccagaaaatctggaatacaccatcgtgataacacctcactcaggagaagagcacgctgtaggtaatgacacaggaaaacatggcaaggaaattaaaataacaccacagagttccatcacagaagcagaactgacaggctatggcactgtcacgatggagtgctctccgagaacgggcctcgacttcaatgagatggtgctgctgcagatggaagacaaagcctggctggtgcacaggcaatggttcctagatctgccgttaccatggctacccggagcggacacacaaggatcaaattggatacagaaagagacgttggtcactttcaaaaatccccacgcgaagaaacaggacgtcgttgttttaggatctcaagaaggggccatgcacacggcacttacaggggccacagaaatccagatgtcatcaggaaacttactgttcacaggacatctcaagtgtaggctgagaatggacaaattacagcttaaaggaatgtcatactctatgtgtacaggaaagtttaaaattgtgaaggaaatagcagaaacacaacatggaacaatagttatcagagtacaatatgaaggggacggctctccatgtaagattccttttgagataatggatttagaaaaaagacacgtcctaggtcgcctgattacagtgaacccaatcgtaacagaaaaagatagcccagtcaacatagaagcagaacctccattcggagacagctacatcatcataggagtagagccgggacaattgaaactcaattggttcaagaagggaagttccattggccaaatgtttgagacaacaatgagaggagcgaagagaatggccattttaggtgacacagcctgggattttggatccctgggaggagtgtttacatctataggaaaggctctccaccaagttttcggagcaatctatggggctgcttttagtggggtctcatggactatgaaaatcctcataggagttatcatcacatggataggaatgaattcacgtagcacctcactgtctgtgtcactagtattggtgggagtcgtgacactgtacttgggggttatggtgcaggct

SEQ ID NO: 7 - DNA, 1983 bp, Artificial Sequence
  prM+E MD1280
ttccatttaaccacacgaaatggagaaccacacatgatcgttggcagacaagagaaagggaaaagccttctgtttaaaacagaggatggtgtgaacatgtgtaccctcatggccattgatcttggtgaattgtgtgaagatacaatcacgtacaagtgccccctcctcaggcagaatgaaccagaagatatagattgttggtgcaactccacgtccacatgggtaacttatgggacgtgtaccaccacaggagaacacagaagagaaaaaagatcagtggcactcgttccacatgtgggtatgggactggagacacgaactgaaacatggatgtcgtcagaaggggcctggaaacacgctcagagaattgaaacttggatcttgagacatccaggctttaccataatggcagcaatcctggcatataccgtaggaacgacacatttccaaagggccctgattttcatcttactggcagctgtcgctccttcaatgacaatgcgttgcataggaatatcaaatagagactttgtagaaggggtttcaggaggaagctgggttgacatagtcttagaacatggaagttgtgtgacgacaatggcaaaaaataaaccaacactggattttgaactgataaaaacagaagccaaacaacctgccactctaaggaagtactgtatagaggcaaagctgaccaatacaacaacagaatctcgttgcccaacacaaggggaacccagtctaaatgaagagcaggacaaaaggttcgtctgcaaacactccatggtagacagaggatggggaaatggatgtggattatttggaaagggaggcattgtgacctgtgctatgttcacatgcaaaaagaacatggaaggaaaaatcgtgcaaccagaaaatttggaatacaccatcgtgataacacctcactcaggagaagagcacgctgtaggtaatgacacaggaaaacatggtaaggaaattaaaataacaccacagagttccatcacagaagcagaactgacaggctatggcacagtcacgatggagtgctctccgagaacgggccttgacttcaatgagatggtgctgctgcagatggaagataaagcttggctggtgcacaggcaatggttcctagacctgccgttaccatggctacccggagcggacacacaaggatcaaattggatacagaaagagacattggtcactttcaaaaatccccacgcgaagaagcaggatgtcgttgttttaggatctcaagaaggagccatgcacacggcactcacaggggccacagaaatccagatgtcatcaggaaacttactattcacaggacatctcaaatgcaggctgagaatggacaaactacagctcaaaggaatgtcatactctatgtgtacaggaaagtttaaaattgtgaaggaaatagcagaaacacaacatggaacaatagttatcagagtacaatatgaaggagacggctctccatgtaagatcccttttgaaataatggatttggaaaaaagacatgtcttaggtcgcctgattacagttaatccgatcgtaacagaaaaagatagcccagtcaacatagaagcagaacctccattcggagacagctacatcattataggagtagagccgggacaattgaaactcaactggttcaagaaaggaagttccatcggccaaatgtttgagacgacaatgagaggagcaaagagaatggccattttaggtgacacagcctgggattttggatctctgggaggagtgtttacatctataggaaaggctctccaccaagttttcggagcaatctatggggctgcctttagtggggtttcatggactatgaaaatcctcataggagtcatcatcacatggataggaatgaattcacgtagcacctcactgtctgtgtcactagtattggtgggaatcataacactgtacttgggagctatggtgcaggct

SEQ ID NO: 8 - protein, 661 aa, Artificial Sequence
  prM+E LAV2
FHLTTRNGEPHMIVSRQEKGKSLLFKTEVGVNMCTLMAMDLGELCEDTITYKCPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQA

SEQ ID NO: 9 - protein, 661 aa, Artificial Sequence
  prM+E BID/V585
FHLTTRNGEPHMIVGRQEKGKSLLFKTEDGVNMCTLMAIDLGELCEDTITYKCPLLRQNEPEDIDCWCNSTSTWVTYGTCTTTGEHRREKRSVALVPHVGMGLETRTETWMSSEGAWKHVQRIETWILRHPGFTIMAAILAYTIGTTHFQRALIFILLTAVAPSMTMRCIGISNRDFVEGVSGGSWVDIVLEHGSCVTTMAKNKPTLDFELIKTEAKQPATLRKYCIEAKLTNTTTESRCPTQGEPSLNEEQDKRFICKHSMVDRGWGNGCGLFGKGGIVTCAMFTCKKNMEGKVVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 10 - protein, 661 aa, Artificial Sequence
  prM+E PR/DB023
FHLTTRNGEPHMIVGRQEKGKSLLFKTEDGVNMCTLMAIDLGELCEDTITYKCPLLRQNEPEDIDCWCNSTSTWVTYGTCTTTGEHRREKRSVALVPHVGMGLETRTETWMSSEGAWKHVQRIETWILRHPGFTIMAAILAYTIGTTHFQRALIFILLTAVAPSMTMRCIGISNRDFVEGVSGGSWVDIVLEHGSCVTTMAKNKPTLDFELIKTEAKQPATLRKYCIEAKLTNTTTESRCPTQGEPSLNEEQDKRFICKHSMVDRGWGNGCGLFGKGGIVTCAMFTCKKNMEGKVVLPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 11 - protein, 661 aa, Artificial Sequence
  prM+E MD1280
FHLTTRNGEPHMIVGRQEKGKSLLFKTEDGVNMCTLMAIDLGELCEDTITYKCPLLRQNEPEDIDCWCNSTSTWVTYGTCTTTGEHRREKRSVALVPHVGMGLETRTETWMSSEGAWKHAQRIETWILRHPGFTIMAAILAYTVGTTHFQRALIFILLAAVAPSMTMRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKIVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIITLYLGAMVQA

SEQ ID NO: 12 - protein, 495 aa, Artificial Sequence
  E universal
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKXVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKXVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 13 - protein, 495 aa, Artificial Sequence
  E LAV2
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQA

SEQ ID NO: 14 - protein, 495 aa, Artificial Sequence
  E BID/V585
MRCIGISNRDFVEGVSGGSWVDIVLEHGSCVTTMAKNKPTLDFELIKTEAKQPATLRKYCIEAKLTNTTTESRCPTQGEPSLNEEQDKRFICKHSMVDRGWGNGCGLFGKGGIVTCAMFTCKKNMEGKVVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 15 - protein, 495 aa, Artificial Sequence
  E PR/DB023
MRCIGISNRDFVEGVSGGSWVDIVLEHGSCVTTMAKNKPTLDFELIKTEAKQPATLRKYCIEAKLTNTTTESRCPTQGEPSLNEEQDKRFICKHSMVDRGWGNGCGLFGKGGIVTCAMFTCKKNMEGKVVLPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 16 - protein, 495 aa, Artificial Sequence
  E MD1280
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKIVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIITLYLGAMVQA

SEQ ID NO: 17 - protein, 75 aa, Artificial Sequence
  M consensus
SVALVPHVGMGLETRTETWMSSEGAWKHVQRIETWILRHPGFTIMAAILAYTIGTTHFQRALIFILLTAVAPSMT

SEQ ID NO: 18 - protein, 495 aa, Artificial Sequence
  E circulating strain
MRCIGISNRDFVEGVSGGSWVDIVLEHGSCVTTMAKNKPTLDFELIKTEAKQPATLRKYCIEAKLTNTTTESRCPTQGEPSLKEEQDKRFVCKHSMVDRGWGNGCGLFGKGGIVTCAMFTCKKNMEGKIVQPENLEYTIVVTPHSGEEHAVGNDTGKHGKEIKVTPQSSITEAELTGYGTVTMECSPRTGLDFNEMVLLQMENKAWLVHRQWFLDLPLPWLPGADKQESNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRYVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQA

SEQ ID NO: 19 - protein, 75 aa, Artificial Sequence
  M LAV2
SVALVPHVGMGLETRTETWMSSEGAWKHVQRIETWILRHPGFTMMAAILAYTIGTTHFQRALIFILLTAVTPSMT

SEQ ID NO: 20 - protein, 75 aa, Artificial Sequence; identical to SEQ ID NO: 17
  M BID/V585

SEQ ID NO: 21 - protein, 75 aa, Artificial Sequence; identical to SEQ ID NO: 17
  M PR/DB023

SEQ ID NO: 22 - protein, 75 aa, Artificial Sequence
  M MD1280
SVALVPHVGMGLETRTETWMSSEGAWKHAQRIETWILRHPGFTIMAAILAYTVGTTHFQRALIFILLAAVAPSMT

SEQ ID NO: 23 - protein, 75 aa, Artificial Sequence
  M clinical trial circulating strain
SVALVPHVGMGLETRTETWMSSEGAWKHAQRIETWILRHPGFTIMAAILAYTIGTTHFQRVLIFILLTAVAPSMT

SEQ ID NO: 24 - RNA, 10723 nt, Artificial Sequence
  VDV2
aguuguuagucuacguggaccgacaaagacagauucuuugagggagcuaagcucaauguaguucuaacaguuuuuuaauuagagagcagaucucugaugaauaaccaacggaaaaaggcgaaaaacacgccuuucaauaugcugaaacgcgagagaaaccgcgugucgacugugcaacagcugacaaagagauucucacuuggaaugcugcagggacgaggaccauuaaaacuguucauggcccugguggcguuccuucguuuccuaacaaucccaccaacagcagggauauugaagagauggggaacaauuaaaaaaucaaaagcuauuaauguuuugagaggguucaggaaagagauuggaaggaugcugaacaucuugaauaggagacgcagaucugcaggcaugaucauuaugcugauuccaacagugauggcguuccauuuaaccacacguaacggagaaccacacaugaucgucagcagacaagagaaagggaaaagucuucuguuuaaaacagagguuggcgugaacauguguacccucauggccauggaccuuggugaauugugugaagacacaaucacguacaaguguccccuucucaggcagaaugagccagaagacauagacuguuggugcaacucuacguccacguggguaacuuaugggacguguaccaccaugggagaacauagaagagaaaaaagaucaguggcacucguuccacaugugcgaaugggacuggagacacgaacugaaacauggaugucaucagaaggggccuggaaacauguccagagaauugaaacuuggaucuugagacauccaggcuucaccaugauggcagcaauccuggcauacaccauaggaacgacacauuuccaaagagcccugauuuucaucuuacugacagcugucacuccuucaaugacaaugcguugcauaggaaugucaaauagagacuuuguggaagggguuucaggaggaagcuggguugacauagucuuagaacauggaagcugugugacgacgauggcaaaaaacaaaccaacauuggauuuugaacugauaaaaacagaagccaaacagccugccacccuaaggaaguacuguauagaggcaaagcuaaccaacacaacaacagaaucucgcugcccaacacaaggggaacccagccuaaaugaagagcaggacaaaagguucgucugcaaacacuccaugguagacagaggauggggaaauggauguggacuauuuggaaagggaggcauugugaccugugcuauguucagaugcaaaaagaacauggaaggaaaaguugugcaaccagaaaacuuggaauacaccauugugauaacaccucacucaggggaagagcaugcagucggaaaugacacaggaaaacauggcaaggaaaucaaaauaacaccacagaguuccaucacagaagcagaauugacagguuauggcacugucacaauggagugcucuccaagaacgggccucgacuucaaugagaugguguugcugcagauggaaaauaaagcuuggcuggugcacaggcaaugguuccuagaccugccguuaccaugguugcccggagcggacacacaagagucaaauuggauacagaaggagacauuggucacuuucaaaaauccccaugcgaagaaacaggauguuguuguuuuaggaucccaagaaggggccaugcacacagcacuuacaggggccacagaaauccaaaugucaucaggaaacuuacucuucacaggacaucucaagugcaggcugagaauggacaagcuacagcucaaaggaaugucauacucuaugugcacaggaaaguuuaaaguugugaaggaaauagcagaaacacaacauggaacaauaguuaucagagugcaauaugaaggggacggcucuccaugcaagaucccuuuugagauaauggauuuggaaaaaagacaugucuuaggucgccugauuacagucaacccaauugugacagaaaaagauagcccagucaacauagaagcagaaccuccauuuggagacagcuacaucaucauaggaguagagccgggacaacugaagcucaacugguuuaagaaaggaaguucuaucggccaaauguuugagacaacaaugaggggggcgaagagaauggccauuuuaggugacacagccugggauuuuggauccuugggaggaguguuuacaucuauaggaaaggcucuccaccaagucuuuggagcaaucuauggagcugccuucagugggguuucauggacuaugaaaauccucauaggagucauuaucacauggauaggaaugaauucacgcagcaccucacugucugugacacuaguauuggugggaauugugacacuguauuugggagucauggugcaggccgauagugguugcguugugagcuggaaaaacaaagaacugaaauguggcagugggauuuucaucacagacaacgugcacacauggacagaacaauacaaauuccaaccagaauccccuucaaaacuagcuucagcuauccagaaagcccaugaagaggacauuuguggaauccgcucaguaacaagacuggagaaucugauguggaaacaaauaacaccagaauugaaucacauucuaucagaaaaugaggugaaguuaacuauuaugacaggagacaucaaaggaaucaugcaggcaggaaaacgaucucugcggccucagcccacugagcugaaguauucauggaaaacauggggcaaagcaaaaaugcucucuacagagucucauaaccagaccuuucucauugauggccccgaaacagcagaaugccccaacacaaauagagcuuggaauucguuggaaguugaagacuauggcuuuggaguauucaccaccaauauauggcuaaaauugaaagaaaaacaggauguauucugcgacucaaaacucaugucagcggccauaaaagacaacagagccguccaugccgauauggguuauuggauagaaagugcacucaaugacacauggaagauagagaaagccucuuucauugaaguuaaaaacugccacuggccaaaaucacacacccucuggagcaauggagugcuagaaagugagaugauaauuccaaagaaucucgcuggaccagugucucaacacaacuauagaccaggcuaccauacacaaauaacaggaccauggcaucuagguaagcuugagauggacuuugauuucugugauggaacaacagugguagugacugaggacugcggaaauagaggacccucuuugagaacaaccacugccucuggaaaacucauaacagaauggugcugccgaucuugcacauuaccaccgcuaagauacagaggugaggaugggugcugguacgggauggaaaucagaccauugaaggagaaagaagagaauuuggucaacuccuuggucacagcuggacaugggcaggucgacaacuuuucacuaggagucuugggaauggcauuguuccuggaggaaaugcuuaggacccgaguaggaacgaaacaugcaauacuacuaguugcaguuucuuuugugacauugaucacagggaacauguccuuuagagaccugggaagagugaugguuaugguaggcgccacuaugacggaugacauagguaugggcgugacuuaucuugcccuacuagcagccuucaaagucagaccaacuuuugcagcuggacuacucuugagaaagcugaccuccaaggaauugaugaugacuacuauaggaauuguacuccucucccagagcaccauaccagagaccauucuugaguugacugaugcguuagccuuaggcaugaugguccucaaaauggugagaaauauggaaaaguaucaauuggcagugacuaucauggcuaucuugugcgucccaaacgcagugauauuacaaaacgcauggaaagugaguugcacaauauuggcagugguguccguuuccccacuguucuuaacauccucacagcaaaaaacagauuggauaccauuagcauugacgaucaaaggucucaauccaacagcuauuuuucuaacaacccucucaagaaccagcaagaaaaggagcuggccauuaaaugaggcuaucauggcagucgggauggugagcauuuuagccaguucucuccuaaaaaaugauauucccaugacaggaccauuaguggcuggagggcuccucacugugugcuacgugcucacuggacgaucggccgauuuggaacuggagagagcagccgaugucaaaugggaagaccaggcagagauaucaggaagcaguccaauccugucaauaacaauaucagaagaugguagcaugucgauaaaaaaugaagaggaagaacaaacacugaccauacucauuagaacaggauugcuggugaucucaggacuuuuuccuguaucaauaccaaucacggcagcagcaugguaccugugggaagugaagaaacaacgggccggaguauugugggauguuccuucacccccacccaugggaaaggcugaacuggaagauggagccuauagaauuaagcaaaaagggauucuuggauauucccagaucggagccggaguuuacaaagaaggaacauuccauacaauguggcaugucacacguggcgcuguucuaaugcauaaaggaaagaggauugaaccaacaugggcggacgucaagaaagaccuaauaucauauggaggaggcuggaaguuagaaggagaauggaaggaaggagaagaaguccagguauuggcacuggagccuggaaaaaauccaagagccguccaaacgaaaccuggucuuuucaaaaccaacgccggaacaauaggugcuguaucucuggacuuuucuccuggaacgucaggaucuccaauuaucgacaaaaaaggaaaaguugugggucuuuaugguaaugguguuguuacaaggaguggagcauaugugagugcuauagcccagacugaaaaaagcauugaagacaacccagagaucgaagaucacauuuuccgaaagagaagacugaccaucauggaccuccacccaggagcgggaaagacgaagagauaccuuccggccauagucagagaagcuauaaaacgggguuugagaacauuaaucuuggcccccacuagaguuguggcagcugaaauggaggaagcccuuagaggacuuccaauaagauaccagaccccagccaucagagcugagcacaccgggcgggagauuguggaccuaaugugucaugccacauuuaccaugaggcugcuaucaccaguuagagugccaaacuacaaccugauuaucauggacgaagcccauuucacagacccagcaaguauagcagcuagaggauacaucucaacucgaguggagaugggugaggcagcugggauuuuuaugacagccacucccccgggaagcagagacccauuuccucagagcaaugcaccaaucauagaugaagaaagagaaaucccugaacgcucguggaauuccggacaugaaugggucacggauuuuaaagggaagacuguuugguucguuccaaguauaaaagcaggaaaugauauagcagcuugccugaggaaaaauggaaagaaagugauacaacucaguaggaagaccuuugauucugaguaugucaagacuagaaccaaugauugggacuucgugguuacaacugacauuucagaaaugggugccaauuucaaggcugagaggguuauagaccccagacgcugcaugaaaccagucauacuaacagauggugaagagcgggugauucuggcaggaccuaugccagugacccacucuagugcagcacaaagaagagggagaauaggaagaaauccaaaaaaugagaaugaccaguacauauacaugggggaaccucuggaaaaugaugaagacugugcacacuggaaagaagcuaaaaugcuccuagauaacaucaacacgccagaaggaaucauuccuagcauguucgaaccagagcgugaaaagguggaugccauugauggcgaauaccgcuugagaggagaagcaaggaaaaccuuuguagacuuaaugagaagaggagaccuaccagucugguuggccuacagaguggcagcugaaggcaucaacuacgcagacagaaggugguguuuugauggagucaagaacaaccaaauccuagaagaaaacguggaaguugaaaucuggacaaaagaaggggaaaggaagaaauugaaacccagaugguuggaugcuaggaucuauucugacccacuggcgcuaaaagaauuuaaggaauuugcagccggaagaaagucucugacccugaaccuaaucacagaaauggguaggcucccaaccuucaugacucagaaggcaagagacgcacuggacaacuuagcagugcugcacacggcugaggcagguggaagggcguacaaccaugcucucagugaacugccggagacccuggagacauugcuuuuacugacacuucuggcuacagucacgggagggaucuuuuuauucuugaugagcgcaaggggcauagggaagaugacccugggaaugugcugcauaaucacggcuagcauccuccuaugguacgcacaaauacagccacacuggauagcagcuucaauaauacuggaguuuuuucucauaguuuugcuuauuccagaaccugaaaaacagagaacaccccaagacaaccaacugaccuacguugucauagccauccucacagugguggccgcaaccauggcaaacgagauggguuuccuagaaaaaacgaagaaagaucucggauugggaagcauugcaacccagcaacccgagagcaacauccuggacauagaucuacguccugcaucagcauggacgcuguaugccguggccacaacauuuguuacaccaauguugagacauagcauugaaaauuccucagugaaugugucccuaacagcuauagccaaccaagccacaguguuaaugggucucgggaaaggauggccauugucaaagauggacaucggaguuccccuucucgccauuggaugcuacucacaagucaaccccauaacucucacagcagcucuuuucuuauugguagcacauuaugccaucauagggccaggacuccaagcaaaagcaaccagagaagcucagaaaagagcagcggcgggcaucaugaaaaacccaacugucgauggaauaacagugauugaccuagauccaauaccuuaugauccaaaguuugaaaagcaguugggacaaguaaugcuccuaguccucugcgugacucaaguauugaugaugaggacuacaugggcucugugugaggcuuuaaccuuagcuaccgggcccaucuccacauugugggaaggaaauccagggagguuuuggaacacuaccauugcggugucaauggcuaacauuuuuagagggaguuacuuggccggagcuggacuucucuuuucuauuaugaagaacacaaccaacacaagaaggggaacuggcaacauaggagagacgcuuggagagaaauggaaaagccgauugaacgcauugggaaaaagugaauuccagaucuacaagaaaaguggaauccaggaaguggauagaaccuuagcaaaagaaggcauuaaaagaggagaaacggaccaucacgcugugucgcgaggcucagcaaaacugagaugguucguugagagaaacauggucacaccagaagggaaaguaguggaccucgguuguggcagaggaggcuggucauacuauuguggaggacuaaagaauguaagagaagucaaaggccuaacaaaaggaggaccaggacacgaagaacccauccccaugucaacauauggguggaaucuagugcgucuucaaaguggaguugacguuuucuucaucccgccagaaaagugugacacauuauugugugacauaggggagucaucaccaaaucccacaguggaagcaggacgaacacucagaguccuuaacuuaguagaaaauugguugaacaacaacacucaauuuugcauaaagguucucaacccauauaugcccucagucauagaaaaaauggaagcacuacaaaggaaauauggaggagccuuagugaggaauccacucucacgaaacuccacacaugagauguacuggguauccaaugcuuccgggaacauagugucaucagugaacaugauuucaaggauguugaucaacagauuuacaaugagauacaagaaagccacuuacgagccggauguugaccucggaagcggaacccguaacaucgggauugaaagugagauaccaaaccuagauauaauugggaaaagaauagaaaaaauaaagcaagagcaugaaacaucauggcacuaugaccaagaccacccauacaaaacgugggcauaccaugguagcuaugaaacaaaacagacuggaucagcaucauccauggucaacggaguggucaggcugcugacaaaaccuugggacguuguccccauggugacacagauggcaaugacagacacgacuccauuuggacaacagcgcguuuuuaaagagaaaguggacacgagaacccaagaaccgaaagaaggcacgaagaaacuaaugaaaauaacagcagaguggcuuuggaaagaauuagggaagaaaaagacacccaggaugugcaccagagaagaauucacaagaaaggugagaagcaaugcagccuugggggccauauucacugaugagaacaaguggaagucggcacgugaggcuguugaagauaguagguuuugggagcugguugacaaggaaaggaaucuccaucuugaaggaaagugugaaacauguguguacaacaugaugggaaaaagagagaagaagcuaggggaauucggcaaggcaaaaggcagcagagccauaugguacauguggcuuggagcacgcuucuuagaguuugaagcccuaggauucuuaaaugaagaucacugguucuccagagagaacucccugaguggaguggaaggagaagggcugcacaagcuagguuacauucuaagagacgugagcaagaaagagggaggagcaauguaugccgaugacaccgcaggaugggauacaaaaaucacacuagaagaccuaaaaaaugaagagaugguaacaaaccacauggaaggagaacacaagaaacuagccgaggccauuuucaaacuaacguaccaaaacaagguggugcgugugcaaagaccaacaccaagaggcacaguaauggacaucauaucgagaagagaccaaagagguaguggacaaguuggcaccuauggacucaauacuuucaccaauauggaagcccaacuaaucagacagauggagggagaaggagucuuuaaaagcauucagcaccuaacaaucacagaagaaaucgcugugcaaaacugguuagcaagaguggggcgcgaaagguuaucaagaauggccaucaguggagaugauuguguugugaaaccuuuagaugacagguucgcaagcgcuuuaacagcucuaaaugacaugggaaagauuaggaaagacauacaacaaugggaaccuucaagaggauggaaugauuggacacaagugcccuucuguucacaccauuuccaugaguuaaucaugaaagacggucgcguacucguuguuccauguagaaaccaagaugaacugauuggcagagcccgaaucucccaaggagcaggguggucuuugcgggagacggccuguuuggggaagucuuacgcccaaauguggagcuugauguacuuccacagacgcgaccucaggcuggcggcaaaugcuauuugcucggcaguaccaucacauuggguuccaacaagucgaacaaccugguccauacaugcuaaacaugaauggaugacaacggaagacaugcugacagucuggaacaggguguggauucaagaaaacccauggauggaagacaaaacuccaguggaaacaugggaggaaaucccauacuuggggaaaagagaagaccaauggugcggcucauugauuggguuaacaagcagggccaccugggcaaagaacauccaagcagcaauaaaucaaguuagaucccuuauaggcaaugaagaauacacagauuacaugccauccaugaaaagauucagaagagaagaggaagaagcaggaguucugugguagaaagcaaaacuaacaugaaacaaggcuagaagucaggucggauuaagccauaguacggaaaaaacuaugcuaccugugagccccguccaaggacguuaaaagaagucaggccaucauaaaugccauagcuugaguaaacuaugcagccuguagcuccaccugagaagguguaaaaaauccgggaggccacaaaccauggaagcuguacgcauggcguaguggacuagcgguuaggggagaccccucccuuacaaaucgcagcaacaaugggggcccaaggcgagaugaagcuguagucucgcuggaaggacuagagguuagaggagacccccccgaaacaaaaaacagcauauugacgcugggaaagaccagagauccugcugucuccucagcaucauuccaggcacagaacgccagaaaauggaauggugcuguugaaucaacagguucu

SEQ ID NO: 25 - RNA, 1983 nt, Artificial Sequence
  prM+E VDV2
uuccauuuaaccacacguaacggagaaccacacaugaucgucagcagacaagagaaagggaaaagucuucuguuuaaaacagagguuggcgugaacauguguacccucauggccauggaccuuggugaauugugugaagacacaaucacguacaaguguccccuucucaggcagaaugagccagaagacauagacuguuggugcaacucuacguccacguggguaacuuaugggacguguaccaccaugggagaacauagaagagaaaaaagaucaguggcacucguuccacaugugcgaaugggacuggagacacgaacugaaacauggaugucaucagaaggggccuggaaacauguccagagaauugaaacuuggaucuugagacauccaggcuucaccaugauggcagcaauccuggcauacaccauaggaacgacacauuuccaaagagcccugauuuucaucuuacugacagcugucacuccuucaaugacaaugcguugcauaggaaugucaaauagagacuuuguggaagggguuucaggaggaagcuggguugacauagucuuagaacauggaagcugugugacgacgauggcaaaaaacaaaccaacauuggauuuugaacugauaaaaacagaagccaaacagccugccacccuaaggaaguacuguauagaggcaaagcuaaccaacacaacaacagaaucucgcugcccaacacaaggggaacccagccuaaaugaagagcaggacaaaagguucgucugcaaacacuccaugguagacagaggauggggaaauggauguggacuauuuggaaagggaggcauugugaccugugcuauguucagaugcaaaaagaacauggaaggaaaaguugugcaaccagaaaacuuggaauacaccauugugauaacaccucacucaggggaagagcaugcagucggaaaugacacaggaaaacauggcaaggaaaucaaaauaacaccacagaguuccaucacagaagcagaauugacagguuauggcacugucacaauggagugcucuccaagaacgggccucgacuucaaugagaugguguugcugcagauggaaaauaaagcuuggcuggugcacaggcaaugguuccuagaccugccguuaccaugguugcccggagcggacacacaagagucaaauuggauacagaaggagacauuggucacuuucaaaaauccccaugcgaagaaacaggauguuguuguuuuaggaucccaagaaggggccaugcacacagcacuuacaggggccacagaaauccaaaugucaucaggaaacuuacucuucacaggacaucucaagugcaggcugagaauggacaagcuacagcucaaaggaaugucauacucuaugugcacaggaaaguuuaaaguugugaaggaaauagcagaaacacaacauggaacaauaguuaucagagugcaauaugaaggggacggcucuccaugcaagaucccuuuugagauaauggauuuggaaaaaagacaugucuuaggucgccugauuacagucaacccaauugugacagaaaaagauagcccagucaacauagaagcagaaccuccauuuggagacagcuacaucaucauaggaguagagccgggacaacugaagcucaacugguuuaagaaaggaaguucuaucggccaaauguuugagacaacaaugaggggggcgaagagaauggccauuuuaggugacacagccugggauuuuggauccuugggaggaguguuuacaucuauaggaaaggcucuccaccaagucuuuggagcaaucuauggagcugccuucagugggguuucauggacuaugaaaauccucauaggagucauuaucacauggauaggaaugaauucacgcagcaccucacugucugugacacuaguauuggugggaauugugacacuguauuugggagucauggugcaggcc

SEQ ID NO: 26 - protein, 495 aa, Artificial Sequence
  E VDV2
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQESNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQA

SEQ ID NO: 27 - protein, 75 aa, Artificial Sequence
  M VDV2
SVALVPHVRMGLETRTETWMSSEGAWKHVQRIETWILRHPGFTMMAAILAYTIGTTHFQRALIFILLTAVTPSMT